(19) United States
(12) Patent Application Publication
(10) Pub. No.: US 2024/0294673 A1
(43) Pub. Date: Sep. 5, 2024

(54) RECOMBINANT PROTEINACEOUS BINDING MOLECULES

(71) Applicant: JULIUS-MAXIMILIANS-UNIVERSITAET WUERZBURG, Wuerzburg (DE)

(72) Inventors: Gernot STUHLER, Wuerzburg (DE); Tim SCHNYDER, Wuerzburg (DE); Boris NOWOTNY, Wuerzburg (DE); Annika BREMER, Wuerzburg (DE)

(21) Appl. No.: 18/564,921

(22) PCT Filed: May 27, 2022
(86) PCT No.: PCT/EP2022/064401

§ 371 Date: Nov. 28, 2023

(30) Foreign Application Priority Data
May 28, 2021 (EP) 21176655.5

(51) Int. Cl. C07K 16/46; A61K 39/00; C07K 16/28; C07K 16/30; C07K 16/32

(52) U.S. Cl. CPC C07K 16/468; C07K 16/2863; C07K 16/30; C07K 16/32; A61K 2039/505; C07K 2317/31; C07K 2317/526; C07K 2317/64; C07K 2317/73; C07K 2317/74; C07K 2317/94

(57) ABSTRACT

The present invention relates to a recombinant proteinaceous binding molecule wherein an Fc fragment is used as fusion partner and combined it with the hemibody technology. Furthermore, the invention relates to a heterodimeric recombinant proteinaceous binding molecule, as well as a method for producing the same, its use and a nucleic acid molecule encoding the recombinant proteinaceous binding molecule. The invention in particular provides a proteinaceous binding molecule that is capable of mediating target cell restricted activation of immune cells.

### CROSS-REFERENCE TO RELATED APPLICATIONS

The present application claims the benefit of priority of European Patent Application No. 21176655.5 filed 28 May 2021, the content of which is hereby incorporated by reference it its entirety for all purposes.

### TECHNICAL FIELD OF THE INVENTION

The present invention relates to a recombinant proteinaceous binding molecule, as well as a method for producing the same, its use and a nucleic acid molecule encoding the conditionally active, recombinant proteinaceous binding molecule. The invention in particular provides a recombinant proteinaceous binding molecule that is capable of mediating target cell restricted activation of immune cells.

### BACKGROUND

The first established cancer immunotherapy comprised monoclonal antibodies (mAbs). These mAbs are structurally identical to natural antibodies and can combat cancer cells with different mechanisms, including direct and immune mediated tumor cell killing. One direct effect can be caused via antibodies that bind epidermal growth factor receptors. These receptors are thereby blocked, which results in an inhibition of their activity, loss of stimulatory signals and subsequent cell cycle arrest followed by apoptosis (Redman J M, Hill E M, AIDeghaither D, Weiner L M. Mechanisms of action of therapeutic antibodies for cancer. Molecular Immunology 2015, 67:2845). Immune mediated tumor cell killing occurs based on the Fc fragment. One typical mechanism is the antibody-dependent cell-mediated cytotoxicity (ADCC). When the Ab's variable region binds its specific antigen on the surface of a target cell, leukocytes can bind the Fc region via Fey receptors. The binding of NK cells causes a recruitment of adapter proteins and activation of the NK cell. This leads to the release of lytic factors like granzyme and perforin with subsequent target cell destruction. ADCP (antibody-dependent cell phagocytosis) occurs if Fc fragments of mAbs are recognized by Fey receptors of macrophages. Tumor cells tagged with mAbs get phagocytosed and thus eliminated. A third immune mediated tumor cell killing is the complement mediated cytotoxicity (CDC). The complement component C1 recognizes the Fc Fragment and activates the classical complement pathway. This results in the formation of a membrane attack complex (MAC) causing pores within the cell membrane with subsequent cell lysis. Two mAbs have been approved by the Food and Drug Administration (FDA) for relapsed/refractory multiple myeloma in 2015. Daratumumab is a human IgG1 mAb against CD38 and induces ADCP, ADCC and CDC. (Overdijk M B, Verploegen S, Bogels M, van Egmond M, van Lammerts Bueren J J, Mutis T, et al. Antibody-mediated phagocytosis contributes to the anti-tumor activity of the therapeutic antibody daratumumab in lymphoma and multiple myeloma. mAbs 2015; 7:311-21; Weers M de, Tai Y-T, van der Veer M S, Bakker J M, Vink T, Jacobs D C H, et al. Daratumumab, a novel therapeutic human CD38 monoclonal antibody, induces killing of multiple myeloma and other hematological tumors. Journal of immunology (Baltimore, Md.: 1950) 2011; 186:1840-48). Elotuzumab is a humanized mAb targeting SlamF7, approved as combination therapy with lenalidomide and dexamethasone (Lonial S, Dimopoulos M, Palumbo A, White D, Grosicki S, Spicka 1, et al. Elotuzumab Therapy for Relapsed or Refractory Multiple Myeloma. The New England journal of medicine 2015; 373:621-31) and causes NK mediated ADCC (Collins S M, Bakan C E, Swartzel G D, Hofmeister C C, Efebera Y A, Kwon H, et al. Elotuzumab directly enhances NK cell cytotoxicity against myeloma via CSI ligation: evidence for augmented NK cell function complementing ADCC. Cancer immunology, immunotherapy: CM 2013; 62:1841-49). Another innovative development in the field of targeted immunotherapy can be seen in chimeric antigen receptor (CAR) T cells. These CAR T cells are genetically modified and express synthetic receptors with specificities against tumor antigens. Therefore, T cells are isolated from the patient's blood, transduced with a CAR protein, expanded and tested in vitro and transferred back to the patient. A CAR protein comprises a single-chain variable fragment (scFv) with binding capacity for one specific tumor associated antigen, linked via a transmembrane peptide to intracellular co-stimulatory domains such as CD28, 0X40 and CD137. These peptides are subsequently joined to the signaling domains of the chain that activates the CAR T cell, if it binds its epitope on a tumor cell. Subsequent release of granzymes and perforns leads to tumor cell lysis (June C H, O'Connor R S, Kawalekar O U, Ghassemi S, Milone M C. CAR T cell immunotherapy for human cancer. Science (New York, N.Y.) 2018; 359:1361-65). These synthetic receptor molecules enable a MHC independent T cell activation unlike the common reaction via the TCR complex. (Gideon Gross, Tova Waks, and Zelig Eshhar. Expression of immunoglobulin-T-cell receptor chimeric molecules as functional receptors with antibody-type specificity. Proc. Natl. Acad. Sci. USA 1989:10024-28; Kuwana Yea. Expression of chimeric receptor composed of immunoglobulin-derived V regions and T-cell receptor-derived C regions. Biochemical and biophysical research communications 1987:960-68). This represents an important benefit in tumor cell recognition since the loss of MHC-associated antigen presentation is a major immune escape strategy by malignant cells (Garrido F, Aptsiauri N, Doorduijn E M, Garcia Lora A M, van Hall T. The urgent need to recover MHC class I in cancers for effective immunotherapy. Current opinion in immunology 2016; 39:44-51). To date the most promising results could be generated with CAR T cells with specificity against CD19 that is also present on multiple myeloma cells (Garfall A L, Maus M V, Hwang W-T, Lacey S F, Mahnke Y D, Melenhorst J J, et al. Chimeric Antigen Receptor T Cells against CD19 for Multiple Myeloma. The New England journal of medicine 2015:373:1040-47) In 2017 the FDA approved an antiCD19 CAR T cell therapy for the treatment of relapsed or refractory acute lymphoblastic leukemia and large B-cell lymphomas. (Eric Tran, Dan L. Lonngo, and Walter J. Urban. A Milestone for CAR T Cells. The New England journal of medicine 2017; 377:2593-96). CD38 and SlamF7 are also possible targets for CAR-T cells against multiple myeloma (Mihara K, Yanagihara K, Takigahira M, Kitanaka A, Imai C, Bhattacharyya J, et al. Synergistic and persistent effect of T-cell immunotherapy with anti-CD19 or anti-CD38 chimeric receptor in conjunction with rituximab on B-cell non-Hodgkin lymphoma. British journal of haematology 2010; 151:37-46; Gogishvili T, Danhof S, Prommersberger S, Rydzek J, Schreder M, Brede C, et al. SLAMF7-CAR T cells eliminate myeloma and confer selective fratricide of SLAMF7+ normal lymphocytes. Blood 2017; 130:2838-47).

To make it possible to address different targets at the same time strategies to design bispecific antibodies have been developed. The first bispecific antibodies were structurally identical to immunoglobulin G (IgG) molecules but had two different Fab fragments with dissimilar antigen specificities. Indeed, they showed insufficient effectivity in clinical trials resulting in no further development (Baeuerle P A, Reinhardt C. Bispecific T-cell engaging antibodies for cancer therapy. Cancer research 2009; 69:4941-44). Instead, small bispecific antibodies lacking the Fc part were designed by Mack and colleagues. Two scFv with different binding specificity were combined in tandem, using a small peptide linker (Matthias Mack, Gert Riethmuller, Peter Kufer. A small bispecific antibody construct expressed as a functional single-chain molecule with high tumor cell cytotoxicity. Proc. Natl. Acad. Sci. USA 1995:7021-25). ScFv are antibodies reduced to their minimal binding domains consisting of the heavy (VH) and light chain (VL) of the variable fragment of an IgG, joined with a serine-glycine linker sequence. The first small bispecific antibody directed against the 17-1A antigen and the CD3 antigen on T lymphocytes was designed to be expressed in CHO cells as one functional single chain molecule. An efficient purification from the culture medium was ensured by adding a C-terminal histidine tail and a N-terminal flag epitope was inserted for easy detection. The resulting recombinant protein showed high cytotoxicity for 17-1 A positive tumor cells at nanomolar concentrations (Matthias Mack, Gert Riethmuller, Peter Kufer. A small bispecific antibody construct expressed as a functional single-chain molecule with high tumor cell cytotoxicity. Proc. Natl. Acad. Sci. USA 1995:7021-25). With use of this technology “bispecific T cell engagers” (BiTEs) were designed. In these molecules one scFv specifically binds the TCR complex mostly by targeting the CD3 subunit which allows a T cell activation. The second binding domain is designed to be specific for a selected tumor antigen, which should be minimally expressed on healthy tissue to avoid on-target off-tumor effects. The capability of BiTEs to recruit cytotoxic T cells towards the tumor cells and induce specific tumor killing is a huge advantage in comparison to mAbs (Huehls A M, Coupet T A, Sentman C L. Bispecific T-cell engagers for cancer immunotherapy. Immunology and cell biology 2015; 93:290-96). BiTes showed high efficiency in clinical trials. The first clinically approved BiTe is Blinatumomab, targeting CD3 and CD19, for treatment of relapsed/refractory B-cell derived acute lymphoblastic leukemia (Wu J, Fu J, Zhang M, Liu D. Blinatumomab: a bispecific T cell engager (BiTE) antibody against CD19/CD3 for refractory acute lymphoid leukemia. Journal of hematology & oncology 2015; 8:104). Reducing the antibodies to a smaller size (50 kDa instead of 150 kDa) leads to better tumor penetration but also has some disadvantages. The persistence of full antibody molecules in the blood is maintained by an Fc-Receptor (FcR) mediated recycling mechanism. The lack of the Fc part causes relatively short serum half-lives with an average of 1.25 h in humans (Huehls A M, Coupet T A, Sentman C L. Bispecific T-cell engagers for cancer immunotherapy. Immunology and cell biology 2015; 93:290-96).

BiTEs and CAR-T cells are effective novel therapeutic options for specific cancer types but there are also some limitations. However, as mentioned before, the target antigen has to be highly expressed on tumor cells but rarely present in healthy tissue to prevent severe side effects.

Furthermore, although by reducing the therapeutic antibodies to small fragments, a better tumor penetration is achieved, this also leads to disadvantages like the fast elimination from the body. In sum, there is still a need to increase the specificity of T-cell engaging antibodies, and improving their half-life while maintaining high tumor penetration. The technical problem underlying the present application is thus to comply with these needs. The technical problem is solved by the embodiments as defined in the claims, described in the description and illustrated in the examples and figures that follow.

### SUMMARY OF THE INVENTION

In a first aspect, the present invention relates to a recombinant proteinaceous binding molecule comprising:
a) a binding moiety, capable of binding an antigen, comprising a first binding site for a first antigen,
b) a variable domain of either an antibody light chain or an antibody heavy chain of a second binding site for a second antigen,
c) an Fc fragment comprising a first and a second heavy chain wherein the first and the second heavy chain each comprise one immunoglobulin CH2 domain and one immunoglobulin CH3 domain, wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface (formed by the association of the respective CH2 domains with each other and by the association of the respective CH3 domains with each other, meaning this interface comprises an original interface between the CH3 domains),
wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and wherein the variable domain of either the antibody light chain or the antibody heavy chain and the first binding moiety are linked via the Fc fragment.

In a second aspect the present invention relates to a heterodimeric recombinant proteinaceous binding molecule comprising a heterodimer of recombinant proteinaceous molecules (monomers). Accordingly, the first monomer consists of: a) a binding moiety having a first binding site for a first antigen; b) a variable domain of either an antibody light chain or an antibody heavy chain of a second binding site for a second antigen; and c) an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable. In said recombinant proteinaceous binding molecule the variable domain of either the antibody light chain or the antibody heavy chain and the first binding moiety are linked via the Fc fragment. Furthermore, the second monomer of said heterodimeric recombinant proteinaceous binding molecule consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, which interface comprises an original interface between the CH3 domains, wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and wherein the variable domain of either the antibody light chain or the antibody heavy chain and the first binding moiety are linked via the Fc fragment. In this heteorimeric recombinant preotinaceous binding molecule, the first antigen of the first monomer and the first antigen of the second monomer are two antigens of the same identity. Furthermore, the variable domain of an antibody light chain of the second binding site of the first monomer and the variable domain of an antibody heavy chain of the second binding site of the second monomer associate, thereby forming the second binding site and dimerizing the heterodimer.

In a third aspect, the present invention relates to a heterodimeric recombinant proteinaceous binding molecule comprising a heterodimer of recombinant proteinaceous molecules (monomers), wherein the first antigen of the first monomer and the first antigen of the second monomer are two antigens of different identity. Accordingly, the first monomer consists of: a) a binding moiety having a first binding site for a first antigen; b) a variable domain of either an antibody light chain or an antibody heavy chain of a second binding site for a second antigen; and c) an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, which interface comprises an original interface between the CH3 domains wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable. In said recombinant proteinaceous binding molecule the variable domain of either the antibody light chain or the antibody heavy chain and the first binding moiety are linked via the Fc fragment. Furthermore, the second monomer of said heterodimeric recombinant proteinaceous binding molecule consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain each meet each other at an interface, which interface comprises an original interface between the CH3 domains, wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and wherein the variable domain of either the antibody light chain or the antibody heavy chain and the first binding moiety are linked via the Fc fragment. In this heteorimeric recombinant preotinaceous binding molecule, the first antigen of the first monomer and the first antigen of the second monomer are two antigens of the different identity. Furthermore, the variable domain of an antibody light chain of the second binding site of the first monomer and the variable domain of an antibody heavy chain of the second binding site of the second monomer associate, thereby forming the second binding site and dimerizing the heterodimer.

In a fourth aspect the present invention provides a pharmaceutical composition comprising a recombinant proteinaceous binding molecule of the present invention.

In a fifth aspect the present invention provides a recombinant proteinaceous binding molecule for use in the treatment or diagnosis of a disease.

In a sixth aspect the present invention relates to a nucleic acid molecule encoding a recombinant proteinaceous binding molecule of the present invention.

In a seventh aspect the invention provides a nucleic acid molecule of the present invention comprised in a vector.

In an eight aspect the invention provides a host cell comprising a nucleic acid molecule or the vector of the present invention.

In a ninth aspect the present invention provides the use of the recombinant proteinaceous binding molecule of the present invention for the treatment of a disease, wherein the recombinant proteinaceous binding molecule of the present invention forms a heterodimer only in vivo on a target cell, thereby reducing “off target activation”.

In a tenth aspect the invention provides a method of producing a recombinant proteinaceous binding molecule of the present invention, comprising expressing a nucleic acid encoding the recombinant proteinaceous binding molecule under conditions allowing expression of the nucleic acid.

These aspects of the invention will be more fully understood in view of the following description, drawings and non-limiting examples.

### BRIEF DESCRIPTION OF THE DRAWINGS

The Figures show.

FIG. 1: Knob-into-hole hemibody (KiHss) format (technology). FIG. 1A shows the general construct design of a disulfide stabilized Knob into Hole hemibody of the invention (KiHss): “A” is the binding moiety, “B” is the variable domain of an antibody light chain or heavy chain, straight lines represent the linker or “hinge” region, disulfide bridges are depicted by dotted lines with the letter “S” and a purification or detection tag (e.g. Strep®-II tag, polyhistidine-tag, myc tag, flag tag, HA tag) may be fused at either the C- or N-terminus of either the Fc knob or the Fc hole chain. FIG. 1 B shows a mechanistic model of KiHss hemibody triggered tumor destruction: Fc Fragments were fused to hemibody constructs to create the KiHss hemibodies of the invention. T cell recruitment occurred after simultaneous binding of two hemibodies to their specific targets on the same cell with subsequent T cell activation and target cell elimination. FIG. 1C shows the full length sequence of human IgG constant heavy chain (Uniprot ID P01857) with numbering starting with amino-acid No. 1 and with numbering starting at amino-acid 118 (according to EU numbering) Legend: Bold=CH1; Underlined=hinge region; Light grey=CH2; Dark grey=CH3. For EU numbering see also www.imgt.org/AMGTScientificChart/Numbering/Hu_IGHGnber.html. In bold are indicated the CH1 regions, underlined are the hinge regions, in light grey are the CH2 regions and in dark grey the CH3 regions. FIG. 1D shows the knob and hole mutations used for the KiHss hemibodies of the invention. FIG. 1E shows the effector silencing mutations used for the KiHss hemibodies of the present invention.

FIG. 2: Electrophoretic separation of SEC purified hemibody constructs. The separation was performed under non-reducing conditions (shown in FIG. 2A) and reducing conditions (shown in FIG. 2B) using a 4-20% SDS-polyacrylamide gradient gel followed by Coomassie blue G250 staining. For analysis 0.5 pg of each hemibody was loaded onto the gel. Lane 1, VL antiEpcam; Lane 2, VH antiEpcam, Lane 3 VL antiEGFR, Lane 4, VH antiEGFR; Lane 5, VL antiHer2.

FIG. 3: Hemibody mediated target-cell lysis. The lysis assay was done with serial diluted (dilution factor 3) hemibody pair (shown in FIG. 3A) VH antiEpcam+VL antiHer2 and (shown in FIG. 3B) VH antiEpcam+VL antiEGFR starting at a concentration of 5 nM for each hemibody with purified CD8 positive T-cells as effector cells and Chinese hamster ovary cells (CHO), co-expressing target antigens as indicated. nM, Nanomolar

FIG. 4: Test for activity of single hemibody constructs. To test if single hemibodies can affect cell viability, effector and target cells were co-incubated in the presence of 5 nM single hemibody construct. As negative control, cells were incubated without construct. Purified CD8 positive T-cells were used as effector cells and Chinese hamster ovary cells (CHO) as target cells, co-expressing target-antigens as indicated.

FIG. 5: Hemibody mediated T-cell activation. The activation assay was performed with serial diluted (dilution factor 3) hemibody pair (shown in FIG. 5A) VH antiEpcam+VL antiHer2 and (shown in FIG. 5B) VH antiEpcam+VL antiEGFR starting at a concentration of 5 nM for each hemibody with Jurkat Lucia NFAT cells as effector cells and target-antigen co-expressing Chinese hamster ovary cells (CHO) as target cells. nM, Nanomolar; RLI, relative luminescence intensity.

FIG. 6: Test for activity of single hemibody constructs. To test if single hemibodies can activate T-cells, effector and target cells were co-incubated in the presence of 5 nM single hemibody construct. As negative control cells were incubated without construct. Jurkat Lucia NFAT cells were used as effector cells and target-antigen co expressing Chinese hamster ovary cells (CHO) as target cells. RLI, relative luminescence intensity

FIG. 7: Hemibody mediated T-cell activation in the absence and presence of a target-antigen competitor. For competition, Trastuzumab was used in the full antibody and in the single chain variable fragment (scFv) format. The competition assay was performed with serial diluted (dilution factor 2) hemibody construct pair VH antiEGFR+VL antiHer2 (shown in FIG. 7A) and VH antiEpcam+VL antiHer2 (shown in FIG. 7B) starting at 5 nM for each hemibody and at constant competitor concentration of 100 nM. Effector and target cells were incubated with competitors for 2 h before addition of hemibody constructs. As effector cells Jurkat Lucia NFAT cells and as target cells firefly luciferase expressing T47D (A) and A549 (B) cells were used. RLI, relative luminescence intensity; nM, Nanomolar.

FIG. 8: Hemibody mediated T-cell activation in the absence and presence of a target-antigen competitor. For competition Trastuzumab was used in the full antibody and in the single chain variable fragment (scFv) format. The competition assay was performed using hemibody pair VH antiEpcam+VL antiHer2 at a concentration of 2 nM for each hemibody construct and a competitor concentration of 50 nM. Effector and target cells were incubated with competitor for 2 h before addition of hemibodies. As effector cells Jurkat Lucia NFAT cells and as target cells hamster ovary cells (CHO) stably co-expressing huEpcam and huHer2 were used. RLI, relative luminescence intensity.

FIG. 9: Hemibody mediated T-cell activation. The activation assay was performed with serial diluted (dilution factor 2) hemibody constructs VL antiEGFR and VL anti Her2 starting at 2 nM and at constant VH antiEpcam construct concentration of 1 nM. As effector cells Jurkat Lucia NFAT cells and as target cells firefly luciferase expressing A549 (shown in FIG. 9A) and MDA-MB-468 (shown in FIG. 9B) cells were used. RLI, relative luminescence intensity; nM, Nanomolar.

FIG. 10: Hemibody mediated T-cell activation. The activation assay was performed with serial diluted (dilution factor 2) hemibody constructs VH antiEpcam and VL anti Her2 starting at 2 nM. As effector cells Jurkat Lucia NFAT cells and as target cells firefly luciferase expressing A549 and MDA-MB-468 cells were used. RLI, relative luminescence intensity; nM, Nanomolar.

FIG. 11: Antigen density status of used tumor cell lines A549, MDA-MB-468 and T47D. Cells were stained with target specific APC conjugated antibodies and subjected

to flow cytometric analysis. Solid, target antigen; Line, isotype control; Bold, median target antigen signal intensity; underlined, median of isotype control signal intensity.

FIG. 12: Hemibody mediated T-cell activation. The activation assay was performed with serial diluted (dilution factor 2) hemibody constructs VH antiEpcam and VL anti EGFR starting at 2 nM. Jurkat Lucia NFAT cells were used as effector and firefly luciferase expressing MDA-MB-231 and MDA-MB-453 as target cells. RLI, relative luminescence intensity; nM, Nanomolar.

FIG. 13: Antigen density status of used tumor cell lines MDA-MB-453 and MDA-MB-231. Cells were stained with target specific APC conjugated antibodies and subjected to flow cytometric analysis. Solid, target antigen; Line, isotype control; Bold, median target antigen signal intensity; underlined, median of isotype control signal intensity.

FIG. 14: Hemibody mediated T-cell activation. The activation assay was performed with serial diluted (dilution factor 2) hemibody constructs VH antiEpcam and VL anti Her2 starting at 2 nM. As effector cells Jurkat Lucia NFAT cells and as target cells double target-antigen positive Chinese hamster ovary cells (CHO) were used as indicated. RLI, relative luminescence intensity; nM, Nanomolar.

FIG. 15: Hemibody mediated T-cell activation. The activation assay was performed with the hemibody pairs VH antiEpcam+VL anti Her2, VH antiEpcam+VL anti EGFR and VH antiEGFR+VL antiHer2 at a concentration of 2 nM for each construct on Chinese hamster ovary cells (CHO) cells dual positive for huEpcam and huEGFR. As effector cells Jurkat Lucia NFAT cells were used. RLI, relative luminescence intensity.

FIG. 16: Antigen density status of engineered double target-antigen positive CHO cells. Cells were stained with target specific FITC conjugated antibodies and subjected to flow cytometric analysis. FIG. 16A: Solid black, antihuEpcam FITC; Solid grey, antihuHer2 FITC; Solid line, negative control (only cells, no antibody); Dashed line, antihuEGFR FITC; Bold, Median huHer2 signal intensity; in brackets, Median huEpcam signal intensity; Underlined, Median negative control. FIG. 16B: Solid black, antihuEpcam FITC; Solid grey, antihuEGFR FITC; Solid line, negative control (only cells, no antibody); Dashed line, antihuHer2 FITC; Bold, Median huEGFR signal intensity; in brackets, Median huEpcam signal intensity; Underlined, Median negative control. FIG. 16C: Solid black, antihuEGFR FITC; Solid grey, antihuHer2 FITC; Solid line, negative control (only cells, no antibody); Dashed line, antihuEpcam FITC; Bold, Median huHer2 signal intensity; in brackets, Median huEGFR signal intensity; Underlined, Median negative control.

FIG. 17: Target-antigen binding of purified hemibodies. Quantification of Hemibody binding onto single-target-antigen positive engineered CHO cells was done by detecting the HIS-tag using a HIS-tag specific APC antibody conjugate and flow cytometry.

Solid black, VL antiEpcam; Solid grey, VH antiEpcam; Solid line, negative control (only antiHIS APC antibody conjugate).

FIG. 18: Target-antigen binding of purified hemibodies. Quantification of Hemibody binding onto single-target-antigen positive engineered CHO cells was done by detecting the HIS-tag using a HIS-tag specific APC antibody conjugate and flow cytometry. Solid black, VL antiEGFR, Solid grey, VH antiEGFR; Solid line, negative control (only antiHIS APC antibody conjugate).

FIG. 19: Target-antigen binding of purified hemibodies. Quantification of Hemibody binding onto single-target-antigen positive engineered CHO cells was done by detecting the HIS-tag using a HIS-tag specific APC antibody conjugate and flow cytometry. Solid black, VL antiHer2; Solid line, negative control (only antiHIS APC antibody conjugate).

FIG. 20: Antigen density status of engineered single-target-antigen positive CHO cells. Cells were stained with target antigen specific APC antibody conjugates and subjected to flow cytometric analysis. FIG. 20A: Solid black, antihuEpcam APC; Solid line, negative control (only cells, no antibody); Dashed line, antihuEGFR APC; Dotted line, antihuHer2 APC; Bold, Median huEpcam signal intensity; Underlined, Median negative control. FIG. 20B: Solid black, antihuHer2 APC; Solid line, negative control (only cells, no antibody); Dashed line, antihuEGFR APC; Dotted line, antiEpcam APC; Bold, Median huHer2 signal intensity; Underlined, Median negative control. FIG. 20C: Solid black, antihuEGFR APC; Solid line, negative control (only cells, no antibody); Dashed line, antihuHer2 APC; Dotted line, antiEpcam APC; Bold, Median huEGFR signal intensity; Underlined, Median negative control.

FIG. 21: Size exclusion chromatography profile of hemibody construct VL antiHer2. Quality evaluation of the SEC purified hemibody constructs. Purified hemibodies were analyzed for aggregation and purity using the AKTApure system and Superdex 200 Increase 5/150 GL size exclusion column. As molecular weight standard protein beta-amylase (200 kDa) and carbonic anhydrase (29 kDa) was used. mAU, milli-arbitrary units; mL, milli-litre.

FIG. 22: Size exclusion chromatography profile of hemibody constructs VH antiEpcam and VL antiEpcam. Quality evaluation of the SEC purified hemibody constructs. Purified hemibodies were analyzed for aggregation and purity using the AKTApure system and Superdex 200 5/150 Increase size exclusion column. As molecular weight standard protein beta-amylase (200 kDa) and carbonic anhydrase (29 kDa) was used. mAU, milli-arbitrary units; mL, milli-litre.

FIG. 23: Size exclusion chromatography profile of hemibody constructs VH antiEGFR and VL antiEGFR. Quality evaluation of the SEC purified hemibody constructs. Purified hemibodies were analyzed for aggregation and purity using the AKTApure system

and Superdex 200 5/150 Increase size exclusion column. As molecular weight standard protein beta-amylase (200 kDa) and carbonic anhydrase (29 kDa) was used. mAU, milli-arbitrary units; ml_, milli-litre.

FIG. 24: Subunit composition of KiHss hemibody constructs

Construct VL antiEpcam: VL UCHT1 fused to a Fc hole chain (having the sequence shown in SEQ ID No.:2), and a binding moiety consisting of a Darpin antiEpCam Ec4 fused to a Fc knob chain (having the sequence shown in SEQ ID No.:4). Construct VH anti Epcam: VH UCHT1 fused to a Fc hole chain (having the sequence shown in SEQ ID No.:1), and a binding moiety consisting of a Darpin antiEpCam Ec4 fused to a Fc knob chain (having the sequence shown in SEQ ID No.:4). Construct VL antiEGFR: VL UCHT1 fused to a Fc hole chain (having the sequence shown in SEQ ID No.:2), and a binding moiety consisting of a camelid single domain antibody VHH antiEGFR 9G8 fused to a Fc knob chain (having the sequence shown in SEQ ID No.:5). Construct VH antiEGFR: comprising a VL UCHT1 fused to a Fc hole chain (having the sequence shown in SEQ ID No.:2), and a binding moiety consisting of a camelid single domain antibody VHH antiEGFR 9G8 fused to a Fc knob chain (having the sequence shown in SEQ ID No.:5). Construct VL antiHer2: comprising a VL UCHT1 fused to a Fc hole chain (having the sequence shown in SEQ ID No.:2), and a binding moiety consisting of a scFv antiHerT rastuzumab fused to a Fc knob chain (having the sequence shown in SEQ ID No.:6). Construct VL antiSLAM7: VL UCHT1 fused to a Fc knob chain (SEQ ID No.3), and a binding moiety consisting of an scFv antiSLAMF7 Elotuzumab fused to an Fc hole chain (SEQ ID No.9). Construct VH anti CD38: VH UCHT1 fused to a Fc hole (SEQ ID No.10), and a binding moiety consisting of an scFv antiCD38 Fc knob (SEQ ID No.11).

FIG. 25: Architecture of the used recombinant proteinaceous binding molecule (KiHss hemibody constructs) FIG. 25A shows a proteinaceous recombinant binding molecule of the invention having the binding moiety consisting of a Darpin antiEpcam Ec4 fused to a Fc knob chain, and a Variable fragment of either the light chain or the heavy chain of the CD3 specific antibody clone UCHT1 fused to a Fc hole chain, wherein the

Variable fragment of either the light chain or the heavy chain and the binding moiety are arranged at the N-terminus of the polypeptide. FIG. 25B shows a proteinaceous recombinant binding molecule of the invention having the binding moiety consisting of a VHH antiEGFR 9G8 fused to a Fc knob chain, and a Variable fragment of either the light chain or the heavy chain of the CD3 specific antibody clone UCHT1 fused to a Fc hole chain, wherein the

Variable fragment of either the light chain or the heavy chain and the binding moiety are arranged at the N-terminus of the polypeptide. FIG. 25C shows a proteinaceous recombinant binding molecule of the invention having the binding moiety consisting of a scFv antiHerT rastuzumab fused to a Fc knob chain, and a Variable fragment of either the light chain or the heavy chain of the CD3 specific antibody clone UCHT 1 fused to a Fc hole chain, wherein the Variable fragment of either the light chain or the heavy chain and the binding moiety are arranged at the N-terminus of the polypeptide. FIG. 25D shows a proteinaceous recombinant binding molecule of the invention having the binding moiety consisting of a scFv antiSlamF7 Elotuzumab fused to a Fc hole chain, and a Variable fragment of either the light chain or the heavy chain of the CD3 specific antibody clone UCHT1 fused to a Fc knob chain, wherein the Variable fragment of either the light chain or the heavy chain and the binding moiety are arranged at the N-terminus of the polypeptide. FIG. 25E shows a proteinaceous recombinant binding molecule of the invention having the binding moiety consisting of a scFv antiCD38 fused to a Fc knob chain, and a Variable fragment of either the light chain or the heavy chain of the CD3 specific antibody clone UCHT1 fused to a Fc hole chain, wherein the Variable fragment of either the light chain or the heavy chain and the binding moiety are arranged at the N-terminus of the polypeptide. The Fc fragment of all the constructs consists of a hinge region (also referred herein as “linker”), CH2 and “CH3 knob” or “CH3 hole” chain. This structure ensures that only one pairing of “CH3 knob” with “CH3 hole” is possible. The hinge region contains two cysteines forming one or more disulfide bridges. To improve stability of the knob into hole heterodimer a serine at position 354 in the knob Fc chain and a tyrosine at position 349 in the hole Fc chain in hole were substituted for cysteine enabling the formation of a stabilizing disulfide bridge. To silence Fc effector function the amino acid substitutions L234A, L235A and N297A were introduced in the knob and hole Fc chain.

FIG. 26: Possible architecture of a recombinant proteinaceous binding molecule. Proteinaceous recombinant binding molecule having the binding moiety fused to the C-terminus (shown in FIG. 26A) or N-terminus (shown in FIG. 26B) of the Fc hole or Fc knob chain of the Fc fragment and the variable domain of either the light chain or the heavy chain fused to the C-terminus (FIG. 26A) or N-terminus (FIG. 26B) of the Fc hole or Fc knob chain. The Fc fragment consists of a hinge region, CH2 and “CH3 knob” or “CH3 hole” chain, also referred to as “Fc knob chain” or “Fc hole chain”. This structure ensures that only one pairing of “CH3 knob” with “CH3 hole” is possible. The hinge region can contain two, three or four cystines forming one or more disulfide bridges.

FIG. 27: Possible architecture of a recombinant proteinaceous binding molecule. Proteinaceous recombinant binding molecule having the binding moiety fused to the C- or N-terminus of the Fc knob chain (FIG. 27A) of the Fc fragment or to the C- or N-terminus of the Fc hole chain (FIG. 27B) and the variable domain of either the light chain or the heavy chain is fused to the C-terminus or N-terminus of the Fc hole chain (FIG. 27A) or to the C- or N-terminus of the Fc knob chain of the Fc fragment (FIG. 27B). The Fc fragment consists of a hinge region, CH2 and “CH3 knob” or “CH3 hole” chain, also referred to as “Fc knob chain” or “Fc hole chain”. This structure ensures that only one pairing of “CH3 knob” with “CH3 hole” is possible. The hinge region can contain two, three or four cystines forming one or more disulfide bridges.

FIG. 28: Possible architecture of a recombinant proteinaceous binding molecule. Proteinaceous recombinant binding molecule having a first and a second binding moiety fused to the C- and N-terminus of the Fc knob chain (FIG. 28A) or of the Fc hole chain (FIG. 28B) of the Fc fragment, and the variable domain of either the light chain or the heavy chain is fused to the C- or N-terminus of the Fc hole chain (FIG. 28A) or Fc knob chain (FIG. 28B) of the Fc fragment. The Fc fragment consists of a hinge region, CH2 and “CH3 knob” or “CH3 hole” chain, also referred to as “Fc knob chain” or “Fc hole chain”. This structure ensures that only one pairing of “CH3 knob” with “CH3 hole” is possible. The hinge region can contain two, three or four cystines forming one or more disulfide bridges.

FIG. 29: Possible architecture of a recombinant proteinaceous binding molecule. Proteinaceous recombinant binding molecule having a binding moiety fused to the C- or N-terminus of the Fc hole chain of the Fc fragment (FIG. 29A), (FIG. 29B), and a first and a second variable domain of either the light chain or the heavy chain fused to the C- and N-terminus of the Fc knob chain of the Fc fragment (FIG. 29A), (FIG. 29B). The Fc fragment consists of a hinge region, CH2 and “CH3 knob” or “CH3 hole” chain, also referred to as “Fc knob chain” or “Fc hole chain”. This structure ensures that only one pairing of “CH3 knob” with “CH3 hole” is possible. The hinge region can contain two, three or four cystines forming one or more disulfide bridges.

FIG. 30: Possible architecture of a recombinant proteinaceous binding molecule. Proteinaceous recombinant binding molecule having a binding moiety fused to the C-terminus (FIG. 30A) or N-terminus (FIG. 30B) of the Fc knob chain of the Fc fragment, and a first and a second variable domain of either the light chain or the heavy chain fused to the C- and N-terminus of the Fc hole chain Fc fragment. The Fc fragment consists of a hinge region, CH2 and “CH3 knob” or “CH3 hole” chain, also referred to as “Fc knob chain” or “Fc hole chain”. This structure ensures that only one pairing of CH3 “knob” with CH3 “hole” is possible. The hinge region can contain two, three or four cystines forming one or more disulfide bridges.

FIG. 31: Possible architecture of a recombinant proteinaceous binding molecule. Proteinaceous recombinant binding molecule having two binding moieties fused to the C- and N-terminus of the Fc knob chain (FIG. 31 A) or of the Fc hole chain (FIG. 31 B) of the Fc fragment, and a first and a second variable domain of either the light chain or the heavy chain fused to the C- and N-terminus of the Fc hole chain (FIG. 31 A) or of the Fc knob chain (FIG. 32B) of the Fc fragment. The Fc fragment consists of a hinge region, CH2 and “CH3 knob” or “CH3 hole” chain, also referred to as “Fc knob chain” or “Fc hole chain”. This structure ensures that only one pairing of “CH3 knob” with “CH3 hole” is possible. The hinge region can contain two, three or four cysteines forming one or more disulfide bridges. The Fc fragment consists of a hinge region, CH2 and CH3 “knob” or “CH3 hole” domain. This structure ensures that only one pairing of CH3 “knob” with CH3 “hole” is possible. The hinge region can contain two, three or four cystines forming one or more disulfide bridges.

FIG. 32: Elimination of myeloma cells in vivo: KiHss Hemibodies successfully targeted the multiple myeloma associated antigens CD38 and SLAMF7 in vivo. FIG. 32A For in vivo analysis of hemibodies, 2^{c}10*6 luciferase-positive MM. 1S cells were injected per animal intravenously (i.v.) in immunodeficient NOD SCID H2rg−/− (NSG) mice. Twenty one (21) days after tumor cell transplantation 3^{c}10*6 CD8 positive T cells purified from human PBMCs were injected per animal intravenously (i.v.) followed by two subcutaneous (s.c.) applications of either 1×PBS, hemibodies addressing SLAMF7 (VL antiSLAMF7) or CD38 (VH antiCD38) or the combination of both hemibodies on day 22 and 26 at a concentration of 8 pg/mouse and injection. The injection volume was 200 pl_per sample and injection. Tumor growth of luciferase-positive MM.1S cells was monitored on day 21, 28 and 35 by I VIS Lumina XR Real-Time Bioluminescence Imaging. One mouse in the PBS group was not available for imaging on day 28 due to inefficient anesthesia. FIG. 32B shows survival of mice treated with either control solution (PBS), one single KiHSS hemibody, or two KiHss hemibody forming a heterodimer in vivo by association via their VH and VL (“Kombi VH/VL”) was monitored daily until day 98. As can be seen in FIG. 32B, a higher survival rate was observed in mice treated with the combination of two KiHss hemibodies, forming in vivo a heterodimer on a target cell by association of their respective VH and VL, as described herein. This experiment shows that in particular such tri-specific heterodimers (formed by association of two single KiHss molecules) provide for a high specificity of T-cell engaging binding molecules of the invention when targeting a tumor cell. This improved specificity should thus also reduce off-target activity of T-cell engaging recombinant proteinaceous binding molecules of the present invention.

FIG. 33: Sequences that might be included in the recombinant proteinaceous binding molecules of the invention: FIG. 33A shows SEQ ID No.1: VHUCHT1 human IgG1 Fc hole; FIG. 33B shows SEQ ID NO.2: VLUCHT1 human lgG1 Fc hole; FIG. 33C shows SEQ ID No.3: VLUCHT1 human IgGIFc knob; FIG. 33D shows SEQ ID No.4: Darpin antiEpcam Ec4 human lgG1 Fc knob; FIG. 33E shows SEQ ID No.5: VHH antiEGFR 9G8 human lgG1 Fc knob; FIG. 33F shows SEQ ID No.6. scFv antiHer2 Trastuzumab human IgG1 Fc knob Mw 53.152 kDa; FIG. 33G shows SEQ. ID NO. 9: scFv antiSLAMF7 human IgGIFC hole; FIG. 33H shows SEQ. ID NO. 10: VHUCHT1 human IgG1 FC hole; and FIG. 331 shows SEQ. ID NO. 11: scFv antiCD38 human IgGIFC knob. FIG. 33J shows SEQ ID No.

51: Darpin antiRORI G3w human lgG1 Fc knob. In the sequences, a secretion sequence (taken from patent US20150337027A1) is shown in italic, the variable domain of either an antibody heavy chain (VH) or an antibody light chain (VL) of a second binding site are in bold italic, the GS-Hinge IgG1 linker is underlined, the CH2 domains are highlighted in light grey and the CH3 knob/hole domains are highlighted in dark grey.

FIG. 34: Serum half-life of KiHss hemibodies: FIG. 34A shows serum half-life of an hemibody in the original format as described, for example, in Banaszek et al, Nature Communications (2019) 10:5387 (https://doi.org/10.1038/s41467-019-13196-0), while FIG. 34B shows serum half-life of a KiHss hemibody of the invention. FIG. 34 shows that the KiHss hemibody of the invention has a serum half-life of at least 500 min, while the corresponding original hemibody has a serum half-life of little over 100 minutes, meaning binding molecules of the present invention have a significantly longer serum half-life.

FIG. 35: Hemibody mediated T-cell activation: The activation assay was performed with serial diluted (dilution factor 2) hemibody constructs VH antiEpcam+VL antiHer2 and VH antiRORI+VL antiHer2 starting at a concentration of 5 nM for each hemibody with Jurkat Lucia NFAT cells as effector cells and firefly luciferase expressing HCT116 cells as target cells. nM, Nanomolar; RLI, relative luminescence intensity.

FIG. 36: Hemibody mediated T-cell activation: The activation assay was performed with serial diluted (dilution factor 2) hemibody constructs VH antiEpcam÷VL antiEGFR and VH antiRORI+VL antiEGFR starting at a concentration of 5 nM for each hemibody with Jurkat Lucia NFAT cells as effector cells and firefly luciferase expressing HT29 cells as target cells. nM, Nanomolar; RLI, relative luminescence intensity.

FIG. 37: Antigen density status of used tumor cell lines HT29 and HCT116.

Cells were stained with target specific APC conjugated antibodies and subjected to flow cytometric analysis. Solid, target antigen; Line, isotype control; Bold, median target antigen signal intensity; underlined, median of isotype control signal intensity.

### DETAILED DESCRIPTION OF THE INVENTION

In order to circumvent some of the limitations of the current immunotherapic strategies, such as the specificity of T cell engaging antibodies, a novel antibody format has been developed, based on the binding of one or two tumor associated antigens simultaneously and the activation of an immune response subsequently. These so called KiHss hemibodies are based on a binding moiety, specific for a tumor-associated target, flexibly linked to a variable domain of an antibody light chain (VL) or to a variable domain of an antibody heavy chain (VH) specific for a receptor molecule, like for example the T cell co receptor CD3. Concurrent binding of two KiHss hemibodies (for example, with diverse specificity) on the cell surface of a single tumor cell enables the formation of, for example, an antiCD3 binding domain from the two subunits VL and VH. This allows the recruitment of T

cells towards the target cell, leading to an activation of the immune cells and the killing of double positive tumor cells (Banaszek A, Bumm T G P, Nowotny B, Geis M, Jacob K, Wolfl M, et al. On-target restoration of a split T cell-engaging antibody for precision immunotherapy. Nature communications 2019; 10:5387). So far, the original hemibody formats were produced with prokaryotic and eukaryotic expression systems and with the help of tags (such as His- or Strep-tag®). However, for a potential clinical development, the current production methods are insufficient, especially in terms of product yield (titre), homogeneity and quality of the product and purification. Furthermore, first experiments in mice have shown that the terminal half-life in vivo of such original hemibodies is very short (about 30 min to 1 h) and thus requires continuous infusions to maintain therapeutic concentrations in cancer patients (see, for example, the summary of product characteristics for blincyto of the EMA, available at https://www.ema.europa.eu/en/documents/product-information/blincyto-epar-product information_en.pdf). Due to the above-mentioned shortcomings, the original hemibody formats are currently not clinically available. Furthermore, the original hemibody technology alone has the advantage to reduce the therapeutic antibodies to small fragments, achieving a better tumor penetration. However, this also leads to disadvantages like the fast elimination from the body.

One advantage of the KiHss hemibody-based strategy of the present invention is the fact that compared to conventional antibody therapies, antigen combinations and not only individual antigens can be addressed therapeutically and thus a considerably higher specificity and therapeutic safety is achieved. Furthermore, by comparison with published data, the addition of Fc Fragments to the original hemibody constructs are considered to accomplish extend half-life via neonatal Fc-receptor (FcRn) binding, therefore overcoming the limitation of fast elimination from the body of the original hemibody format.

Accordingly, the inventors found that it is possible to advantageously use Fc Fragments as fusion partner for the “hemibody approach” to provide the new recombinant proteinaceous binding molecules of the present invention, also referred to as “KiHss hemibodies”. Therefore, in the present invention the binding moiety specific for a tumor-associated target and the VH or VL domain specific for a receptor molecule were linked to single chain Fc fragments, in particular, to Fc fragments modified according to the “knob-into-hole” technology. To increase the probability of heterodimer (formed by a binding moiety specific for a first antigen, defined elsewhere herein, plus a VL or VH specific for CD3 formation the sulfide stabilized knob-into-hole (herein referred to as KiHss) technology was used whereby complementary mutations were added to the CH3 domain of each Fc heavy chain leading to the recombinant proteinaceous binding molecule of the invention, also referred to as “KiHss hemibody”.

For this purpose the inventors made use of asymmetric Fc Fragments of the “knob-into hole” class (as described in U.S. Pat. No. 8,2422,47 or European patent EP2 225 280 B1, for example). The dimerization of two Fc fragments is essentially characterized by the nanomolar affinity of the CH3 domain for itself. Forming cysteine bridges in the hinge region additionally stabilize Fc-dimers. Therefore, this Invention describes the use of an asymmetric Fc fragment which is the “knob-into-hole” technology, defined elsewhere herein. Fc fragments are used as fusion partners and are altered by mutations in such a way that their molecular structure changes and the CH3 domains present a cavity, or “hole” and the other CH3 domains a bulge (“knob”). This means that, according to the key-lock principle, only CH3 domains with a “hole” can interact with and bind to those that have a “knob”. Interactions of structurally identical CH3 domains (knob-knob or holehole), as is the case in the wild type, are thus hindered excluded. This asymmetric pairing of Fc fragments allows the generation of bispecific molecules that can, for example, simultaneously bind an antigen and CD3 on the surface of T cells or CD40 on the surface of antigen presenting cells (APC), including dendritic cells, B cells and macrophages. CD3 (cluster of differentiation 3), defined elsewhere herein, is a protein complex and T cell co-receptor that is required to activate the cytotoxic T cell (CD8+ T cells) and T helper cells (CD4+ T cells). CD40 is (cluster of differentiation 40) is a costimulatory protein found and constitutively expressed on antigen-presenting cells (APC) that is required for activation of APCs.

The use of “Fc hole-chain” and “Fc knob-chain” as fusion partner has also the advantage of allowing control the Fc-Fc pairings and therefore avoid altered valency, which means that upon dimerization, the heterodimer will not be able to bind even more antigens and this will avoid cytotoxic hyperactivation of T cells. It is noted here that each of the CH2 domain and the CH3 domain that is used in the present invention is preferably an IgG CH2 or CH3 domain (the constant domains can be of any of the four IgG subclasses, i.e. a lgG1, lgG2, lgG3 or an lgG4 constant domain). While it is preferred that the CH2 domain and the CH3 domain of the “Fc-hole chain” are of the same subclass as the CH2 domain and the CH3 domain of the “Fc knob-chain”, it is also possible to use in binding molecules of the invention CH2 and CH3 domains of different IgG subclasses, for example, to use a lgG1 CH2 and CH3 domain for the “Fc-hole chain” and an lgG2 CH2 and CH3 domain for the “Fc knob-chain”. It is also possible to use in one chain, for example, the “Fc-hole chain”, an lgG2 CH2 domain and an lgG1 CH3 domain.

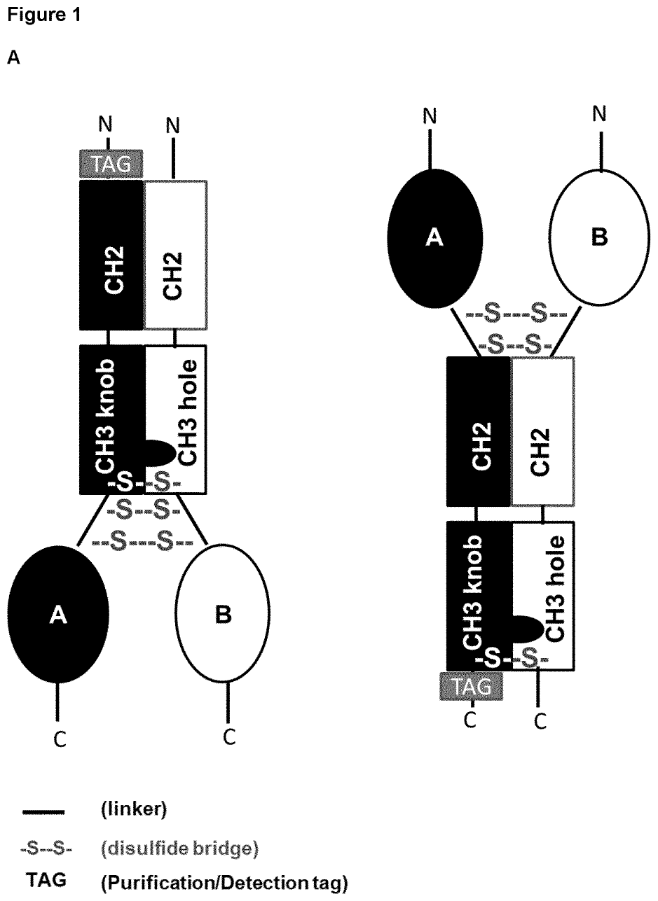

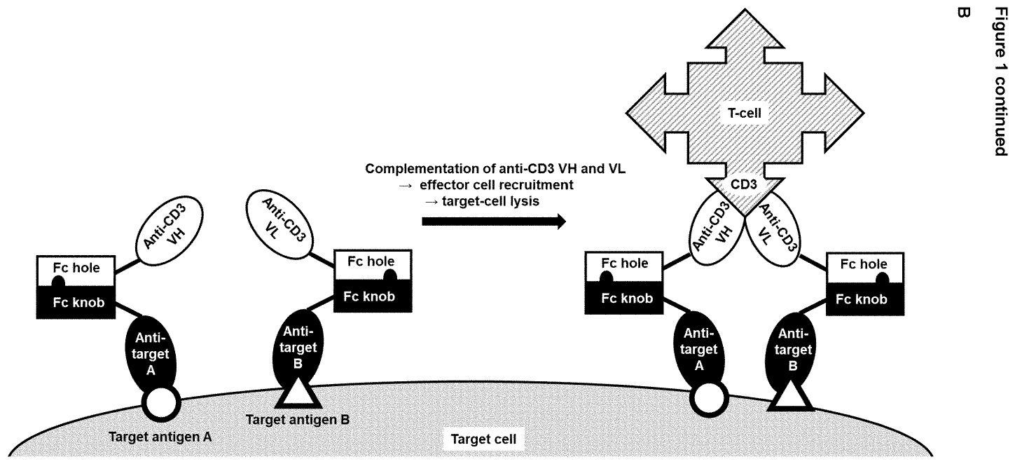

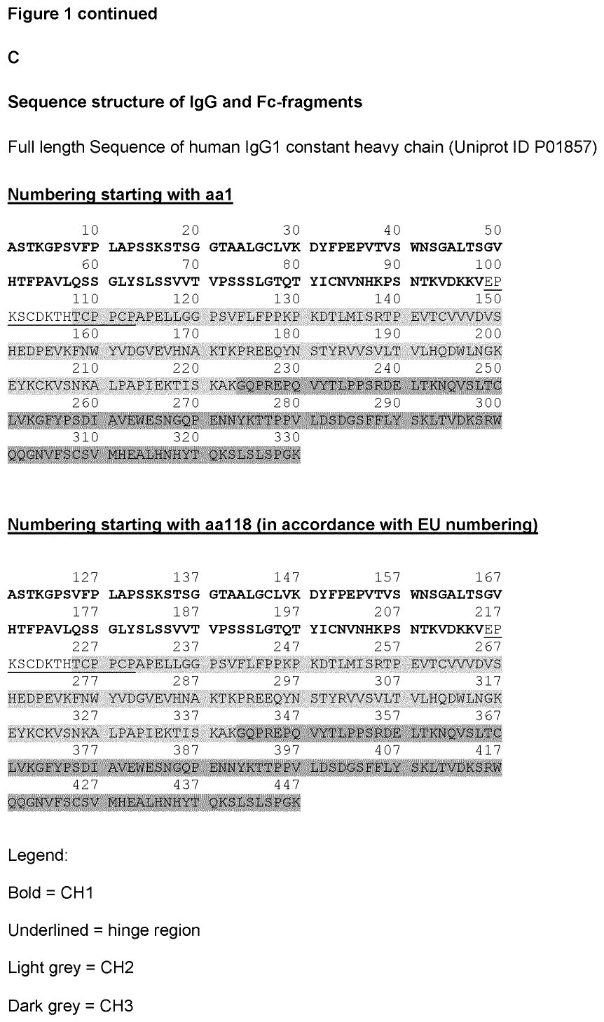

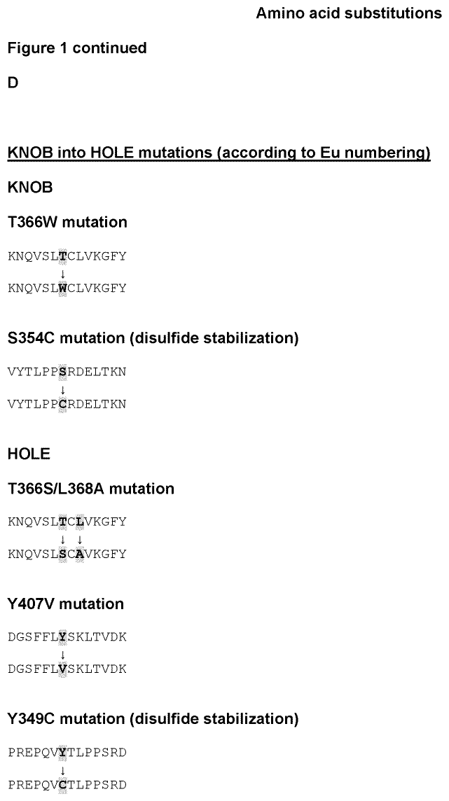

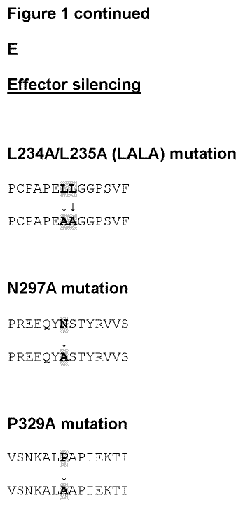

Through the replacement of small amino side chains with larger (bulkier) ones (here: T366W according to the EU numbering)—all the amino acid positions used herein are the positions according to the EU numbering. For IgG EU numbering see www.imgt.org/AMGTScientificChart/Numbering/Hu_IGHGnber.html. See also FIG. 1C-E-, knobs were created. Said immunoglobulin CH3 domain is also referred to herein as “CH3

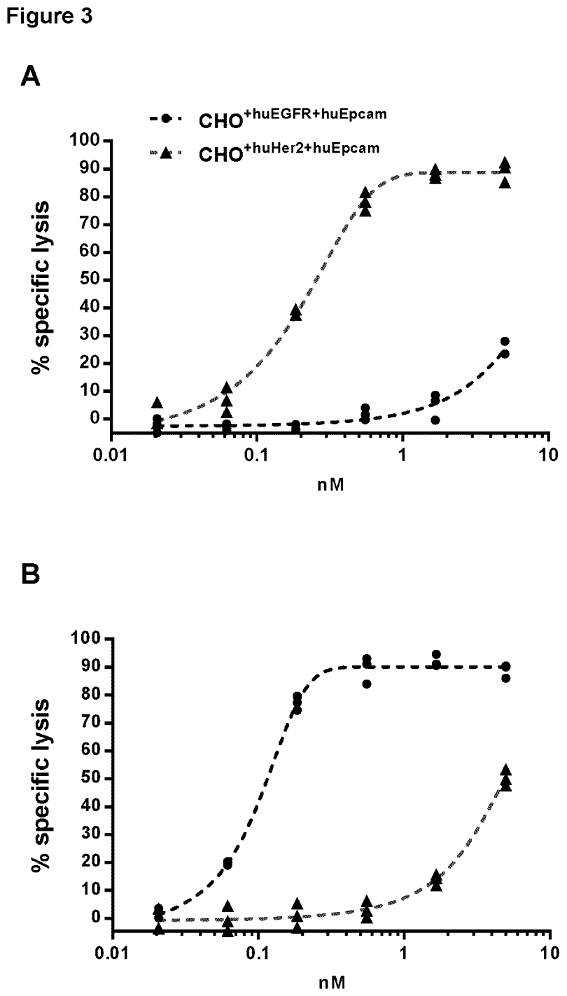

knob-chain”. The construction of holes occurred via a substitution of large side chains with smaller ones (here: T366S/L368A/Y407V according to the EU numbering). As used herein said immunoglobulin CH3 domain comprising said mutation is referred to as “CH3 hole-chain” (Ridgway J B B, Presta L G, Carter P. ‘Knobs-into-holes’ engineering of antibody CH3 domains for heavy chain heterodimerization. Protein Engineering 1996:617-21). As used herein, the heavy chain of the Fc fragment comprising the CH3-hole chain is referred to as “Fc hole-chain”, and the heavy chain of the Fc fragment comprising the CH3-knob chain is referred to as “Fc knob-chain”. As used herein, the term “CH3 knob chain” and “Fc knob-chain” can be used interchangeably. Similarly, the terms “CH3 hole-chain” and “Fc hole-chain” can be used interchangeably. These used T366W-T366S:L368A:Y407V mutations have been reported to form stable heterodimers (Atwell, S, al e. Stable Heterodimers from Remodeling the Domain Interface of a Homodimer using a Phage Display Library. J. Mol. Biol. 1997.26-35). To further increase the stability, two cysteine mutations were added (CH3 knob chain: S354C, CH3 hole chain: Y349C) enabling the formation of a disulfide bridge. To avoid Fc-mediated target cell killing through single hemibodies, three additional mutations were added to the CH2 region: L234A/L235A and N297A. These mutations led to effector silencing and aglycosylation respectively and reduced the antibody interaction with Fc receptors in particular FcyRs and C1q. An Fc receptor (herein also referred to as “FcR”) is a protein found on the surface of, among others, B lymphocytes, follicular dendritic cells, natural killer cells, macrophages, neutrophils, eosinophils, basophils, human platelets, and mast cells—that contribute to the protective functions of the immune system. The name is derived from its binding specificity for the Fc (fragment crystallizable) region of antibodies, defined elsewhere herein. Fc receptors bind to antibodies that are attached to infected cells or invading pathogens. Their activity stimulates phagocytic or cytotoxic cells to destroy microbes, or infected cells by antibody-mediated phagocytosis or antibody-dependent cell-mediated cytotoxicity. This ensured that only specific scFv mediated tumor cell killing and no ADCC or CDC occurred (Wang X, Mathieu M, Brezski R J. IgG Fc engineering to modulate antibody effector functions. Protein & cell 2018; 9:63-73). VH or VL parts of an anti CD3 effector and the anti-target (for example an scFvs, or a Darpin) are fused to either a Fc-knob or Fc-hole single chain via a truncated IgG1 hinge region also referred herein as “linker”. These hinge regions also carry cysteines and form one or more disulfide bridges. One KiHss hemibody may, for example, comprise a binding moiety with specificity against one tumor associated target antigen (also referred herein to as “anti-target”) and a VH or VL with half of a binding site for a for a receptor molecule, such as T cell receptor CD3. (CD3 effector). The combination of two KiHss hemibodies with diverse anti-target specificities via the combination of the VH of the first KiHss hemibody and the VL of the second KiHss hemibody may form an effective “heterodimer” or anti-effector/anti-target molecule (for example an
aCD3/scFv) and recruit cytotoxic T cells after target cell binding (see FIG. 1). The combination of two KiHss hemibodies thereby forms the heterodimeric recombinant proteinaceous binding molecule defined elsewhere herein, wherein each KiHss hemibody constitutes a monomer. In this context, a “target cell” may be a tumor cell, in particular, the target cell may be a cell expressing a tumor associated antigen selected from the group consisting of SlamF7, CD10, CD19, CD20, CD21, CD22, CD25, CD30, CD33, CD34, CD37, CD38, CD44v6, CD45, CDw52, CD70, CD117, CD123, CD133, CD135, CD138, CD140a, CD140b, CD171, CD309 CSPG4, Muc-1,Muc-16 Erb-B1, Erb-B2, Erb-B3, EGFRvlll, Folate receptor, PSMA, PSCA, PSA, VEGFR2, TAG-72, HLA-DR, IGFR, IL3R, fibroblast activating protein (FAP), CEA, EpCAM, CIaudin6, CLL-1, EphAIO, G250, BB2, gp100, NY-ESO-1, LAGE-1, MAGE-A1, MAGE-A3, P-Cadherin, N-Cadherin, E-Cadherin, HLA-DP, HLA-A2, CCR4, CXCR3, FGFR1, GPC3, GPA33, GD2, BCMA, ROR , TIM-3, Mesothelin (MSLN). Furthermore, the addition of a Fc fragment to those hemibodies may allow an extended half time life via binding to the neonatal Fc-receptor (FcRn). FIG. 34 shows that the KiHss hemibodies of the present invention display an improved half-life (measured as time needed for elimination of the KiHss hemibodies from the body). In particular, FIG. 34, shows that the KiHss hemibody pair scFv antiSLAMF7 human IgG1 FC hole (SEQ. ID No. 9) and Vl_dil_2k human IgGIFC knob (SEQ. LD No. 30) of the invention has a serum half-life of at least 500 min, while the corresponding original hemibody has a serum half-life of little over 100 minutes, meaning binding molecules of the present invention have a significantly longer serum half-life\hat{ }Thus, without being bound by theory, the KiHss hemibody constructs of the present invention provide for an improved efficacy to engage T-cells towards tumor, e.g., due to their longer serum half-life which requires less injections, compared to hemibodies in the original format which may require daily administration in order to target multiple myeloma associated antigens as, e.g., shown in FIG. 3D of Geis et al (see, e.g., Geis, M., Nowotny, B., Bohn, M D. et al. Combinatorial targeting of multiple myeloma by complementing T cell engaging antibody fragments. Commun Biol 4, 44 (2021) which is incorporated herein by reference).

Accordingly, the present invention relates to a recombinant proteinaceous binding molecule. As used herein, the term “recombinant” refers to the alteration of genetic material by human intervention. Typically, the term recombinant refers to the manipulation of DNA or RNA in a virus, cell, plasmid or vector by molecular biology (recombinant DNA technology) methods, including cloning and recombination. A recombinant cell, polypeptide, or nucleic acid can be typically described with reference to how it differs from a naturally occurring counterpart (the “wild-type”). A “recombinant proteinaceous binding molecule” as used herein may refer to a proteinaceous binding molecule, that has been genetically altered to comprise an amino acid sequence which is not found in nature. This term, as used herein, also refers to a proteinaceous binding molecule which is artificially expressed in cell systems that naturally do not produce the molecule, or do not produce the molecule at high levels.

In particular, the recombinant proteinaceous binding molecule of the present invention may comprise: a) a first binding moiety, capable of binding an antigen, having a first binding site for a first antigen, b) a variable domain of either an antibody light chain or an antibody heavy chain of a second binding site for a second antigen, c) an Fc fragment comprising a first and a second heavy chain wherein the first and the second heavy chain each comprise one immunoglobulin CH2 domain and one immunoglobulin CH3 domain, wherein the CH3 domain of the first heavy chain and the CH3 domain of the second heavy chain each meet each other an at interface, wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and wherein the variable domain of either the antibody light chain or the antibody heavy chain and the first binding moiety are linked via the Fc fragment.

By “linked” herein is meant that the binding moiety (for example a target specific scFvs as defined elsewhere herein) and the variable domain of an antibody light chain or the variable domain of an antibody heavy chain (for example, the VH or VL domain specific for CD3 as defined elsewhere herein) were fused to, respectively, the first heavy chain of the Fc fragment and the second heavy chain of the Fc fragment, wherein the first and second heavy chain of the Fc fragment dimerize via the knob-in-hole technology further stabilized by the disulfide bridge (as defined herein above). In particular, the binding moiety and the variable light chain or the variable heavy chain may be linked respectively to the first heavy chain of the Fc fragment and the second heavy chain of the Fc fragment via a peptide linker sequence (or “hinge regions” defined elsewhere herein) (see FIGS. 25 to 31). The peptide linker can be any flexible linker known in the art, for example, made from glycine and serine residues. It is also possible to additionally stabilize the domain association between the VH and the VL domain by introducing disulfide bonds into conserved framework regions.

Moreover, the recombinant proteinaceous binding molecule may further comprise an affinity tag, such as a His6-tag. Affinity tags such as the Strep-tag® or Strep-tag® II

(Schmidt, T. G. M et al. (1996) J. Mol. Biol. 255, 753-766), the myc-tag, the FLAG™-tag, the His6-tag or the HA-tag allow easy detection and also simple purification of the recombinant proteinaceous binding molecule.

With the term “binding moiety” as used herein it is meant a part or functional group of a molecule. In the context of the present invention, a binding moiety may be also referred to as “anti-target”. In particular, in the context of the invention a binding moiety is a functional part of the recombinant proteinaceous binding molecule which is able to bind an antigen and has a first binding site for a first antigen. The “binding site(s)” (paratope) of a recombinant proteinaceous binding molecule as defined herein refers to the portion of the recombinant proteinaceous binding molecule that may specifically bind to/interact with an epitope. The term “epitope”, also known as the “antigenic determinant”, refers to the portion of an antigen to which an antibody, a recombinant proteinaceous binding molecule or T-cell receptor specifically binds, thereby forming a complex. Thus, the term “epitope” includes any molecule or protein determinant capable of specific binding to an immunoglobulin or T-cell receptor. The binding site(s) (paratope) of a recombinant proteinaceous binding molecule described herein may specifically bind to/interact with conformational or continuous epitopes, which are unique for the target structure. Epitopic determinants usually consist of chemically active surface groupings of molecules such as amino acids or sugar side chains and usually have specific three-dimensional structural characteristics, as well as specific charge characteristics. In some examples, epitope determinants include chemically active surface groupings of molecules such as amino acids, sugar side chains, phosphoryl, or sulfonyl, and, in certain examples, may have specific three-dimensional structural characteristics, and/or specific charge characteristics. With regard to polypeptide antigens a conformational or discontinuous epitope is characterized by the presence of two or more discrete amino acid residues, separated in the primary sequence, but assembling to a consistent structure on the surface of the molecule when the polypeptide folds into the native protein/antigen (Sela, M., Science (1969) 166, 1365-1374; Laver, W. G., et al. Cell (1990) 61, 553-556). The two or more discrete amino acid residues contributing to the epitope may be present on separate sections of one or more polypeptide chain(s). These residues come together on the surface of the molecule when the polypeptide chain(s) fold(s) into a three-dimensional structure to constitute the epitope. In contrast, a continuous or linear epitope consists of two or more discrete amino acid residues, which are present in a single linear segment of a polypeptide chain. As an illustrative example, a “context-dependent” CD3 epitope refers to the conformation of said epitope. Such a context-dependent epitope, localized on the epsilon chain of CD3, can only develop its correct conformation if it is embedded within the rest of the epsilon chain and held in the right position by heterodimerization of the epsilon chain with either CD3 gamma or delta chain. In contrast thereto, a context-independent CD3 epitope

may be an N-terminal 1-27 amino acid residue polypeptide or a functional fragment thereof of CD3 epsilon. Generally, epitopes can be linear in nature or can be a discontinuous epitope. Thus, as used herein, the term “conformational epitope” refers to a discontinuous epitope formed by a spatial relationship between amino acids of an antigen other than an unbroken series of amino acids. The term “epitope” also includes an antigenic determinant of a hapten, which is known as a small molecule that can serve as an antigen by displaying one or more immunologically recognized epitopes upon binding to larger matter such as a larger molecule e.g. a protein.

An antibody or recombinant proteinaceous binding molecule/fragment is said to specifically bind to an antigen when it recognizes its target antigen in a complex mixture of proteins and/or macromolecules. Antibodies or recombinant proteinaceous binding molecules according to the invention are said to “bind to the same epitope” if they cross-compete so that only one antibody or recombinant proteinaceous binding molecule can bind to the epitope at a given point of time, i.e. one prevents the binding or modulating effect of the other.

The term “specific” in this context, or “specifically recognizing”, also used as “directed to”, means in accordance with this invention that the recombinant proteinaceous binding molecule is capable of specifically interacting with and/or binding to at least two, e.g. at least three or at least four amino acids of an epitope but does not essentially bind to another epitope or antigen. Such binding may be exemplified by the specificity of a “lock-and-key-principle”. Specific binding is believed to be affected by specific motifs in the amino acid sequence of the binding region of a recombinant proteinaceous binding molecule, and the recombinant proteinaceous binding molecule and the epitope or the antigen bind to each other as a result of their primary, secondary or tertiary structure as well as the result of secondary modifications of said structure. The specific interaction of the epitope/antigen-interaction-site with its specific epitope/antigen may result as well in a simple binding of said site to the antigen. Moreover, the specific interaction of the antigen-interaction-site with its specific epitope/antigen may alternatively result in the initiation of a signal, such as for instance due to the induction of a change of the conformation of the antigen or an oligomerization of the antigen.

Typically, binding specificity is the ability of a binding molecule to discriminate between similar or even dissimilar antigens. As used herein, a proteinaceous binding molecule of the disclosure “specifically binds” a target if it is able to discriminate between that target and one or more reference targets, since binding specificity is not an absolute, but a relative property. “Specific binding” can be determined, for example, in accordance with Western blots, ELISA-, RIA-, ECL-, IRMA-tests, FACS, IHC and peptide scans.

The recombinant proteinaceous binding molecule of the present invention further comprises a Variable domain of either an antibody light chain (VL) or an antibody heavy chain (VH) of a second binding site for a second antigen. In the context of the present invention, this means that the Variable domain of either the antibody light chain or antibody heavy chain have one half of a second binding site for a second antigen, therefore being an incomplete binding site which, as disclosed elsewhere herein, will form a complete second binding site by association to a single VH or VL domain of a second monomer (KiHss hemibody)); the complete second binding site is also referred to as “anti-effector” since it binds to an antigen present on the effector T-cells, such as CD3. The term “variable” refers to the portions of the immunoglobulin domains that exhibit variability in their sequence and that are involved in determining the specificity and binding affinity of a particular antibody (i.e., the “variable domain(s)”). Variability is not evenly distributed throughout the variable domains of antibodies, it is concentrated in sub-domains of each of the heavy and light chain variable regions. The terms “VH” and “VL” are used herein to refer to the heavy chain variable domain and light chain variable domain respectively of an immunoglobulin. An immunoglobulin light or heavy chain variable region consists of a “framework” region interrupted by three hypervariable regions. Thus, the term “hypervariable region” refers to the amino acid residues of an antibody which are responsible for antigen binding. The hypervariable region includes amino acid residues from a “Complementarity Determining Region” or “CDR”. There are three heavy chains and three light chain CDRs (or CDR regions) in the variable portion of an immunoglobulin. Thus, “CDRs” as used herein refers to all three heavy chain CDRs (CDRH1, CDRH2 and CDRH3), or all three light chain CDRs (CDRH, CDRL2 and CDRL3) or both all heavy and all light chain CDRs, if appropriate. Three CDRs make up the binding character of a light chain variable region and three make up the binding character of a heavy chain variable region. CDRs determine the antigen specificity of an immunoglobulin molecule and are separated by amino acid sequences that include scaffolding or framework regions. The exact definitional CDR boundaries and lengths are subject to different classification and numbering systems. The structure and protein folding of the antibody may mean that other residues are considered part of the antigen binding region and would be understood to be so by a skilled person. CDRs provide the majority of contact residues for the binding of the immunoglobulin to the antigen or epitope. In preferred embodiments of the invention, the recombinant proteinaceous binding molecule comprises a variable domain of either an antibody heavy chain (VH) or of an antibody light chain (VL) of a second binding site for a second antigen, wherein the VH or VL may comprise one part, or “one half”, of a binding site for a T cell receptor, for example CD3. As defined herein above, the combination of two KiHss hemibodies (or monomers, defined elsewhere herein) each having either the VH or VL with half of a binding site for a for a T cell receptor may form an effective anti-effector/anti-target molecule and recruit cytotoxic T cells after target cell binding (see FIG. 1). Said anti-effector/anti-target corresponds to the heterodimeric recombinant proteinaceous binding molecule defined elsewhere herein, formed by two KiHss hemibodies (or monomers). The anti effector may be for example an anti-CD3 and the anti-target may be an antiEpcam, antiEGFR, antiHer2 Trastuzumab, antiSLAMF7, antiCD38, antiRORI. CD3 (cluster of differentiation 3) is a protein complex and part of the T cell receptor (TCR). The T cell receptor (TCR) is a particular receptor that is present on the cell surface of T cells, i.e. T lymphocytes and it is involved in activating both the cytotoxic T cell (CD8+ T cells) and T helper cells (CD4+ T cells). In vivo the T cell receptor exists as a complex of several proteins. The T cell receptor generally has two separate peptide chains, typically T cell receptor alpha and beta (TCRa and TOEb) chains, on some T cells T cell receptor gamma and delta (TCRy and TCRb). The other proteins in the complex are the CD3 proteins: CD3sy and CD3sb heterodimers and, most important, a 003z homodimer, which has a total of six ITAM motifs. The ITAM motifs on the 003z can be phosphorylated by Lck and in turn recruit ZAP-70. Lck and/or ZAP-70 can also phosphorylate the tyrosines on many other molecules, not least CD28, LAT and SLP-76, which allows the aggregation of signalling complexes around these proteins.

The recombinant proteinaceous binding molecule of the present invention further comprises an Fc fragment. The fragment crystallizable region (Fc region) is the tail region of an antibody that interacts with cell surface receptors called Fc receptors and some proteins of the complement system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. Fc binds to various cell receptors and complement proteins. In this way, it mediates different physiological effects of antibodies (detection of opsonized particles; cell lysis; degranulation of mast cells, basophils, and eosinophils; and other processes). The Fc part mediates the effector function of antibodies, e.g. the activation of the complement system and of Fc-receptor bearing immune effector cells, such as NK cells. In human IgG molecules, the Fc region is generated by papain cleavage N-terminal to Cys226. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody molecule, or by recombinantly engineering the nucleic acid encoding a heavy chain of the antibody molecule. Accordingly, a composition of intact antibodies may include antibody populations with all K447 residues removed, antibody populations with no

K447 residues removed, and antibody populations having a mixture of antibodies with and without the K447 residue. The term “Fc region” or “Fc fragment” is used herein to define a C-terminal or the N-terminal region of the recombinant proteinaceous binding molecule of the invention and it may include native-sequence Fc regions and variant Fc regions. Suitable native-sequence Fc regions for use in the recombinant proteinaceous binding molecules of the invention include mammalian, e.g. human or murine, lgG1, IgG2 (lgG2A, lgG2B), lgG3 and IgG4. The Fc region contains two or three constant domains, depending on the class of the antibody. In embodiments where the immunoglobulin is an IgG the Fc region has a CH2 and a CH3 domain.

In the context of the present invention, the binding moiety (having a first binding site) of the recombinant proteinaceous binding molecule of the invention may be an antibody fragment. Generally, an “antibody fragment” refers to the fragment antigen-binding (Fab), or the fragment crystallizable (Fc), which are two regions of a full antibody molecule (or immunoglobulin (Ig). However, an “antibody fragment” as used herein refers to a wide variety of antibody fragments which has been developed as alternative platforms to IgGs. The most significant advantages to antibody fragments include size, manufacturing, tissue penetration, and ability to concatenate to generate multi-specificity (Hanahan D, Weinberg R A. Hallmarks of cancer: the next generation. Cell 2011 144:646-74). In preferred embodiments of the invention, the antibody fragment (constituting the binding moiety having a first binding site for a first antigen of the recombinant proteinaceous binding molecule of the invention) may be an scFv fragment, a F(ab′)2 fragment, an Fv fragment, or a camelid single domain antibody, as defined elsewhere herein. Furthermore, the antibody fragment, as used herein, may be a divalent or a monovalent antibody fragment, as defined elsewere herein.

The fragment antigen-binding (Fab) is a region on an antibody that binds to antigens. It is composed of one constant and one variable domain of each of the heavy and the light chain. The variable domain contains the paratope (the antigen-binding site), comprising a set of complementarity-determining regions, at the amino terminal end of the monomer. Each arm of the Y thus binds an epitope on the antigen. The terms “Fab”, “Fab region”, “Fab portion” or “Fab fragment” as used herein are therefore to be understood to define a polypeptide that includes a VH, a CH1, a VL, and a CL immunoglobulin domain. Fab may refer to this region in isolation, or this region in the context of a recombinant proteinaceous binding molecule according to the invention, as well as a full-length immunoglobulin or immunoglobulin fragment. Typically, a Fab region contains an entire light chain of an antibody. A Fab region can be taken to define “an arm” of an immunoglobulin molecule. It contains the epitope-binding portion of that Ig. The Fab region of a naturally occurring immunoglobulin can be obtained as a proteolytic fragment by a papain-digestion. A “F(ab′)2 portion” is the proteolytic fragment of a pepsin-digested immunoglobulin. A “Fab¹ portion” is the product resulting from reducing the disulfide bonds of an F(ab′)2 portion. As used herein the terms “Fab”, “Fab region”, “Fab portion” or “Fab fragment” may further include a hinge region that defines the C-terminal end of the antibody arm. This hinge region corresponds to the hinge region found C-terminally of the CH1 domain within a full-length immunoglobulin at which the arms of the antibody molecule can be taken to define a Y. The term hinge region is used in the art because an immunoglobulin has some flexibility at this region.

The fragment crystallizable region (Fc region), as defined elsewhere herein, is the tail region of an antibody that interacts with cell surface receptors called Fc receptors and some proteins of the complement system. In the context of the present invention, the “Fc region” or “Fc fragment” or “Fc domain”, as defined elsewhere herein, is the C-terminal or the N-terminal region of the recombinant proteinaceous binding molecule of the invention. In some embodiments, The Fc region comprising the CH3-hole chain as defined elsewhere herein, may have sequence identity of at least 80%, or at least 90%, or at least 95% or at least 98%, or 100% to the sequence shown in SEQ ID NO.: 7. In further preferred embodiments The Fc region comprising the CH3 knob-chain as defined elsewhere herein, may have sequence identity of at least 80%, or at least 90%, or at least 95% or at least 98%, or 100% to the sequence shown in SEQ ID NO.: 8 (both sequences shown in Table 1). In some embodiments, the CH-3 hole chain may have a sequence identity of at least 50%, or at least 60%, or at least 70%, or at least 80%, or at least 90%, or at least 95% or at least 98%, or 100% to the sequence shown in SEQ ID NO.: 28. In some embodiments, the CH3 knob chain may have a sequence identity of at least 50%, or at least 60%, or at least 70%, or at least 80%, or at least 90%, or at least 95% or at least 98%, or 100% to the sequence shown in SEQ ID NO.: 29. the CH3 hole chain may have a sequence identity of at least 50%, or at least 60%, or at least 70%, or at least 80%, or at least 90%, or at least 95% or at least 98%, or 100% to the sequence shown in SEQ ID NO.: 30. In some embodiments, the light chain of the variable fragment (VL) of the second binding site for a second antigen fused to the CH3-hole chain may have sequence identity of at least 50%, or at least 60%, or at least 70%, or at least 80%, or at least 90%, or at least 95% or at least 98%, or 100% to the sequence shown in SEQ ID NO.:2. In some embodiments, the light chain of the variable fragment (VL) of the second binding site for a second antigen fused to the CH3-knob chain may have sequence identity of at least 50%, or at least 60%, or at least 70%, or at least 80%, or at least 90%, or at least 95% or at least 98%, or 100% to the sequence shown in SEQ ID NO.:3. In some embodiments, the heavy chain of the variable fragment (VH) of the second binding site for a second antigen fused to the CH3-hole chain may have sequence identity of at least 50%, or at least 60%, or at least 70%, or at least 80%, or at least 90%, or at least 95% or at least 98%, or 100% to the sequence shown in SEQ ID NO.: 1. In some embodiments, the heavy chain of the variable fragment (VH) of the second binding site for a second antigen fused to the CH3-hole chain may have sequence identity of at least 50%, or at least 60%, or at least 70%, or at least 80%, or at least 90%, or at least 95% or at least 98%, or 100% to the sequence shown in SEQ ID NO.: 10. Further, according to some embodiments of the invention, the binding moiety having a first binding site for a first antigen fused to the CH3-knob chain may have sequence identity of at least 50%, or at least 60%, or at least 70%, or at least 80% to the sequence shown in SEQ ID NO.:4. In some embodiments, the binding moiety having a first binding site for a first antigen fused to the CH3-knob chain may have sequence identity of at least 50%, or at least 60%, or at least 70%, or at least 80% to the sequence shown in SEQ ID NO.:5. In some other embodiments the binding moiety having a first binding site for a first antigen fused to the CH3-knob chain may have sequence identity of at least 50%, or at least 60%, or at least 70%, or at least 80% to the sequence shown in SEQ ID NO.:6. In some other embodiments the binding moiety having a first binding site for a first antigen fused to the CH3-knob chain may have sequence identity of at least 50%, or at least 60%, or at least 70%, or at least 80% to the sequence shown in SEQ ID NO.: 51. In yet further embodiments the binding moiety having a first binding site for a first antigen fused to the CH3-hole chain may have sequence identity of at least 50%, or at least 60%, or at least 70%, or at least 80% to the sequence shown in SEQ ID NO.:9. Finally, In some embodiments the binding moiety having a first binding site for a first antigen fused to the CH3-knob chain may have sequence identity of at least 50%, or at least 60%, or at least 70%, or at least 80% to the sequence shown in SEQ ID NO.: 11.

The term “antibody fragment” may also refer to an “Fv” or “Fv fragment”, which consists of only the VL and VH domains of a “single arm” of an immunoglobulin. Thus an “Fv” is the minimum antibody fragment which contains a complete antigen-recognition and binding site. A “two-chain” Fv fragment consists of a dimer of one heavy- and one light-chain variable domain in tight, non-covalent association. A “single-chain” Fv fragment (scFv) includes a VH and a VL domain of an immunoglobulin, with these domains being present in a single polypeptide chain in which they are covalently linked to each other by a flexible peptide linker. Typically, in a scFv fragment the variable domains of the light and heavy chain associate in a dimeric structure analogous to that in a two-chain Fv species. In single chain Fv fragments, it is possible to either have the variable domain of the light chain arranged at the N-terminus of the single polypeptide chain, followed by the linker and the variable domain of the heavy chain arranged at the C-terminus of the polypeptide chain or vice versa, having the variable domain of the heavy chain arranged on the N-terminus and the variable domain of the light chain at the C-terminus with the peptide linker arranged inbetween. The peptide linker can be any flexible linker known in the art, for example, made from glycine and serine residues. It is also possible to additionally stabilize the domain association between the VH and the VL domain by introducing disulfide bonds into conserved framework regions (see

Reiter et al. Stabilization of the Fv fragments in recombinant immunotoxins by disulfide bonds engineered into conserved framework regions, Biochemistry 1994, 33, 6551-5459). Such scFv fragments are also known as disulfide-stabilized scFv fragments (ds-scFv).

In particular, in the context of the present invention, the binding moiety having a first binding site for a first antigen may be a divalent antibody fragment. The term “divalent” used herein, means that an antibody fragment is engineered by being linked to a second antibody fragment. For example, a divalent antibody fragment as disclosed herein may be a divalent Single-chain Fv fragment (scFv). A divalent (or bivalent) single-chain variable fragment (di-scFvs, bi-scFvs) can be engineered by linking two scFvs. This can be done by producing a single peptide chain with two VH and two VL regions, yielding tandem scFvs (Leber M F, Efferth T. Molecular principles of cancer invasion and metastasis (review). International journal of oncology 2009; 34.881-95; Hanahan D, Weinberg R A. Hallmarks of cancer: the next generation. Cell 2011; 144:646-74.), these formats can be composed from variable fragments with specificity for two different antigens, in which case they are types of bispecific antibodies (Wang M, Yin B, Wang H Y, Wang R-F. Current advances in T-cell-based cancer immunotherapy. Immunotherapy 2014; 6:1265-78; Lee D W, Gardner R, Porter D L, Louis C U, Ahmed N, Jensen M, et al. Current concepts in the diagnosis and management of cytokine release syndrome. Blood 2014; 124:188-95). The furthest developed of these are bispecific tandem di-scFvs, known as bi-specific T-cell engagers (BiTE antibody constructs, described elsewhere herein). Also, a divalent antibody fragment as disclosed herein may be a F(ab)2′-fragment. “F(ab′)2 fragment” is the proteolytic fragment of a pepsin-digested immunoglobulin. A “Fab¹ portion” is the product resulting from reducing the disulfide bonds of an F(ab′)2 portion. F(ab′)2 fragments have two antigen-binding F(ab) portions linked together by disulfide bonds, and therefore are divalent with a molecular weight of about 110 kDa.

In the context of the present invention, the binding moiety having a first binding site for a first antigen may alternatively be a monovalent antibody fragment. The term “monovalent antibody fragment” refers to an antibody fragment with affinity for one epitope, or antigen. A monovalent antibody fragment in the context of the present invention may be a binding moiety, an Fv fragment as defined elsewhere herein, a single chain Fv fragment as defined elsewhere herein (scFv), or a camelid single domain antibody. A “camelid single domain antibody” (sdAb), is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜˜25 kDa, two variable domains, one from a light and one from a heavy chain).

The recombinant proteinaceous binding molecule according to the present invention may alternatively comprise a binding moiety having a first binding site for a first antigen which is a binding molecule with antibody-like binding properties. With the term “antibody-like binding proteins” it is meant that the binding moiety is capable of binding an antigen in a manner similar to an antibody molecule. Examples of binding molecules with antibody-like (binding) properties that can be used as binding moiety having a first binding site for a first antigen include, but are not limited to, an aptamer (which are RNA or DNA moieties), or proteinaceous binding molecules such as an affilin, an affibody, an affimer, an atrimer, a mutein based on a polypeptide of the lipocalin family (also known as an anticalin@), an adnectin, an avimer, an alphabody, an autofluorescent protein, a centyrin, a DARPin, a fynomer, a glubody, a kappabody, a Kringle domain, a Kunitz domain, a knottin, a nanofitin, a repebody, an antigen specific t-cell receptor, any receptor-protein, or any other receptor-protein ligand. Such proteinaceous binding molecule having antibody like properties are well-know to the person skilled in the art and described, for example, in the review article of Skerra, A. (2001) Rev. Mol. Biotechnol. 74, 257-275 ‘Anticalins’: a new class of engineered ligand-binding proteins with antibody-like properties” or the review of Skerra (2000), “Engineered scaffolds for molecular recognition” J Mol Recognit, 13:167-187.

The term “antibody” generally refers to a proteinaceous binding molecule with immunoglobulin-like functions. Typical examples of an antibody are immunoglobulins, as well as derivatives or functional fragments thereof which still retain the binding specificity. Techniques for the production of antibodies are well known in the art. The term “antibody” also includes immunoglobulins (Ig's) of different classes (i.e. IgA, IgG, IgM, IgD and IgE) and subclasses (such as lgG1, lgG2 etc.). Illustrative examples of an antibody are Fab fragments, F(ab′)2 fragments, Fv fragments, single-chain Fv fragments (scFv), diabodies or domain antibodies (Holt L J et al., Trends Biotechnol. 21(11), 2003, 484-490). Domain antibodies may be single domain antibodies, single variable domain antibodies or immunoglobulin single variable domains. Such an immunoglobulin single variable domain may not only encompass an isolated antibody single variable domain polypeptide, but also a larger polypeptide that includes or consists of one or more monomers of an antibody single variable domain polypeptide sequence. The definition of the term “antibody” thus also includes embodiments such as chimeric, single chain and humanized antibodies.

A recombinant proteinaceous binding molecule according to the invention may carry one or more domains that have a sequence with at least about 60%, at least about 70%, at least about 75%, at least about 80%, at least about 85%, at least about 90%, at least about 92%, at least about 95%, at least about 96%, at least about 97%, at least about 98% or at least about 99% sequence identity with a corresponding naturally occurring domain of an immunoglobulin M, an immunoglobulin G, an immunoglobulin A, an immunoglobulin D

or an immunoglobulin E. It is noted in this regard, the term “about” or “approximately” as used herein means within a deviation of 20%, such as within a deviation of 10% or within 5% of a given value or range.

Accordingly, the main chain (longer polypeptide chain) of a recombinant proteinaceous binding molecule of the invention may include domains with the above sequence identity with a corresponding domain of an immunoglobulin mu heavy chain, of an immunoglobulin gamma heavy chain, of an immunoglobulin alpha heavy chain, of an immunoglobulin delta heavy chain or of an immunoglobulin epsilon heavy chain. Further, a recombinant proteinaceous binding molecule of the invention may include, including consist of, domains with the above sequence identity with a corresponding domain of an immunoglobulin lambda light chain or of an immunoglobulin kappa light chain. The entire heavy chain domains of a recombinant proteinaceous binding molecule according to the invention may have at least about 60%, at least about 70%, at least about 75%, at least about 80%, at least about 85%, at least about 90%, at least about 92%, at least about 95% at least about 97%, at least about 98% or at least about 99% sequence identity with the corresponding regions of an immunoglobulin mu heavy chain, of an immunoglobulin gamma heavy chain (such as gamma 1, gamma 2, gamma 3 or gamma 4 heavy chains), of an immunoglobulin alpha heavy chain (such as alpha 1 or alpha 2 heavy chains), of an immunoglobulin delta heavy chain or of an immunoglobulin epsilon heavy chain. The light chain domains present in a recombinant proteinaceous binding molecule according to the invention may have at least about 60%, at least about 70%, at least about 75%, at least about 80%, at least about 85%, at least about 90%, at least about 92%, at least about 95%, at least about 97%, at least about 98% or at least about 99% sequence identity with the corresponding regions of an immunoglobulin lambda light chain (such as lambda 1, lambda 2, lambda 3 or lambda 4 light chains) or of an immunoglobulin kappa light chain.

“Percent (%) sequence identity” with respect to amino acid sequences disclosed herein is defined as the percentage of amino acid residues in a candidate sequence that are pair-wise identical with the amino acid residues in a reference sequence, i.e. an recombinant proteinaceous binding molecule of the present disclosure, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared. The same is true for nucleotide sequences disclosed herein.

In accordance with the explanations given above, in some preferred embodiments, the binding moiety with antibody-like binding properties is a DARPin. DARPins are proteinaceous binding molecules with antibody-like binding properties typically exhibiting highly specific and high-affinity target protein binding. They are derived from natural ankyrin repeat proteins, one of the most common classes of binding proteins in nature, which are responsible for diverse functions such as cell signaling, regulation and structural integrity of the cell. DARPins consist of at least three repeat motifs or modules, of which the most island the most C-terminal modules are referred to as “caps”, since they shield the hydrophobic core of the protein. The number of internal modules is indicated as number (e.g. N1C, N2C, N3C) while the caps are indicated with “N” or “C”, respectively. The molecular mass of e.g.

14 or 18 kDa (kilodaltons) for four-(N2C) or five-(N3C) repeat DARPins is rather small for a biologic (ca 10% of the size of an IgG). DARPins constitute a new class of potent, specific and versatile small-protein therapeutics, and are used as investigational tools in various research, diagnostic and therapeutic applications (Pluckthun A (2015). “Designed ankyrin repeat proteins (DARPins): binding proteins for research, diagnostics, and therapy”. Annu. Rev. Pharmacol. Toxicol. 55 (1): 489-511).

In particular, the recombinant proteinaceous binding molecule according to the present invention is also referred to as “KiHss hemibody”, as defined elsewhere herein. In some embodiments, a recombinant proteinaceous binding molecule of the invention may comprise an antibody fragment (as a binding moiety) with specificity against one tumor associated target antigen and a VH or VL comprising one half of a binding site for CD3, defined elsewhere herein. In preferred embodiments, a KiHss hemibody may be VL anti Epcam, comprising a VL UCHT 1 fused to a CH3 hole chain (having the sequence shown in SEQ ID No.:2), and a binding moiety consisting of a Darpin antiEpcam Ec4 fused to a CH3 knob chain (having the sequence shown in SEQ ID No.:4). In further preferred embodiments, a KiHss hemibody may be VH anti Epcam, comprising a VH UCHT1 fused to a CH3 hole chain (having the sequence shown in SEQ ID No.: 1), and a binding moiety consisting of a Darpin antiEpcam Ec4 fused to a CH3 knob chain (having the sequence shown in SEQ ID No.:4). In preferred embodiments, a KiHss hemibody may be VL anti EGFR, comprising a VL UCHT1 fused to a CH3 hole chain (having the sequence shown in SEQ ID No.:2), and a binding moiety consisting of a Darpin antiRORI G3w fused to a CH3 knob chain (having the sequence shown in SEQ ID No.:51). In further preferred embodiments, a KiHss hemibody may be VH anti EGFR, comprising a VH UCHT1 fused to a CH3 hole chain (having the sequence shown in SEQ ID No.:1), and a binding moiety consisting of a Darpin antiRORI G3w fused to a CH3 knob chain (having the sequence shown in SEQ ID No.:51). As used herein VL or VH UCHT1 refers to a VH or a VL of a binding site for human CD3. In further preferred embodiments, a KiHss hemibody may be VL anti EGFR, comprising a VL UCHT1

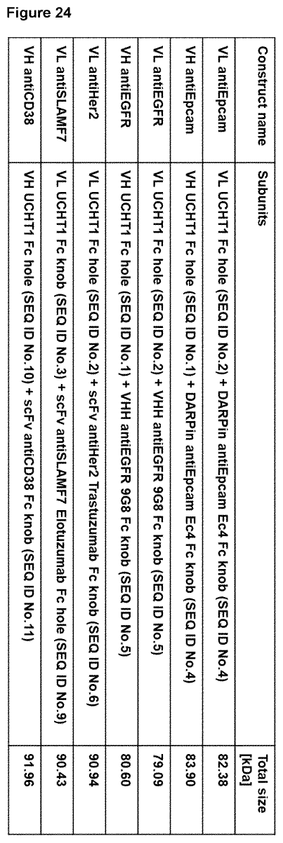

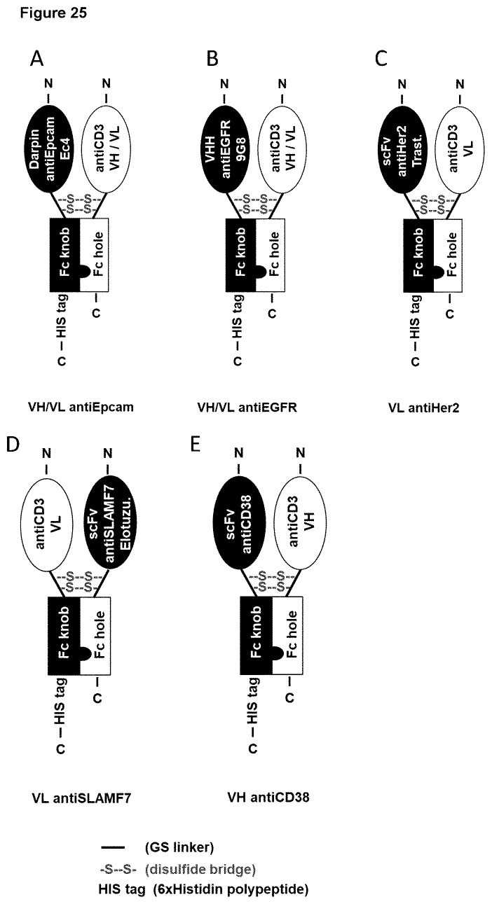

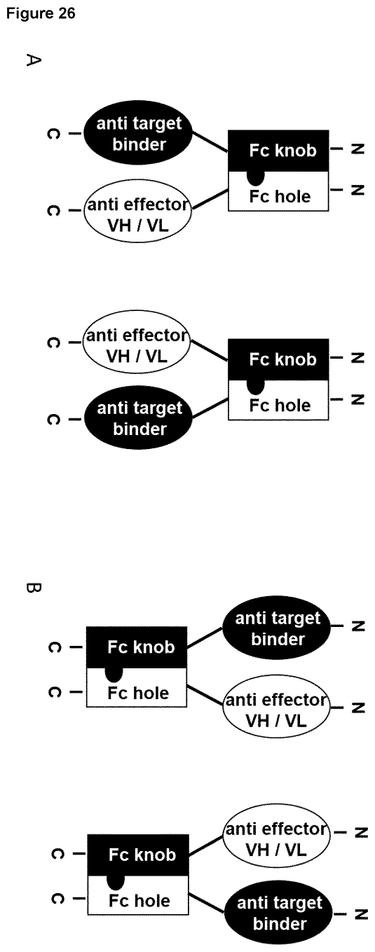

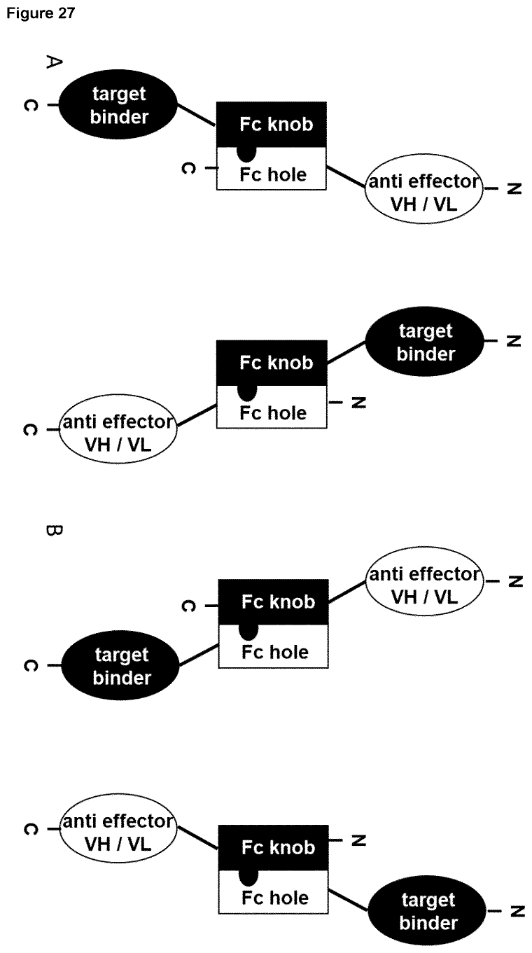

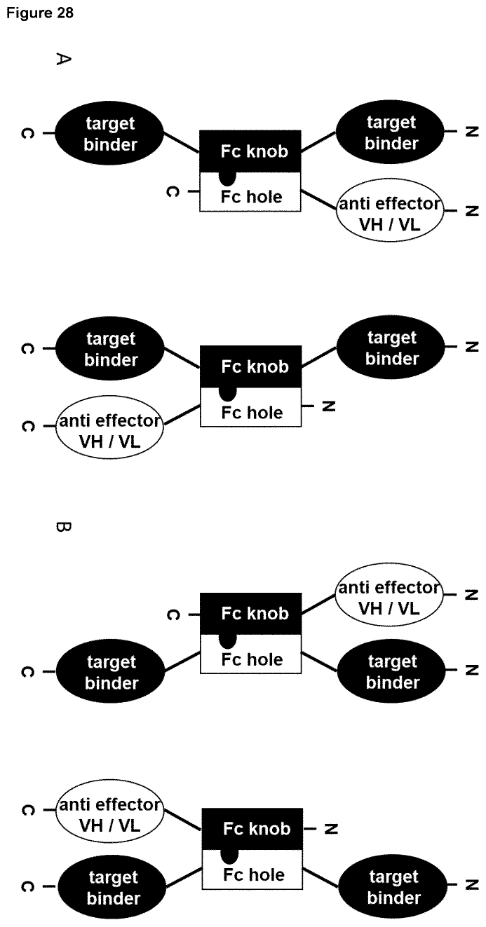

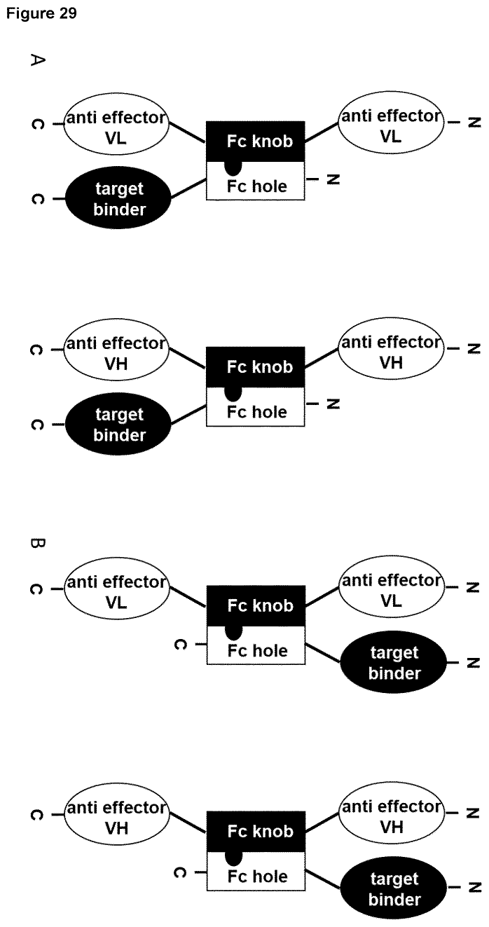

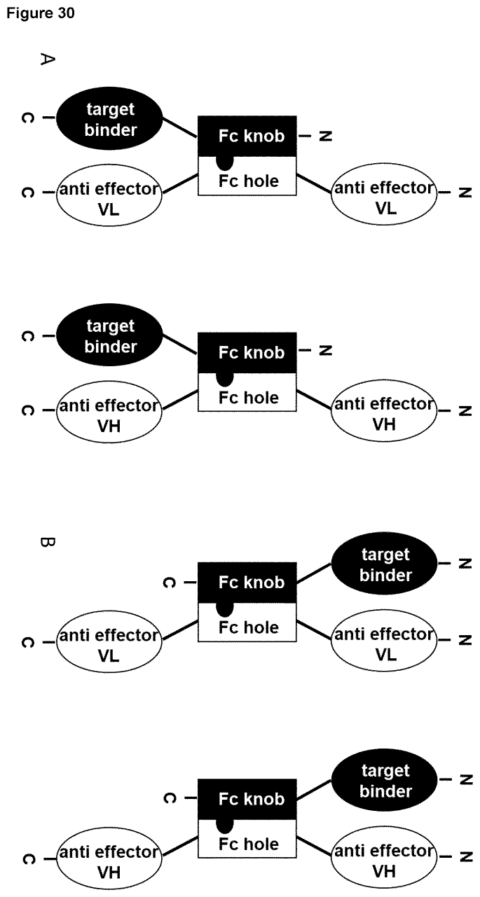

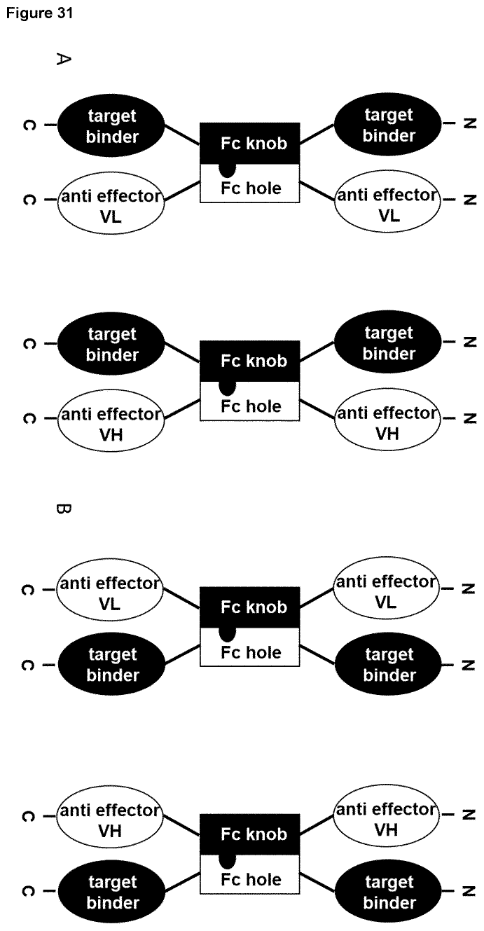

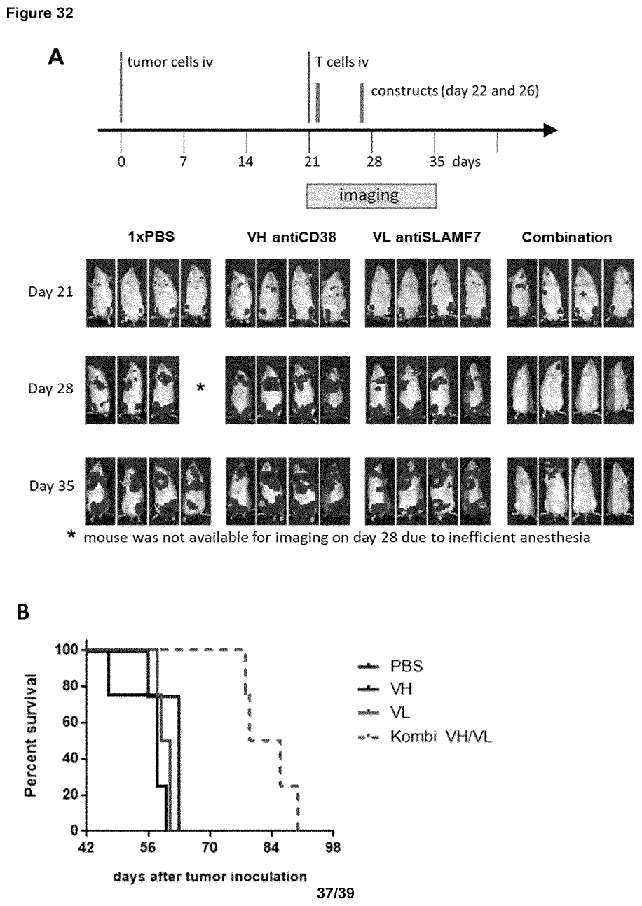

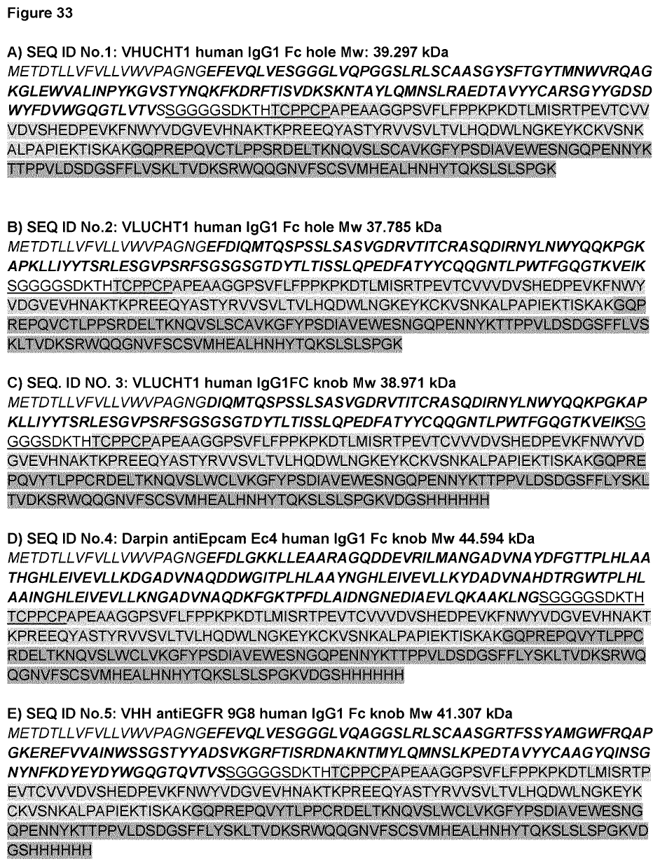

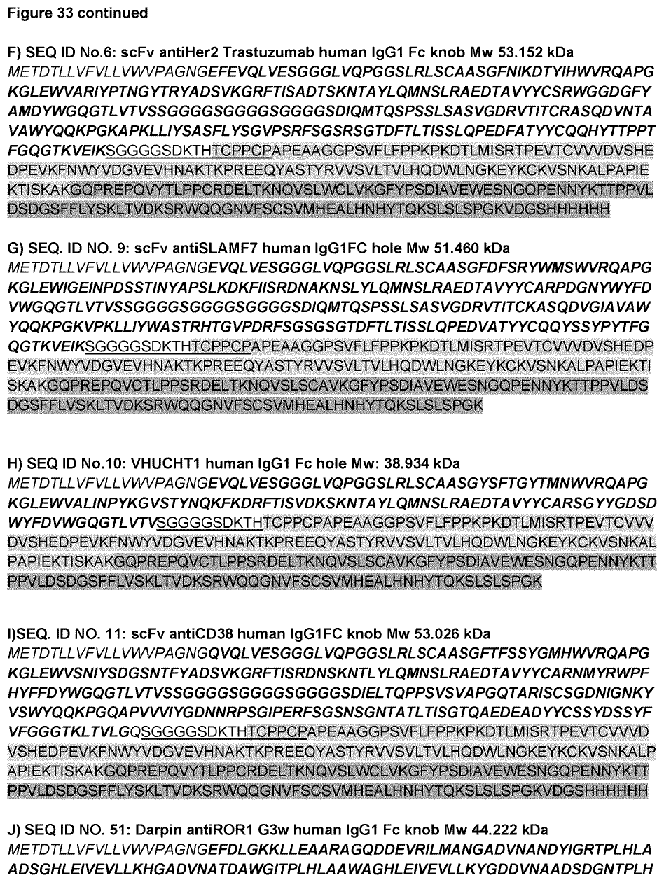

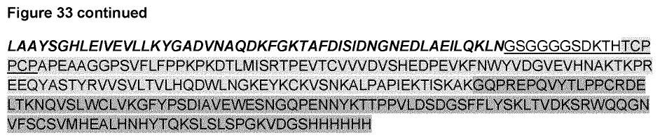

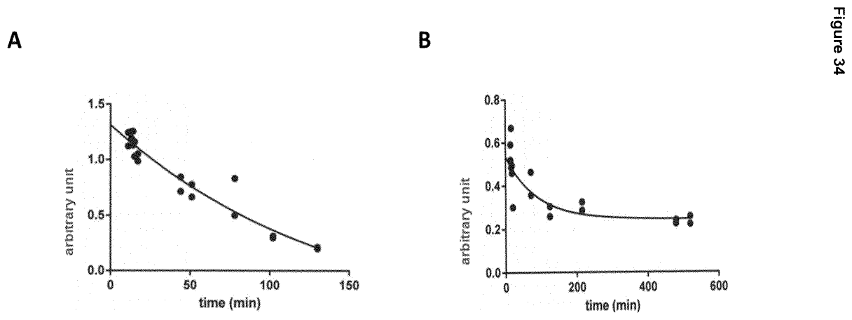

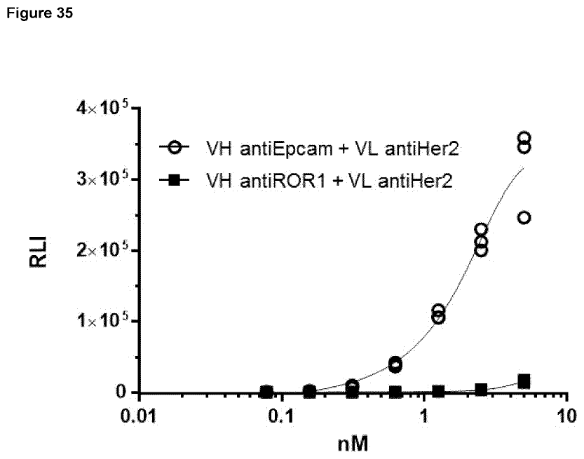

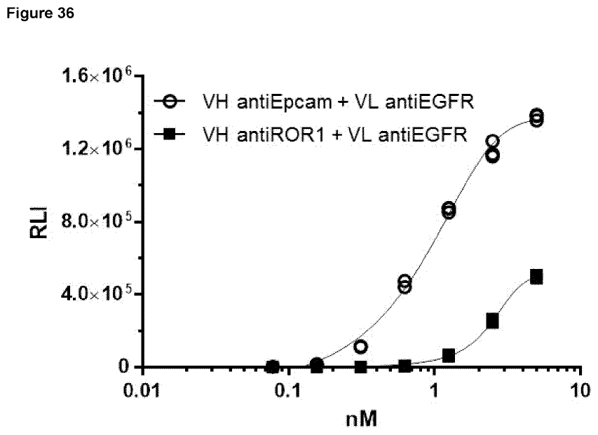

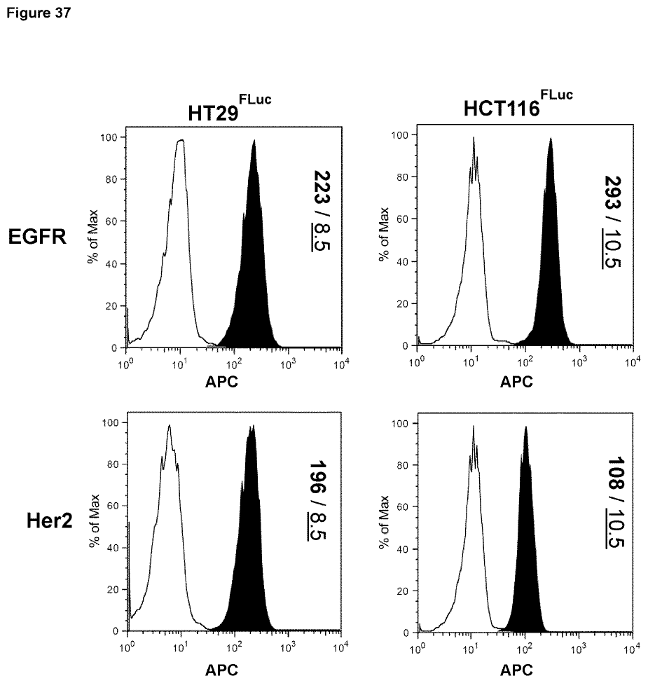

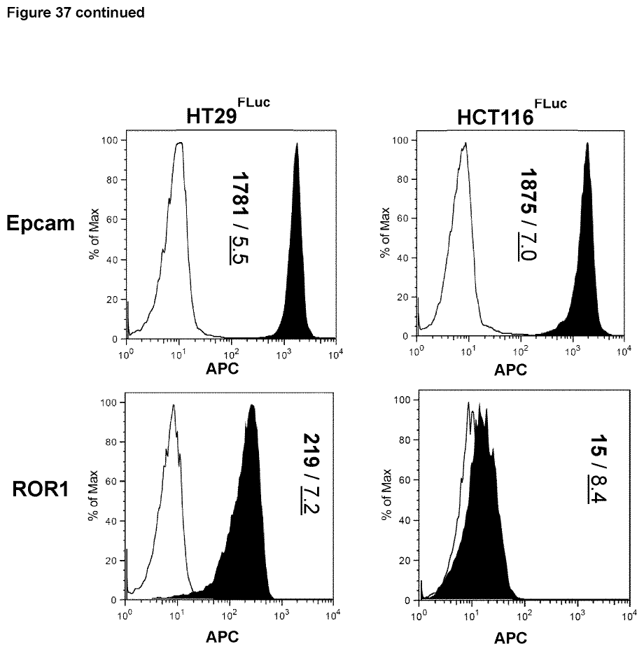

fused to a CH3 hole chain (having the sequence shown in SEQ ID No.:2), and a binding moiety consisting of a camelid single domain antibody VHH antiEGFR 9G8 fused to a CH3 knob chain (having the sequence shown in SEQ ID No.:5). In further preferred embodiments, a KiHss hemibody may be VH anti EGFR, comprising a VL UCHT1 fused to a CH3 hole chain (having the sequence shown in SEQ ID No.:2), and a binding moiety consisting of a camelid single domain antibody VHH antiEGFR 9G8 fused to a CH3 knob chain (having the sequence shown in SEQ ID No.:5). In further preferred embodiments, a KiHss hemibody may be VL anti Her2, comprising a VL UCHT1 fused to a CH3 hole chain (having the sequence shown in SEQ ID No.:2), and a binding moiety consisting of a scFv antiHerTrastuzumab fused to a CH3 knob chain (having the sequence shown in SEQ ID No.:6). In further preferred embodiments, a KiHss hemibody may be VL antiSLAMF7, comprising a VL UCHT1 fused to a CH3 knob chain (having the sequence shown in SEQ ID No.:5), and a binding moiety consisting of a scFv antiSLAMF7 fused to a CH3 hole chain (having the sequence shown in SEQ ID No.:9). In further preferred embodiments, a KiHss hemibody may be VH antiCD38, comprising a VH UCHT1 fused to a CH3 hole chain (having the sequence shown in SEQ ID No.:2), and a binding moiety consisting of a scFv antiCD38 fused to a CH3 knob chain (having the sequence shown in SEQ ID No.: 11). In further embodiments a KiHss hemibody may be VLdiL2k anti SLAMF7 comprising scFv antiSLAMF7 human IgGIFC hole (SEQ. ID NO: 9) and VLdiL2k human IgGIFC knob (SEQ ID NO: 31) (diL2K is the de-immunized version of the mouse monoclonal antibody L2K, Micromet/Amgen) The above mentioned constructs are also depicted in FIG. 24, FIG. 25 and FIG. 34.

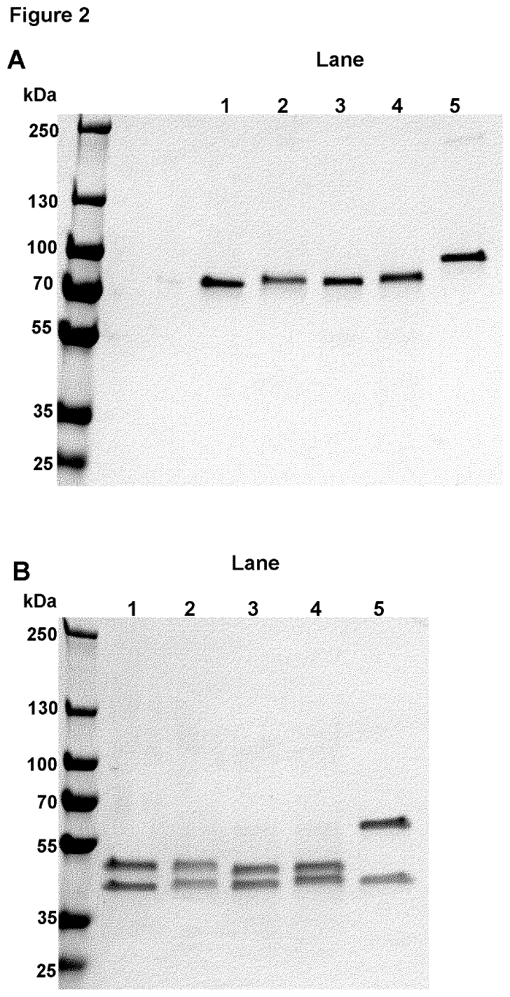

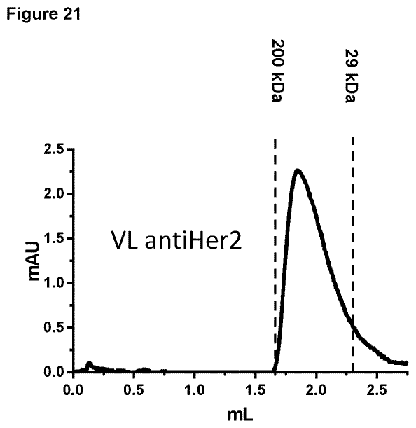

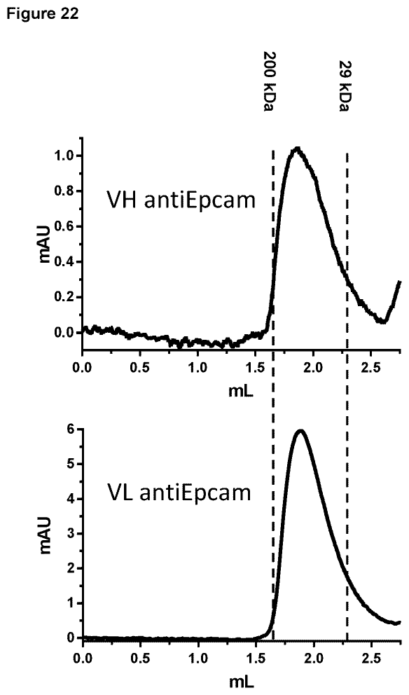

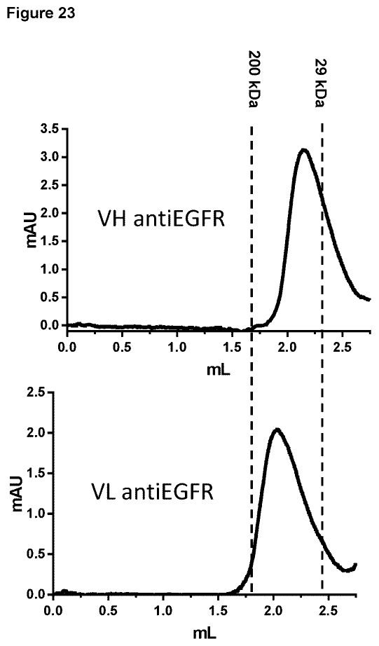

The electrophoretic separation of the purified KiHss hemibody Constructs is shown in FIG. 2. In FIG. 21 Size exclusion chromatography of Hemibody constructs VL antiHer2 is shown. FIG. 22 shows size exclusion chromatography of hemibody constructs VH anti Epcam and VL antiEpcam, and FIG. 23 shows size exclusion chromatography of hemibody constructs VH antiEGFR and VL antiEGFR. The sequence numbers and the size of said constructs are summarized in FIG. 24. FIG. 25 shows a scheme of the same KiHss hemibody constructs.

According to the invention, the recombinant proteinaceous binding molecule has and immunoglobulin CH3 domain of the first or the second heavy chain which comprises the amino acid substitution T366W. Said immunoglobulin CH3 domain is also referred to herein as “CH3 knob-chain” or “Fc knob-chain”. Further, the recombinant proteinaceous binding molecule has an immunoglobulin CH3 domain of the other heavy chain which comprises at least one of the amino acid substitutions T366S, L368A and Y407V. Said immunoglobulin CH3 domain is also referred to herein as “CH3 hole-chain” or “Fc hole chain”. As defined elsewhere herein, said complementary mutations were added to the CH3 domain of each Fc heavy chain to increase the probability of heterodimer formation.

Furthermore, to increase the stability further, two cysteine mutations were added (CH3 knob chain: S354C, CH3 hole chain: Y349C) enabling the formation of a disulfide bridge.

The recombinant proteinaceous binding molecule of the invention may have at least one amino acid residue of the CH2 domain that is able to mediate binding to Fc receptors which is lacking or mutated. In particular, the recombinant proteinaceous binding molecule of the invention may have the amino acid residues selected from the group consisting of sequence position 230, 231, 232, 233, 234, 235, 236, 237, 238, 265, 297, 327, and 330 (numbering of sequence positions according to the EU-index, wherein the least one mutation is preferably selected from the group consisting of a substitution Leu234-la, a substitution Leu235-la, and a substitution Asn297-Ala and a substitution Pro329-la, more preferably selected from the group consisting of a substitution Leu234-la, a substitution Leu235-la, and a substitution Asn297-la as also illustrated, e.g., throughout the appended Examples. These mutations may be introduced in order to avoid Fc-mediated target cell killing through a single recombinant proteinaceous binding protein (also referred to herein as KiHss hemibodies), which means before the formation of heterodimer resulting from the association of two recombinant proteinaceous binding molecules—i.e formation of heterodimers of recombinant proteinaceous binding molecules (monomers) as defined elsewhere herein, via the sulfide stabilized knob-into-hole technology characterized by the complementary mutations in the CH3 domain of each Fc heavy chain of each monomer—(A. Margaret Merchant, Zhenping Zhu, Jean Q. Yuan, Audrey Goddard, Camellia W. Adams. An efficient route to human bispecific IgG. Nature Biotechnology 1998:677-81). In particular, the recombinant proteinaceous binding molecule may comprise mutations that lead to effector silencing and aglycosylation of the Fc fragment, thereby reducing the interaction with Fc receptors FcyRs and C1q. Said mutations ensure that only specific scFv mediated tumor cell killing and no ADCC or CDC occurs. In this context, the recombinant proteinaceous binding molecule may comprise mutations in the CH2 domain which lead to effector silencing and aglycosylation and reduce the interaction with FcyRs and C1q. In preferred embodiments, said mutations may be mutations located in the CH2 region. Preferably said mutations are selected from the group consisting of a substitution Leu234-la, a substitution Leu235-la, a substitution Asn297-la and a substitution Pro329-la. In another preferred embodiments, the recombinant proteinaceous binding molecule may comprise four mutations in the CH2 domain of the Fc fragment consisting of a substitution Leu234-la, a substitution Leu235-Ala, a substitution Asn297-la and a substitution Pro329-Ala. More preferably, as e.g. illustrated throughout the appended Examples, the recombinant proteinaceous binding molecule may comprise three mutations in the CH2 domain of the Fc

fragment consisting of a substitution Leu234-la, a substitution Leu235-la and a substitution Asn297-Ala.

In some embodiments, an antigen to which a recombinant proteinaceous binding molecule according to the invention binds is an antigen that is included in the extracellular matrix or it is a cell surface antigen. In some embodiments an antigen to which a recombinant proteinaceous binding molecule according to the invention binds is a tumor associated antigen. In some embodiments, the first binding site of a first binding moiety binds a tumor associated antigen. In other embodiments, the tumor associated antigen is located on the vasculature of a tumor. Illustrative examples of a tumor associated antigen include, but are not limited to SlamF7, CD10, CD19, CD20, CD21, CD22, CD25, CD30, CD33, CD34, CD37, CD38, CD44v6, CD45, CDw52, CD70, CD117, CD123, CD133, CD135, CD138, CD140a, CD140b, CD171, CD309 CSPG4, Muc-1,Muc-16 Erb-B1, Erb-B2, Erb-B3, EGFRvlll, Folate receptor, PSMA, PSCA, PSA, VEGFR2, TAG-72, HLA-DR, IGFR, IL3R, fibroblast activating protein (FAP), CEA, EpCAM, Claudin6, CLL-1, EphAIO, G250, BB2, gp100, NY-ESO-1, LAGE-1, MAGE-A1, MAGE-A3, P-Cadherin, N-Cadherin, E-Cadherin, HLA-DP, HLA-A2, CCR4, CXCR3, FGFR1, GPC3, GPA33, GD2, BCMA, ROR1, TIM-3, and Mesothelin (MSLN).

It is understood that such a tumour associated antigen may be a cell surface antigen or be included in the extracellular matrix. In preferred embodiments, the tumor associated antigen is a cell surface antigen.

The term “extracellular matrix” refers to the tissue region of a multicellular animal, including a human that is found in the intercellular space, i.e. between the cells of the respective tissue. The extracellular matrix is largely a network of proteins such as fibrillar and non-fibrillar collagens or elastin, of glycoproteins such as laminin or fibronectin, of proteoglycans, such as chondroitin sulfate or keratan sulphate and of polysaccharides such as Hyaluronic acid. The extracellular matrix serves inter alia in segregating different tissues from each other or in regulating intercellular communication. In some embodiments a tumor associated antigen may be expressed partly or exclusively at the extracellular matrix of a tumor.

The term “cell surface antigen” as used herein refers to a molecule that is displayed on the surface of a cell. Typically, such a molecule is located in or on the plasma membrane of the cell such that at least part of this molecule remains accessible from the ambience, i.e. from outside the cell. A respective molecule consists of or includes typically amino acid and/or saccharide moieties. An illustrative example of a cell surface molecule, which is located in the plasma membrane, is a transmembrane protein that, in its three-dimensional conformation, has regions of hydrophilicity and hydrophobicity. One or more hydrophobic region(s) allow(s) the cell surface molecule to be embedded or inserted in the hydrophobic plasma membrane of the cell whereas hydrophilic regions of the protein extend on either side of the plasma membrane into the cytoplasm and extracellular space, respectively. Examples of a cell surface molecule located on the plasma membrane include, but are not limited to, a protein with a posttranslationally modified cysteine residue carrying a palmitoyl group, a protein modified at a C-terminal cysteine residue carrying a farnesyl group or a protein modified at the C-terminus carrying a glycosyl phosphatidyl inositol (“GPI”) anchor. These groups allow covalent attachment of proteins to the outer surface of the plasma membrane, where they remain accessible for recognition by extracellular molecules such as antibodies. Examples of cell surface antigens include a cell surface receptor molecule such as a G protein coupled receptor (e.g. the b-adrenergic receptor), a tyrosin kinase receptor (such as EGFR, EGFRvlll, Her2/neu, HER2/c-neu, PDGFRa, ILR-1, TNFR, CD30, CD33 or GMCSFR), a membrane receptor with associated tyrosin kinase activity (such as IL6R or LIFR) or a membrane receptor with Ser/Thr kinase activity (such as TGF R), to name only a few examples.

Examples of a tumor associated antigen that is included in the extracellular matrix include, but are not limited to, a proteoglycan such as Melanoma-associated Chondroitin Sulfate Proteoglycan (CSPG4) or CD44v6, including a mucin such as Muc-1 or a membrane-bound enzyme such as Carbonic anhydrase IX (CAIX). Additional examples for such antigens are tenascin and the fibroblast activating protein (FAP).

In the context of the present invention, the recombinant proteinaceous binding molecule may have a second binding site for a second antigen which binds a T-cell, NK (natural killer), Monocyte, Macrophage or Neutrophilic Granulocyte cell specific receptor molecule (CD32a, CD89, CD64, NKp30, NKp40, PD1, CTLA4, LFA1). In particular, the T-cell- or NK cell specific receptor molecule may be one of CD3, the T cell receptor (TCR), CD28, CD16, NKG2D, 0x40, 4-1 BB, CD2, CD4, CD5, CD8, CD95, CD32a, CD64, CD89, NKp30, NKp40, PD1 CTLA4, CD40 or LFA1. In this context, the TCR is TCR (alpha/beta), TCR (gamma/delta), or the CD3 variant gamma/epsilon or the CD3 variant delta/epsilon.

The recombinant proteinaceous binding molecule of the invention may have an architecture as defined herein: namely a binding moiety, a variable domain of either the light chain or the heavy chain, and an Fc fragment. In particular, the binding moiety may be fused to the C- or N-terminus of the “Fc knob” or of the “Fc hole” chain, and the variable domain of either the light chain or the heavy chain may be fused to the C- or N-terminus of the “Fc knob” or of the “Fc hole” chain, as depicted in FIGS. 25 to 27. The recombinant proteinaceous binding molecule may also have more than one binding moiety and more than one variable domain. For example, the recombinant proteinaceous binding protein may have two binding moieties, fused at the C- or N-terminus of the “Fc knob-chain” or the “Fc hole-chain” of the Fc fragment and one variable domain of either the light chain or the heavy

chain, fused at the C- or N-terminus of the “Fc knob-chain” or the “Fc hole-chain” of the Fc fragment. The two binding moieties present on one KiHss hemibody may each have a first binding site capable of binding an antigen, wherein said antigen is of the same identity. Therefore, the two binding moieties have specificity for the same antigen, as defined elsewhere herein. This type of recombinant proteinaceous binding molecule is depicted for example in FIG. 28. The recombinant proteinaceous binding molecule may have one binding moiety, fused to the C- or N-terminus of the “Fc knob” or of the “Fc hole” chain of the Fc fragment and two variable domains of either the light chain or the heavy chain, fused to the C- or N-terminus of the of the “Fc knob” or of the “Fc hole” chain of the Fc fragment, The two variable domain of either the light chain or the heavy chain may have one half of a binding site for the same antigen, Therefore, the two VH/VL may have specificity for the same antigen, as defined elsewhere herein. This type of recombinant proteinaceous binding molecule is depicted in FIG. 29 and FIG. 30. The recombinant proteinaceous binding molecule may also have two binding moieties, fused at the C- or N-terminus of the “Fc knob chain” or the “Fc hole chain” of the Fc fragment and two variable domains of either the light chain or the heavy chain, fused at the C- or N-terminus of the “Fc knob chain” or the “Fc hole chain” of the Fc fragment. The two binding moieties may have specificity for the same antigen and the two variable domains of either the heavy or the light chain may have specificity for the same antigen. This type of recombinant proteinaceous binding molecule is depicted for example in FIG. 31.

The present invention also relates to a heterodimeric recombinant proteinaceous binding molecule comprising a heterodimer of the recombinant proteinaceous molecules described elsewhere herein, which constitute the monomers of the heterodimer. In particular, a heterodimeric recombinant proteinaceous binding molecule comprises a heterodimer of the recombinant proteinaceous binding molecule (also referred to as KiHss hemibody) of the present invention. Therefore, the first monomer consists of a binding moiety having a first binding site for a first antigen; a variable domain of either an antibody light chain (VL) or an antibody heavy chain (VH) of a second binding site for a second antigen and an an Fc fragment. The VL/VH have therefore one half of a second binding site for a second antigen, (therefore being an incomplete binding site which, as disclosed elsewhere herein, will form a complete second binding site by association to a single VH or VL domain of a second monomer). The Fc fragment comprises a first and a second heavy chain, wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable.

Moreover, the variable domain of either the antibody light chain or the antibody heavy chain and the first binding moiety are linked via the Fc fragment.

The second monomer of the heterodimer consists of a binding moiety having a first binding site for a first antigen; a variable domain of either an antibody light chain (VL) or an antibody heavy chain (VH) of a second binding site for a second antigen and an Fc fragment. The VL/VH have therefore one half of a second binding site for a second antigen, (therefore being an incomplete binding site which, as disclosed elsewhere herein, will form a complete second binding site by association to a single VH or VL domain of a second monomer). The Fc fragment comprises a first and a second heavy chain, wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable. Also in the second monomer, the variable domain of either the antibody light chain or the antibody heavy chain and the first binding moiety are linked via the Fc fragment. In the heterodimer disclosed herein above, the first antigen of the binding moiety of the first monomer and the first antigen of the binding moiety of the second monomer may be two antigens of the same identity. Therefore, the first binding site of the binding moiety of the first monomer and the first binding site of the binding moiety of the second monomer have binding specificity for the same tumor associated antigens. However, in preferred embodiments, as disclosed herein, the first antigen of the binding moiety of the first monomer and the first antigen of the binding moiety of the second monomer may be two antigens of different identity.

As outlined elsewhere herein, the second binding site for a second antigen is formed by the single VH and VL domains present in two different monomers. For the association of these two single domains it is necessary that they come into close contact. This is the case upon binding to the specific epitope(s) they recognise. Thus, the association of the two monomers takes place on the target cell, defined elsewhere herein, comprising the epitope(s) to be detected. To ensure optimal association of the recombinant proteinaceous binding molecules of the invention, preferably these single domains should be obtained from only one antibody such as the UCHT-1 antibody as described herein. However, it can also be possible to combine single VH and VL domains within the KiHss-formate from different antibodies. For example, such VH/VL domains could be obtained from different antibodies, which epitopes are located spatially close to each other or which have similar or overlapping epitopes, or generated by phage display techniques. Thus, dimerization of the monomers into a heterodimer is mediated by the association of the single (unpaired) VH and VL domains of the two KiHss hemibodies. For dimerization to occur a spatial adjacency is necessary. This adjacency is primarily achieved by the binding to the targeted epitope(s).

According to the invention the heterodimeric recombinant proteinaceous binding molecule may comprise monomers (i. e two recombinant proteinaceous binding molecules, or KiHss hemibodies) wherein the first antigen of the binding moiety of the first monomer and the first antigen of the binding moiety of the second monomer are two antigens of different identity. Therefore, the first binding site of the binding moiety of the first monomer and the first binding site of the binding moiety of the second monomer have binding specificity for two different tumor associated antigens. Accordingly, the present invention also relates to a heterodimeric recombinant proteinaceous binding molecule comprising a heterodimer of a recombinant proteinaceous molecules (monomers), wherein the first monomer consists of a binding moiety having a first binding site for a first antigen; a variable domain of either an antibody light chain or an antibody heavy chain of a second binding site for a second antigen; and an Fc fragment as defined elsewhere herein. In particular, the first antigen of the first monomer and the first antigen of the second monomer are two antigens of different identity. The Fc fragment of said heterodimer may comprise a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and wherein the variable domain of either the antibody light chain or the antibody heavy chain and the first binding moiety are linked via the Fc fragment.

The second monomer of the heterodimer consists of a binding moiety comprising a first binding site for a first antigen, a variable domain of either an antibody light chain (VL) or an antibody heavy chain (VH) of a second binding site for a second antigen and an an Fc fragment. The VL/VH have therefore one half of a second binding site for a second antigen, (therefore being an incomplete binding site which, as disclosed elsewhere herein, will form a complete second binding site by association to a single VH or VL domain of a second monomer). The Fc fragment comprises a first and a second heavy chain, wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable. Also in the second monomer, the variable domain of either the antibody light chain or the antibody heavy chain and the first binding moiety are linked via the Fc fragment, wherein the variable domain of an antibody light chain of the second binding site for a second antigen of the first monomer and the variable domain of an antibody heavy chain of the second binding site for a second antigen of the second monomer associate thereby forming the second binding site and dimerizing the heterodimer.

The heterodimeric recombinant proteinaceous binding molecule of the invention may therefore comprise a binding moiety which comprises a first binding site which is an antibody fragment, as defined elsewhere herein. Said antibody fragment may be selected from the group consisting of a divalent antibody fragment, and a monovalent antibody fragment, both defined elsewhere herein. Furthermore, said divalent antibody fragment may be an (Fab)2′-fragment, or a divalent single-chain Fv fragment, defined elsewhere herein. Said monovalent antibody fragment may be selected from the group consisting of a binding moiety, a Fv fragment, a single-chain Fv fragment (scFv) and a camelid single domain antibody, as defined elsewhere herein. The heterodimeric recombinant proteinaceous binding molecule of the invention may have a binding moiety having a first binding site for a first antigen which is a binding molecule with antibody-like binding properties, defined elsewhere herein. Such a binding molecule with antibody-like binding properties may, for example, by an aptamer, i.e. an oligonucleotide (DNA or RNA molecule) or a peptide molecule that bind to a specific target molecule. Alternatively, a binding molecule with antibody-like binding properties may also be a proteinaceous binding molecule with antibody-like binding properties. Examples of proteinaceous binding molecule with antibody-like binding properties include, but are not limited to, an affilin, an affibody, an affimer, an atrimer, an anticalin, an adnectin, an avimer, an alphabody, an autofluorescent protein, a centyrin, a DARPin, a fynomer, a glubody, a kappabody, a Kringle domain, a Kunitz domain, a knottin, a nanofitin, a repebody, an antigen specific t-cell receptor, any receptor-protein, or any receptor-protein ligand.

The possible architecture of the monomers forming the heterodimeric recombinant proteinaceous binding molecule of the invention is as defined elsewhere herein and are shown in FIGS. 25 to 31

According to the invention, the monomers of the heterodimeric recombinant proteinaceous binding molecule of the invention have an immunoglobulin CH3 domain of the first or the second heavy chain which comprises the amino acid substitution T366W. Said immunoglobulin CH3 domain is also referred to herein as “CH3 knob-chain” or “Fc knob-chain”. Furthermore, the monomers of the heterodimeric recombinant proteinaceous binding molecule have an immunoglobulin CH3 domain of the other heavy chain which comprises at least one of the amino acid substitutions T366S, L368A and Y407V. Said immunoglobulin CH3 domain is also referred to herein as “CH3 hole-chain” or “Fc hole chain”. Further to further increase the stability, two cysteine mutations were added (CH3 knob chain: S354C, CH3 hole chain: Y349C) enabling the formation of a disulfide bridge, as described elsewhere herein.

Furthermore, the monomers of the heterodimeric recombinant proteinaceous binding molecule of the invention have at least one amino acid residue of the CH2 domain that is able to mediate binding to Fc receptors which is lacking or mutated, as defined elsewhere herein. In particular, said lacking or mutated amino acid residues may be selected from the group consisting of sequence position 230, 231, 232, 233, 234, 235, 236, 237, 238, 265, 297, 327, and 330 (numbering of sequence positions according to the (EU-index), wherein the least one mutation is preferably selected from the group consisting of a substitution Leu234->Ala, a substitution Leu235->Ala, and a substitution Asn297->Ala and a substitution Pro329->Ala. These mutations may be introduced in order to avoid Fc-mediated target cell killing through a single recombinant proteinaceous binding protein (also referred to herein as KiHss hemibodies), which means before the formation of heterodimer resulting from the association of two recombinant proteinaceous binding molecules—i.e formation of heterodimers of recombinant proteinaceous binding molecules (monomers) as defined elsewhere herein, via the sulfide stabilized knob-into-hole technology characterized by the complementary mutations in the CH3 domain of each Fc heavy chain of each monomer. In particular, the monomers of the recombinant proteinaceous binding molecule may comprise four additional mutations in the CH2 region, preferably said mutations are a substitution Leu234->Ala, a substitution Leu235->Ala, a substitution Asn297->Ala and a substitution Pro329->Ala. These mutations led to effector silencing and aglycosylation respectively and reduced the antibody interaction with FcyRs and C1q. This ensured that only specific scFv mediated tumor cell killing and no ADCC or CDC occurs.

As defined elsewhere herein, an antigen to which a recombinant proteinaceous binding molecule, or a monomer of the heterodimeric recombinant proteinaceous binding molecule of the invention may bind, is an antigen that is included in the extracellular matrix or it is a cell surface antigen. In some embodiments an antigen to which a recombinant proteinaceous binding molecule according to the invention binds is a tumor associated antigen. In some embodiments, the first binding site of the first monomer and/or the third binding site of the second monomer binds a tumor associated antigen. In other embodiments, the tumor associated antigen is located on the vasculature of a tumor. In further embodiments, the tumor associated antigen is selected from the group consisting of SlamF7, CD10, CD19, CD20, CD21, CD22, CD25, CD30, CD33, CD34, CD37, CD38, CD44v6, CD45, CDw52, CD70, CD 117, CD123, CD133, CD135, CD138, CD140a, CD140b, CD171, CD309 CSPG4, Muc-1,Muc-16 Erb-B1, Erb-B2, Erb-B3, EGFRvIll, Folate receptor, PSMA, PSCA, PSA, VEGFR2, TAG-72, HLA-DR, IGFR, IL3R, fibroblast activating protein (FAP), CEA, EpCAM, Claudin6, CLL-1, EphA10, G250, BB2, gp100, NY-ESO-1, LAGE-1, MAGE-A1, MAGE-A3, P-Cadherin, N-Cadherin, E-Cadherin, HLA-DP, HLA-A2, CCR4, CXCR3, FGFR1, GPC3, GPA33, GD2, BCMA, ROR1, TIM-3, and Mesothelin (MSLN).

Regarding the monomers according to the invention, the second binding site may be a binding site which binds a T-cell, NK (natural killer), Monocyte, Macrophage, Dendritic cell, or Neutrophilic Granulocyte cell specific receptor molecule, such as a receptor molecule selected from the group consisting of CD32a, CD89, CD64, NKp30, NKp40, PD1, CTLA4, LFA1. In particular, the T-cell- or NK cell specific receptor molecule may be one of CD3, the T cell receptor (TCR), CD28, CD16, NKG2D, 0x40, 4-1 BB, CD2, CD4, CD5, CD8, CD95, CD32a, CD64, CD89, NKp30, NKp40, PD1 CTLA4, CD40 or LFA1. In this context, the TCR is TCR (alpha/beta), TCR (gamma/delta), or the CD3 vaiant gamma/epsilon or the CD3 variant delta/epsilon.

The invention also provides a pharmaceutical composition that includes a recombinant proteinaceous binding molecule of the invention and, optionally a pharmaceutically acceptable excipient.

The recombinant proteinaceous binding molecule according to the invention can be administered via any parenteral or non-parenteral (enteral) route that is therapeutically effective for proteinaceous drugs. Parenteral application methods include, for example, intracutaneous, subcutaneous, intramuscular, intratracheal, intranasal, intravitreal or intravenous injection and infusion techniques, e.g. in the form of injection solutions, infusion solutions or tinctures, as well as aerosol installation and inhalation, e.g. in the form of aerosol mixtures, sprays or powders. An overview about pulmonary drug delivery, i.e. either via inhalation of aerosols (which can also be used in intranasal administration) or intracheal instillation is given by J. S. Patton et al. The lungs as a portal of entry for systemic drug delivery. Proc. Amer. Thoracic Soc. 2004 Vol. 1 pages 338-344, for example). Non-parenteral delivery modes are, for instance, orally, e.g. in the form of pills, tablets, capsules, solutions or suspensions, or rectally, e.g. in the form of suppositories recombinant proteinaceous binding molecule of the invention can be administered systemically or topically in formulations containing conventional non-toxic pharmaceutically acceptable excipients or carriers, additives and vehicles as desired.

When the pharmaceutical is administered parenterally to a mammal, and in particular to humans corresponding administration methods may include, but are not limited to, for example, intracutaneous, subcutaneous, intramuscular, intratracheal or intravenous injection and infusion techniques, e.g. in the form of injection solutions, infusion solutions or tinctures as well as aerosol installation and inhalation, e.g. in the form of aerosol mixtures, sprays or powders. A combination of intravenous and subcutaneous infusion and/or injection might be most convenient in case of compounds with a relatively short serum half life. The pharmaceutical composition may be an aqueous solution, an oil-in water emulsion or a water-in-oil emulsion.

In this regard it is noted that transdermal delivery technologies, e.g. iontophoresis, sonophoresis or microneedle-enhanced delivery, as described in Meidan V M and Michniak BB 2004 Am. J. Ther. 11(4): 312-316, can also be used for transdermal delivery of a recombinant proteinaceous binding molecule described herein. Non-parenteral delivery modes are, for instance, oral, e.g. in the form of pills, tablets, capsules, solutions or suspensions, or rectal administration, e.g. in the form of suppositories. The recombinant proteinaceous binding molecule of the invention can be administered systemically or topically in formulations containing a variety of conventional non-toxic pharmaceutically acceptable excipients or carriers, additives, and vehicles.

The dosage of the recombinant proteinaceous binding molecule applied may vary within wide limits to achieve the desired preventive effect or therapeutic response. It will, for instance, depend on the affinity of the recombinant proteinaceous binding molecule for a chosen target as well as on the half-life of the complex between the antibody molecule and the ligand in vivo. Further, the optimal dosage will depend on the biodistribution of the recombinant proteinaceous binding molecule or a conjugate thereof, the mode of administration, the severity of the disease/disorder being treated as well as the medical condition of the patient. For example, when used in an ointment for topical applications, a high concentration of the recombinant proteinaceous binding molecule can be used. However, if wanted, the recombinant proteinaceous binding molecule may also be given in a sustained release formulation, for example liposomal dispersions or hydrogel-based polymer microspheres, like PolyActive™ or OctoDEX™ (cf. Bos et al., Business Briefing: Pharmatech 2003: 1-6). Other sustained release formulations available are for example PLGA based polymers (PR pharmaceuticals), PLA-PEG based hydrogels (Medincell) and PEA based polymers (Medivas). Accordingly, the recombinant proteinaceous binding molecule of the present invention can be formulated into compositions using pharmaceutically acceptable ingredients as well as established methods of preparation (Gennaro, A. L. and Gennaro, A. R. (2000) Remington: The Science and Practice of Pharmacy, 20th Ed., Lippincott Williams & Wlkins, Philadelphia, PA). To prepare the pharmaceutical compositions, pharmaceutically inert inorganic or organic excipients can be used. To prepare e.g. pills, powders, gelatine capsules or suppositories, for example, lactose, talc, stearic acid and its salts, fats, waxes, solid or liquid polyols, natural and hardened oils can be used. Suitable excipients for the production of solutions, suspensions, emulsions, aerosol mixtures or powders for reconstitution into solutions or aerosol mixtures prior to use include water, alcohols, glycerol, polyols, and suitable mixtures thereof as well as vegetable oils.

The pharmaceutical composition may also contain additives, such as, for example, fillers, binders, wetting agents, glidants, stabilizers, preservatives, emulsifiers, and

furthermore solvents or solubilizers or agents for achieving a depot effect. The latter is that fusion proteins may be incorporated into slow or sustained release or targeted delivery systems, such as liposomes and microcapsules.

The formulations can be sterilized by numerous means, including filtration through a bacteria-retaining filter, or by incorporating sterilizing agents in the form of sterile solid compositions which can be dissolved or dispersed in sterile water or other sterile medium just prior to use.

Numerous possible applications for the inventive recombinant proteinaceous binding molecule exist in medicine. In addition to their use in in vitro diagnostics or drug delivery, a recombinant proteinaceous binding molecule of the invention, which binds, for example, tissue- or tumor-specific cellular surface molecules can be generated.

As explained elsewhere herein, a recombinant proteinaceous binding molecule according to the invention may be directed against any desired target epitopes/antigens. Depending on the selected epitopes/antigens, the recombinant proteinaceous binding molecule may be suitable in the treatment or prevention of disease. Accordingly, a recombinant proteinaceous binding molecule according to the invention may be used in a method of treating and/or preventing a medical condition such as a disorder or disease. Similarly, the recombinant proteinaceous binding molecules of the present invention as well as the heterodimeric recombinant proteinaceous binding molecules can be used in the treatment of a disease. Where the recombinant proteinaceous binding molecule is capable of activating immune cells in an FcR-dependent manner, it may be particularly useful to select a recombinant proteinaceous binding molecule that has an Fc-corresponding portion that shows reduced binding to Fc-receptors. By this means an undesired immune activation mediated by FcR binding is prevented. A disease to be treated or prevented may be a proliferatory disease. Examples of a proliferative disease include, but are not limited to, hemopoietic malignancies, such as acute and chronic myeloic and lymphatic leukemias, as well as lymphomas, or solid tumors. Examples of solid tumors include, but are not limited to, tumors of the gastrointestinal tract such as colon, bone, lung, kidney, prostate, breast, brain, ovary, uterus, testis, mesenchymal tumors and skin, such as melanoma. FIG. 32 of the present invention shows KiHss Hemibodies successfully targeting the multiple myeloma associated antigens CD38 and SLAMF7 in vivo As can be seen in FIG. 32B higher survival rate was observed in mice treated with the combination of two KiHSS hemibody, forming in vivo an heterodimer on a target cell by association of their respective VH and VL, as described elsewhere herein. As such the term “treat”, “treating” or “treatment” as used herein means to reduce (slow down, lessen), stabilize or inhibit or at least partially alleviate or abrogate the progression of the symptoms associated with the respective disease. Thus, it includes the administration of said recombinant proteinaceous binding molecule, preferably in the form of a medicament, to a subject. The term “prevent”, “preventing”, “prevention” as used herein refers to prophylactic or preventative measures, wherein the subject is to prevent an abnormal, including pathologic, condition in the organism which would then lead to the defined disease. Thus, it also includes the administration of said recombinant proteinaceous binding molecule, preferably in the form of a medicament, to a subject, defined elsewhere herein. Those in need of the prevention include those prone to having the disease, defined elsewhere herein. In other words, those who are of a risk to develop such disease and will thus probably suffer from said disease in the near future.

The term “subject” when used herein includes mammalian and non-mammalian subjects. Preferably the subject of the present invention is a mammal, including human. In some embodiment the mammal is a mouse. A subject also includes human and veterinary patients. Where the subject is a living human who may receive treatment for a disease or condition as described herein, it is also addressed as a “patient”. Those in need of treatment include those already suffering from the disease. Preferably, a treatment reduces (slows down, lessens), stabilizes, or inhibits or at least partially alleviates or abrogates progression of a symptom that is associated with the presence and/or progression of a disease or pathological condition. “Treat”, “treating”, or “treatment” refers to a therapeutic treatment.

Turning now to nucleic acids of the invention, a nucleic acid molecule encoding a binding moiety, a VH or VL, and/or an Fc fragment of a recombinant proteinaceous binding molecule according to the invention may be any nucleic acid in any possible configuration, such as single stranded, double stranded or a combination thereof. Nucleic acids include for instance DNA molecules, RNA molecules, analogues of the DNA or RNA generated using nucleotide analogues or using nucleic acid chemistry, locked nucleic acid molecules (LNA), and protein nucleic acids molecules (PNA). DNA or RNA may be of genomic or synthetic origin and may be single or double stranded. Such nucleic acid can be e.g. mRNA, cRNA, synthetic RNA, genomic DNA, cDNA synthetic DNA, a copolymer of DNA and RNA, oligonucleotides, etc. A respective nucleic acid may furthermore contain non-natural nucleotide analogues and/or be linked to an affinity tag or a label.

A nucleic acid sequence encoding a binding moiety, a VH or VL, and an Fc fragment of a recombinant proteinaceous binding molecule according to the invention is included in a vector such as a plasmid. Where a substitution or deletion is to be included, for example, in an Fc fragment, when compared to a naturally occurring immunoglobulin domain of an Fc fragment, the coding sequence of the respective native domain/region, e.g. included in the sequence of an immunoglobulin, can be used as a starting point for the mutagenesis. For the mutagenesis of selected amino acid positions, the person skilled in the art has at his disposal the various established standard methods for site-directed mutagenesis. A commonly used technique is the introduction of mutations by means of PCR (polymerase chain reaction)

using mixtures of synthetic oligonucleotides, which bear a degenerate base composition at the desired sequence positions. For example, use of the codon NNK or NNS (wherein N=adenine, guanine or cytosine or thymine; K=guanine or thymine; S=adenine or cytosine) allows incorporation of all 20 amino acids plus the amber stop codon during mutagenesis, whereas the codon VVS limits the number of possibly incorporated amino acids to 12, since it excludes the amino acids Cys, lie, Leu, Met, Phe, Trp, Tyr, Val from being incorporated into the selected position of the polypeptide sequence; use of the codon NMS (wherein M=adenine or cytosine), for example, restricts the number of possible amino acids to 11 at a selected sequence position since it excludes the amino acids Arg, Cys, Gly, lie, Leu, Met, Phe, Trp, Val from being incorporated at a selected sequence position. In this respect it is noted that codons for other amino acids (than the regular 20 naturally occurring amino acids) such as selenocystein or pyrrolysine can also be incorporated into a nucleic acid of a recombinant proteinaceous binding molecule molecule. It is also possible, as described by Wang, L, et al. (2001) Science 292, 498-500, or Wang, L, and Schultz, P. G. (2002) Chem. Comm. 1, 1-11, to use “artificial” codons such as UAG which are usually recognized as stop codons in order to insert other unusual amino acids, for example o-methyl-L-tyrosine or p-aminophenylalanine.

The use of nucleotide building blocks with reduced base pair specificity, as for example inosine, 8-oxo-2′deoxyguanosine or 6(2-deoxy-D-ribofuranosyl)-3,4-dihydro-8H-pyrimin-do-1,2-oxazine-7-one, is another option for the introduction of mutations into a chosen sequence segment. A further possibility is the so-called triplet-mutagenesis. This method uses mixtures of different nucleotide triplets, each of which codes for one amino acid, for incorporation into the coding.

A nucleic acid molecule encoding a binding moiety, a VH or VL, and an Fc fragment of a recombinant proteinaceous binding molecule according to the invention can be expressed using any suitable expression system, for example in a suitable host cell or in a cell-free system. The obtained recombinant proteinaceous binding molecule is enriched by means of selection and/or isolation. Thus, in one embodiment, the nucleic acid molecule of the present invention can be comprised in a vector. Similarly, the nucleic acid molecule of the present invention may be comprised in a host cell or the vector comprising the nucleic acid molecule of the present invention may be comprised in a host cell (Stadler C R, Bahr-Mahmud H, Celik L, et al Elimination of large tumors in mice by mRNA-encoded bispecific antibodies. Nat Med. 2017 July; 23(7):815-81). Thus, by the approach as described by Stadler et al, it is possible to recombinantly express recombinant proteinaceous binding molecule of the invention directly in a patient.

Methods of making recombinant proteinaceous binding molecule of the invention are known in the art, e.g. chemical conjugation. Alternatively, recombinant proteinaceous binding molecules disclosed herein may be produced recombinantly. A recombinant proteinaceous binding molecule of the invention may be produced using any known and well-established expression system and recombinant cell culturing technology, for example, by expression in bacterial hosts (prokaryotic systems), or eukaryotic systems such as yeasts, fungi, insect cells or mammalian cells. A recombinant proteinaceous binding molecule of the present invention may be produced in transgenic organisms such as a goat, a plant or a XENOMOUSE transgenic mouse, an engineered mouse strain that has large fragments of the human immunoglobulin loci and is deficient in mouse antibody production. A recombinant proteinaceous binding molecule may also be produced by chemical synthesis.

For recombinant production of a recombinant proteinaceous binding molecule of the invention typically a polynucleotide encoding the recombinant proteinaceous binding molecule is isolated and inserted into a replicable vector such as a plasmid for further cloning (amplification) or expression. An illustrative example of a suitable expression system is a glutamate synthetase system (such as sold by Lonza Biologies), with the host cell being for instance CHO, HEK293 or NSO. A polynucleotide encoding the recombinant proteinaceous binding molecule is readily isolated and sequenced using conventional procedures. Vectors that may be used include plasmid, virus, phage, transposons, minichromsomes of which plasmids are a typical embodiment. Generally, such vectors further include a signal sequence, origin of replication, one or more marker genes, an enhancer element, a promoter and transcription termination sequences operably linked to the light and/or heavy chain polynucleotide so as to facilitate expression. Polynucleotides encoding the light and heavy chains may be inserted into separate vectors and transfected into the same host cell or, if desired both the heavy chain and light chain can be inserted into the same vector for transfection into the host cell. Both chains can, for example, be arranged, under the control of a dicistronic operon and expressed to result in the functional and correctly folded antibody molecule as described in Skerra, A. (1994) Use of the tetracycline promoter for the tightly regulated production of a murine antibody fragment in Escherichia coli, Gene 151, 131-135, or Skerra, A. (1994) A general vector, pASK84, for cloning, bacterial production, and single-step purification of antibody Fab fragments, Gene 141, 79-8. Thus, the present invention also relates to a process of constructing a vector encoding the recombinant proteinaceous binding molecule of the invention, which method includes inserting into a vector, a polynucleotide encoding the binding moiety, the VH or VL and the CH3-hole and CH3-knob chain of a recombinant proteinaceous binding molecule of the invention.

When using recombinant techniques, the recombinant proteinaceous binding molecule can be produced intracellularly, in the periplasmic space, or directly secreted into the medium. If the recombinant proteinaceous binding molecule is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E coli. The recombinant proteinaceous binding molecule can also be produced in any oxidizing environment. Such an oxidizing environment may be provided by the periplasm of Gram-negative bacteria such as E. coli, in the extracellular milieu of Gram-positive bacteria or in the lumen of the endoplasmatic reticulum of eukaryotic cells (including animal cells such as insect or mammalian cells) and usually favors the formation of structural disulfide bonds. It is, however, also possible to produce a recombinant proteinaceous binding molecule of the invention in the cytosol of a host cell such as E. coli. In this case, the polypeptide can either be directly obtained in a soluble and folded state or recovered in form of inclusion bodies, followed by renaturation in vitro. A further option is the use of specific host strains having an oxidizing intracellular milieu, which may thus allow the formation of disulfide bonds in the cytosol (Venturi M, Seifert C, Hunte C. (2002) “High level production of functional antibody Fab fragments in an oxidizing bacterial cytoplasm.” J. Mol. Biol. 315, 1-8).

The recombinant proteinaceous binding molecule produced by the cells can be purified using any conventional purification technology, for example, hydroxylapatite chromatography, gel electrophoresis, dialysis, and affinity chromatography, with affinity chromatography being one preferred purification technique recombinant proteinaceous binding molecules may be purified via affinity purification with proteins/ligands that specifically and reversibly bind constant domains such as the CH1 or the CL domains. Examples of such proteins are immunoglobulin-binding bacterial proteins such as Protein A, Protein G, Protein A/G or Protein L, wherein Protein L binding is restricted to recombinant proteinaceous binding molecules that contain kappa light chains. An alternative method for purification of antibodies with kappa-light chains is the use of bead coupled anti kappa antibodies (KappaSelect). The suitability of protein A as an affinity ligand depends on the species and isotype of any immunoglobulin Fc fragment that is present in the recombinant proteinaceous binding molecule. Protein A can be used to purify recombinant proteinaceous binding molecule (Lindmark et al., 1983 Binding of immunoglobulins to protein A and immunoglobulin levels in mammalian sera J. Immunol. Meth. 62: 1-13). Protein G is recommended for all mouse isotypes and for human gamma3 (Guss et al. 1986, Structure of the IgG-binding regions of streptococcal protein G EMBO J. 5: 15671575). The choice of the purification method that is used for a particular recombinant proteinaceous binding molecule of the invention is within the knowledge of the person of average skill in the art.

It is also possible to equip one of the chains of the recombinant proteinaceous binding molecule of the invention with an affinity tag. Affinity tags such as the Strep-tag® or Strep-tag® II (Schmidt, T. G. M. et al. (1996) J. Mol. Biol. 255, 753-766), the myc-tag, the

FLAG™-tag, the His6-tag or the HA-tag allow easy detection and also simple purification of the recombinant proteinaceous binding molecule.

Thus, a method of producing are combinant proteinaceous binding molecule of the present invention comprises expressing a nucleic acid encoding the recombinant proteinaceous binding molecule under conditions allowing expression of the nucleic acid, preferably the recombinant proteinaceous binding molecule is expressed in a host cell or a cell-free system.

It is possible to insert the coding sequences encoding for recombinant proteinaceous binding molecules such as the binding moiety, a VH or VL, and an Fc fragment as defined elsewhere herein, into one expression vector. Thus, a method of producing a recombinant proteinaceous binding molecule comprises expressing a nucleic acid encoding the recombinant proteinaceous binding molecule under conditions allowing expression of the nucleic acid, preferably the recombinant proteinaceous binding molecule is expressed in a host cell or a cell-free system. Informations on the design, expression, isolation and target antigen binding of the recombinant proteinaceous binding molecule of the invention are summarized in Examples 1 and 5.

The present invention further relates to a use of a recombinant proteinaceous binding molecule of the present invention for the treatment of a disease, wherein the recombinant proteinaceous binding molecule forms a heterodimer only in vivo on a target cell, thereby reducing “off target activation”. “Off target activation” could be any activation of cells, which is not due to the cells to be targeted by the used recombinant proteinaceous binding molecules. For example, an off target activation could be a target cell independent T cell activation, which even may become exaggerated in the presence of endothelial cells. Also encompassed is the so-called cytokine storm. This is an immune reaction consisting of a positive feedback loop between cytokines and immune cells, with highly elevated levels of various cytokines. Thus, in preferred embodiments, the recombinant proteinaceous binding molecule provides for target cell restricted T cell-activation. The disease to be treated may be a proliferatory disease.

The following sequences summarized in Table 1 have been referred to by in the present disclosure.

TABLE 1

| SEQ. | | |
|---|---|---|
| ID | | |
| NO. | Name | Sequence |
| 1 | VHUCHT1 | METDTLLVFVLLVWVPAGNGEFEVQLVESG |
| | human IgG1 Fc | GGLVQPGGSLRLSCAASGYSFTGYTMNWV |
| | hole MW 39.297 | RQAPGKGLEWVALINPYKGVSTYNQKFKDR |
| | kDa | FTISVDKSKNTAYLQMNSLRAEDTAVYYCAR |
| | | SGYYGDSDWYFDVWGQGTLVTVSSGGGGS |
| | | DKTHTCPPCPAPEAAGGPSVFLFPPKPKDTL |
| | | MISRTPEVTCVVVDVSHEDPEVKFNWYVDG |
| | | VEVHNAKTKPREEQYASTYRVVSVLTVLHQD |
| | | WLNGKEYKCKVSNKALPAPIEKTISKAKGQPR |
| | | EPQVCTLPPSRDELTKNQVSLSCAVKGFYPSD |
| | | IAVEWESNGQPENNYKTTPPVLDSDGSFFLVS |
| | | KLTVDKSRWQQGNVFSCSVMHEALHNHYTQK |
| | | SLSLSPGK |
| 2 | VLUCHT1 | METDTLLVFVLLVWVPAGNGEFDIQMTQSPSS |
| | human IgG1 Fc | LSASVGDRVTITCRASQDIRNYLNWYQQKPGK |
| | hole | APKLLIYYTSRLESGVPSRFSGSGSGTDYTLTIS |
| | | SLQPEDFATYYCQQGNTLPWTFGQGTKVEIKS |
| | | GGGGSDKTHTCPPCPAPEAAGGPSVFLFPPKP |
| | | KDTLMISRTPEVTCVVVDVSHEDPEVKFNWYVD |
| | | GVEVHNAKTKPREEQYASTYRVVSVLTVLHQDW |
| | | LNGKEYKCKVSNKALPAPIEKTISKAKGQPREPQ |
| | | VCTLPPSRDELTKNQVSLSCAVKGFYPSDIAVEW |
| | | ESNGQPENNYKTTPPVLDSDGSFFLVSKLTVDKS |
| | | RWQQGNVFSCSVMHEALHNHYTQKSLSLSPGK |
| 3 | VLUCHT1 | METDTLLVFVLLVWVPAGNGDIQMTQSPSSLSASV |
| | human IgG1Fc | GDRVTITCRASQDIRNYLNWYQQKPGKAPKLLIYYT |
| | knob | SRLESGVPSRFSGSGSGTDYTLTISSLQPEDFATYY |
| | | CQQGNTLPWTFGQGTKVEIKSGGGGSDKTHTCPP |
| | | CPAPEAAGGPSVFLFPPKPKDTLMISRTPEVTCVVV |
| | | DVSHEDPEVKFNWYVDGVEVHNAKTKPREEQYAS |
| | | TYRVVSVLTVLHQDWLNGKEYKCKVSNKALPAPIEK |
| | | TISKAKGQPREPQVYTLPPCRDELTKNQVSLWCLVK |
| | | GFYPSDIAVEWESNGQPENNYKTTPPVLDSDGSFFL |
| | | YSKLTVDKSRWQQGNVFSCSVMHEALHNHYTQKSL |
| | | SLSPGKVDGSHHHHHH |
| 4 | Darpin | METDTLLVFVLLVWVPAGNGEFDLGKKLLEAARA |
| | antiEpcam Ec4 | GQDDEVRILMANGADVNAYDFGTTPLHLAATHGH |
| | human IgG1 Fc | LEIVEVLLKDGADVNAQDDWGITPLHLAAYNGHLE |
| | knob | IVEVLLKYDADVNAHDTRGWTPLHLAAINGHLEIVE |
| | | VLLKNGADVNAQDKFGKTPFDLAIDNGNEDIAEVL |
| | | QKAAKLNGSGGGGSDKTHTCPPCPAPEAAGGPS |
| | | VFLFPPKPKDTLMISRTPEVTCVVVDVSHEDPEVK |
| | | FNWYVDGVEVHNAKTKPREEQYASTYRVVSVLTV |
| | | LHQDWLNGKEYKCKVSNKALPAPIEKTISKAKGQP |
| | | REPQVYTLPPCRDELTKNQVSLWCLVKGFYPSDIA |
| | | VEWESNGQPENNYKTTPPVLDSDGSFFLYSKLTVD |
| | | KSRWQQGNVFSCSVMHEALHNHYTQKSLSLSPGK |
| | | VDGSHHHHHH |
| 5 | VHH antiEGFR | METDTLLVFVLLVWVPAGNGEFEVQLVESGGGLVQ |
| | 9G8 human lgG1 | AGGSLRLSCAASGRTFSSYAMGWFRQAPGKEREF |
| | Fc knob | VVAINWSSGSTYYADSVKGRFTISRDNAKNTMYLQM |
| | | NSLKPEDTAVYYCAAGYQINSGNYNFKDYEYDYWG |
| | | QGTQVTVSSGGGGSDKTHTCPPCPAPEAAGGPSVF |
| | | LFPPKPKDTLMISRTPEVTCVVVDVSHEDPEVKFNW |
| | | YVDGVEVHNAKTKPREEQYASTYRVVSVLTVLHQD |
| | | WLNGKEYKCKVSNKALPAPIEKTISKAKGQPREPQV |
| | | YTLPPCRDELTKNQVSLWCLVKGFYPSDIAVEWESN |
| | | GQPENNYKTTPPVLDSDGSFFLYSKLTVDKSRWQQ |
| | | GNVFSCSVMHEALHNHYTQKSLSLSPGKVDGSHHH |
| | | HHH |
| 6 | scFv antiHer2 | METDTLLVFVLLVWVPAGNGEFEVQLVESGGGLVQ |
| | Trastuzumab | PGGSLRLSCAASGFNIKDTYIHWVRQAPGKGLEWV |
| | human IgG1 Fc | ARIYPTNGYTRYADSVKGRFTISADTSKNTAYLQMNSL |
| | knob | RAEDTAVYYCSRWGGDGFYAMDYWGQGTLVTVSSG |
| | | GGGSGGGGSGGGGSDIQMTQSPSSLSASVGDRVTI |
| | | TCRASQDVNTAVAWYQQKPGKAPKLLIYSASFLYSG |
| | | VPSRFSGSRSGTDFTLTISSLQPEDFATYYCQQHYTT |
| | | PPTFGQGTKVEIKSGGGGSDKTHTCPPCPAPEAAGG |
| | | PSVFLFPPKPKDTLMISRTPEVTCVVVDVSHEDPEVK |
| | | FNWYVDGVEVHNAKTKPREEQYASTYRVVSVLTVLH |
| | | QDWLNGKEYKCKVSNKALPAPIEKTISKAKGQPREPQ |
| | | VYTLPPCRDELTKNQVSLWCLVKGFYPSDIAVEWESN |
| | | GQPENNYKTTPPVLDSDGSFFLYSKLTVDKSRWQQG |
| | | NVFSCSVMHEALHNHYTQKSLSLSPGKVDGSHHHHHH |
| 7 | human IgG1 Fc | SGGGGSDKTHTCPPCPAPEAAGGPSVFLFPPKPKDT |
| | hole | LMISRTPEVTCVVVDVSHEDPEVKFNWYVDGVEVHN |
| | | AKTKPREEQYASTYRVVSVLTVLHQDWLNGKEYKCK |
| | | VSNKALPAPIEKTISKAKGQPREPQVCTLPPSRDELTK |
| | | NQVSLSCAVKGFYPSDIAVEWESNGQPENNYKTTPPV |
| | | LDSDGSFFLVSKLTVDKSRWQQGNVFSCSVMHEALHN |
| | | HYTQKSLSLSPGK |
| 8 | human IgG1 Fc | SGGGGSDKTHTCPPCPAPEAAGGPSVFLFPPKPKD |
| | knob | TLMISRTPEVTCVVVDVSHEDPEVKFNWYVDGVEVH |
| | | NAKTKPREEQYASTYRVVSVLTVLHQDWLNGKEYK |
| | | CKVSNKALPAPIEKTISKAKGQPREPQVYTLPPCRDEL |
| | | TKNQVSLWCLVKGFYPSDIAVEWESNGQPENNYKTT |
| | | PPVLDSDGSFFLYSKLTVDKSRWQQGNVFSCSVMHE |
| | | ALHNHYTQKSLSLSPGKVDGSHHHHHH |
| 9 | scFv | METDTLLVFVLLVWVPAGNGEVQLVESGGG |
| | antiSLAMF7 | LVQPGGSLRLSCAASGFDFSRYWMSWVRQA |
| | human IgG1Fc | PGKGLEWIGEINPDSSTINYAPSLKDKFIISRDN |
| | hole | AKNSLYLQMNSLRAEDTAVYYCARPDGNYWY |
| | | FDVWGQGTLVTVSSGGGGSGGGGSGGGGSD |
| | | IQMTQSPSSLSASVGDRVTITCKASQDVGIAVAW |
| | | YQQKPGKVPKLLIYWASTRHTGVPDRFSGSGSG |
| | | TDFTLTISSLQPEDVATYYCQQYSSYPYTFGQGT |
| | | KVEIKSGGGGSDKTHTCPPCPAPEAAGGPSVFL |
| | | FPPKPKDTLMISRTPEVTCVVVDVSHEDPEVKFN |
| | | WYVDGVEVHNAKTKPREEQYASTYRVVSVLTVL |
| | | HQDWLNGKEYKCKVSNKALPAPIEKTISKAKGQP |
| | | REPQVCTLPPSRDELTKNQVSLSCAVKGFYPSDIA |
| | | VEWESNGQPENNYKTTPPVLDSDGSFFLVSKLTV |
| | | DKSRWQQGNVFSCSVMHEALHNHYTQKSLSLSP |
| | | GK |
| 10 | VHUCHT1 | METDTLLVFVLLVWVPAGNGEVQLVESGGGLV |
| | human IgG1Fc | QPGGSLRLSCAASGYSFTGYTMNWVRQAPGK |
| | hole | GLEWVALINPYKGVSTYNQKFKDRFTISVDKSK |
| | | NTAYLQMNSLRAEDTAVYYCARSGYYGDSDW |
| | | YFDVWGQGTLVTVSGGGGSDKTHTCPPCPAP |
| | | EAAGGPSVFLFPPKPKDTLMISRTPEVTCVVVD |
| | | VSHEDPEVKFNWYVDGVEVHNAKTKPREEQYA |
| | | STYRVVSVLTVLHQDWLNGKEYKCKVSNKALPA |
| | | PIEKTISKAKGQPREPQVCTLPPSRDELTKNQVS |
| | | LSCAVKGFYPSDIAVEWESNGQPENNYKTTPPV |
| | | LDSDGSFFLVSKLTVDKSRWQQGNVFSCSVMH |
| | | EALHNHYTQKSLSLSPGK |
| 11 | scFv antiCD38 | METDTLLVFVLLVWVPAGNGQVQLVESGGG |
| | human IgG1Fc | LVQPGGSLRLSCAASGFTFSSYGMHWVRQA |
| | knob | PGKGLEWVSNIYSDGSNTFYADSVKGRFTIS |
| | | RDNSKNTLYLQMNSLRAEDTAVYYCARNMY |
| | | RWPFHYFFDYWGQGTLVTVSSGGGGSGGG |
| | | GSGGGGSDIELTQPPSVSVAPGQTARISCSG |
| | | DNIGNKYVSWYQQKPGQAPVVVIYGDNNRPS |
| | | GIPERFSGSNSGNTATLTISGTQAEDEADYYC |
| | | SSYDSSYFVFGGGTKLTVLGQSGGGGSDKT |
| | | HTCPPCPAPEAAGGPSVFLFPPKPKDTLMIS |
| | | RTPEVTCVVVDVSHEDPEVKFNWYVDGVEV |
| | | HNAKTKPREEQYASTYRVVSVLTVLHQDWL |
| | | NGKEYKCKVSNKALPAPIEKTISKAKGQPRE |
| | | PQVYTLPPCRDELTKNQVSLWCLVKGFYPS |
| | | DIAVEWESNGQPENNYKTTPPVLDSDGSFFL |
| | | YSKLTVDKSRWQQGNVFSCSVMHEALHNHY |
| | | TQKSLSLSPGKVDGSHHHHHH |
| 30 | VLdiL2k human | METDTLLVFVLLVWVPAGNGDIVLTQSPATLSL |
| | IgG1FC knob | SPGERATLSCRASQSVSYMNWYQQKPGKAPK |
| | | RWIYDTSKVASGVPARFSGSGSGTDYSLTINSL |
| | | EAEDAATYYCQQWSSNPLTFGGGTKVEIKGSG |
| | | GGGSDKTHTCPPCPAPEAAGGPSVFLFPPKPK |
| | | DTLMISRTPEVTCVVVDVSHEDPEVKFNWYVDG |
| | | VEVHNAKTKPREEQYASTYRVVSVLTVLHQDWL |
| | | NGKEYKCKVSNKALPAPIEKTISKAKGQPREPQV |
| | | YTLPPCRDELTKNQVSLWCLVKGFYPSDIAVEWE |
| | | SNGQPENNYKTTPPVLDSDGSFFLYSKLTVDKSR |
| | | WQQGNVFSCSVMHEALHNHYTQKSLSLSPGKVD |
| | | GSHHHHHH |
| 51 | Darpin antiROR1 | METDTLLVFVLLVWVPAGNGEFDLGKKLLEAARAGQDDEVRIL |
| | G3w human | MANGADVNANDYIGRTPLHLAADSGHLEIVEVLLKHGADVNAT |
| | IgG1 Fc knob | DAWGITPLHLAAWAGHLEIVEVLLKYGDDVNAADSDGNTPLHL |
| | | AAYSGHLEIVEVLLKYGADVNAQDKFGKTAFDISIDNGNEDLAEI |
| | | LQKLNGSGGGGSDKTHTCPPCPAPEAAGGPSVFLFPPKPKDT |
| | | LMISRTPEVTCVWVDVSHEDPEVKFNWYVDGVEVHNAKTKPRE |
| | | EQYASTYRVVSVLTVLHQDWLNGKEYKCKVSNKALPAPIEKTIS |
| | | KAKGQPREPQVYTLPPCRDELTKNQVSLWCLVKGFYPSDIAVE |
| | | WESNGQPENNYKTTPPVLDSDGSFFLYSKLTVDKSRWQQGNV |
| | | FSCSVMHEALHNHYTQKSLSLSPGKVDGSHHHHHH |

The present invention is further characterized by the following items:

1. A recombinant proteinaceous binding molecule comprising:
a) a first binding moiety, capable of binding an antigen, having a first binding site for a first antigen,
b) a variable domain of either an antibody light chain or an antibody heavy chain of a second binding site for a second antigen, and
c) an Fc fragment comprising a first and a second heavy chain,
wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, which interface comprises an original interface between the CH3 domains,
wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and
wherein the variable domain of either the antibody light chain or the antibody heavy chain and the first binding moiety are linked via the Fc fragment.

2. The recombinant proteinaceous binding molecule of item 1, wherein the binding moiety having a first binding site for a first antigen is an antibody fragment.

3. The recombinant proteinaceous binding molecule of item 2, wherein the antibody fragment is selected from the group consisting of a divalent antibody fragment, and a monovalent antibody fragment.

4. The recombinant proteinaceous binding molecule of item 3, wherein the divalent antibody fragment is an F(ab′)2-fragment, or a divalent single-chain Fv fragment.

5. The recombinant proteinaceous binding molecule of item 4, wherein the monovalent antibody fragment is selected from the group consisting of a Fv fragment, a single-chain Fv fragment (scFv) and a camelid single domain antibody.

6. The recombinant proteinaceous binding molecule of item 1, wherein the binding moiety having a first binding site for a first antigen is a binding molecule with antibody-like binding properties.

7. The recombinant proteinaceous binding molecule of item 6 wherein the binding molecules with antibody-like binding properties is selected from the group consisting of: an aptamer, an affilin, an affibody, an affimer, an atrimer, a polypeptide of the lipocalin family (anticalin), an adnectin, an avimer, an alphabody, an autofluorescent protein, a centyrin, a DARPin, a fynomer, a glubody, a kappabody, a Kringle domain, a Kunitz domain, a knottin, a nanofitin, a repebody, an antigen specific t-cell receptor, any receptor-protein, and any receptor-protein ligand.

8. The recombinant proteinaceous binding molecule of any one of the preceeding items, wherein the immunoglobulin CH3 domain of the first or the second heavy chain comprises the amino acid substitution T366W (CH3 knob-chain) and the immunoglobulin CH3 domain of the other heavy chain comprises at least one of the amino acid substitutions T366S, L368A and Y407V (CH3 hole-chain).

9. The recombinant proteinaceous binding molecule of item 8, wherein the CH3 hole-chain further comprises the amino acid substitution Y349C and the CH3-knob chain further comprises the amino acid substitution S354C.

10. The recombinant proteinaceous binding molecule of any one of the preceding items wherein at least one amino acid residue of the CH2 domain that is able to mediate binding to Fc receptors is lacking or mutated.

11. The recombinant proteinaceous binding molecule of item 10, wherein the amino acid residues are selected from the group consisting of sequence position 230, 231, 232, 233, 234, 235, 236, 237, 238, 265, 297, 327, and 330 (numbering of sequence positions according to the EU-index), wherein the least one mutation is preferably selected from the group consisting of a substitution Leu234->Ala, a substitution Leu235->Ala, a substitution Asn297->Ala, and a substitution Pro329->Ala.

12. The recombinant proteinaceous binding molecule of any one of the preceding items 1-11 wherein the first or the second heavy chain of the Fc fragment have a sequence identity of at least 80% to the sequence shown in SEQ ID NO: 7 (Fc-hole chain).

13. The recombinant proteinaceous binding molecule of item 12 wherein the CH-3 domain has a sequence identity of at least 80% to the sequence shown in SEQ ID NO: 29

14. The recombinant proteinaceous binding molecule of any one of the preceding items 1-13 wherein the first or the second heavy chain of the Fc fragment has a sequence identity of at least 80% to the sequence shown in SEQ ID NO: 8 (Fc-knob chain).

15. The recombinant proteinaceous binding molecule of item 15 wherein the CH-3 domain has a sequence identity of at least 80% to the sequence shown in SEQ ID NO: 30.

16. The recombinant proteinaceous binding molecule of any one of items 1-14, wherein the variable domain of and antibody light chain of the antibody variable domain of the second binding site for a second antigen is fused to the CH3 hole-chain

17. The recombinant proteinaceous binding molecule of item 17, wherein the variable domain of an antibody light chain of the second binding site for a second antigen fused to the CH3-hole chain has a sequence identity of at least 80% to the sequence shown in SEQ ID NO.:2.

18. The recombinant proteinaceous binding molecule of any one of items 1-14 wherein the variable domain of the antibody light chain of the second binding site for a second antigen is fused to the CH3 knob-chain.

19. The recombinant proteinaceous binding molecule of item 18, wherein the variable domain of an antibody light chain of the second binding site for a second antigen fused to the CH3 knob-chain has a sequence identity of at least 80% to the sequence shown in SEQ ID NO.:3.

20. The recombinant proteinaceous binding molecule of items 1-14, wherein the variable domain of an antibody heavy chain of the second binding site for a second antigen is fused to the CH3-hole chain.

21. The recombinant proteinaceous binding molecule of item 20 wherein the CH3-hole chain has a sequence identity of at least 80% to a sequence selected from the sequences shown in SEQ ID NO.:1 or SEQ ID NO.:10.

22. The recombinant proteinaceous binding molecule of any one of the preceding items, wherein the binding moiety having a first binding site for a first antigen fused to the CH3 knob-chain has a sequence identity of at least 80% to the sequence shown in SEQ ID NO.:4.

23. The recombinant proteinaceous binding molecule of any one of the preceding items, wherein the binding moiety having a first binding site for a first antigen fused to the CH3 knob-chain has a sequence identity of at least 80% to the sequence shown in SEQ ID NO.:5.

24. The recombinant proteinaceous binding molecule of any one of the preceding items, wherein the binding moiety having a first binding site for a first antigen fused to the CH3 knob-chain has a sequence identity of at least 80% to the sequence shown in SEQ ID NO.:6.

25. The recombinant proteinaceous binding molecule of any one of the preceding items, wherein the binding moiety having a first binding site for a first antigen fused to the CH3 hole-chain has a sequence identity of at least 80% to the sequence shown in SEQ ID NO.:9.

26. The recombinant proteinaceous binding molecule of any one of the preceding items, wherein the binding moiety having a first binding site for a first antigen fused to the CH3 knob-chain has a sequence identity of at least 80% to the sequence shown in SEQ ID NO.: 11.

27. The recombinant proteinaceous binding molecule of any one of the preceding items, wherein the first binding site binds a tumor-associated antigen.

28. The recombinant proteinaceous binding molecule of item 27, wherein the tumor associated antigen is located on the vasculature of a tumor.

29. The recombinant proteinaceous binding molecule of item 27 or 28, wherein the tumor associated antigen is a surface antigen or an antigen of the extracellular matrix.

30. The recombinant proteinaceous binding molecule of item 29, wherein the tumor associated antigen is selected from the group consisting of SlamF7, CD10, CD19, CD20, CD21, CD22, CD25, CD30, CD33, CD34, CD37, CD38, CD44v6, CD45, CDw52, CD70, CD117, CD123, CD133, CD135, CD138, CD140a, CD140b, CD171, CD309 CSPG4, Muc-1, Muc-16 Erb-B1, Erb-B2, Erb-B3, EGFRvIll, Folate receptor, PSMA, PSCA, PSA, VEGFR2, TAG-72, HLA-DR, IGFR, IL3R, fibroblast activating protein (FAP), CEA, EpCAM, CIaudin6, CLL-1, EphA10, G250, BB2, gp100, NY-ESO-1, LAGE-1, MAGE-A1, MAGE-A3, P-Cadherin, N-Cadherin, E-Cadherin, HLA-DP, HLA-A2, CCR4, CXCR3, FGFR1, GPC3, GPA33, GD2, BCMA, ROR1, TIM-3, Mesothelin (MSLN).

31. The recombinant proteinaceous binding molecule of any of items 1 to 30, wherein the second binding site of the variable domain of the antibody light chain or the variable domain of the antibody heavy chain binds a T-cell specific receptor molecule, a NK (natural killer) specific receptor molecule, a monocyte specific receptor molecule, a macrophage specific receptor molecule, a dendritic cell specific receptor molecule or a neutrophilic granulocyte cell specific receptor molecule.

32. The recombinant proteinaceous binding molecule of item 31, wherein the T-cell- or NK cell specific receptor molecule is one of CD3, the T cell receptor (TCR), CD28, CD16, NKG2D, 0x40, 4-1 BB, CD2, CD4, CD5, CD8, CD95., CD32a, CD64, CD89, NKp30, NKp40, PD1 CTLA4 or LFA1.

33. The recombinant proteinaceous binding molecule of item 32, wherein the TCR is TCR (alpha/beta), TCR (gamma/delta), CD3 gamma/epsilon or CD3 delta/epsilon.

34. The recombinant proteinaceous binding molecule of any of the preceding items, wherein the binding moiety is fused to the C- or N-terminus of the first or second heavy chain of the Fc fragment and the variable domain of either an antibody light chain or an antibody heavy chain is fused to the C- or N-terminus of the other heavy chain of the Fc fragment.

35. The recombinant proteinaceous binding molecule of item 34, further comprising a second binding moiety capable of binding an antigen, having a first binding site for a first antigen, wherein the second binding moiety is fused to the C- or N-terminus of the first or the second heavy chain of the Fc fragment.

36. The recombinant proteinaceous binding molecule of item 34 further comprising a second variable domain of either an antibody light chain or an antibody heavy chain of a second binding site for a second antigen, wherein the second variable domain of either an antibody light chain or an antibody heavy chain is fused to the C- or N-terminus of the first or the second heavy chain of the Fc fragment.

37. The recombinant proteinaceous binding molecule of item 36, further comprising a second binding moiety capable of binding an antigen, having a first binding site for a first antigen, wherein the second binding moiety is fused to the C- or N-terminus of the first or the second heavy chain of the Fc fragment.

38. A heterodimeric recombinant proteinaceous binding molecule comprising a heterodimer of recombinant proteinaceous binding molecules (monomers),

wherein the first monomer consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain meet each
other at an interface, which interface comprises an original interface between the CH3 domains,
wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and
wherein the variable domain of either the antibody light chain or the antibody heavy chain and the first binding moiety are linked via the Fc fragment,
wherein the second monomer consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface,
wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and
wherein the variable domain of either the antibody light chain or the antibody heavy chain and the first binding moiety are linked via the Fc fragment,
wherein the first antigen of the first monomer and the first antigen of the second monomer are two antigens of the same identity,
wherein the variable domain of an antibody light chain of the second binding site of the first monomer and the variable domain of an antibody heavy chain of the second binding site of the second monomer associate, thereby forming the second binding site and dimerizing the heterodimer.

39. The heterodimeric recombinant proteinaceous binding molecule of item 38, wherein the binding moiety which comprises a first binding site is an antibody fragment.

40. The heterodimeric recombinant proteinaceous binding molecule of item 39, wherein the antibody fragment is selected from the group consisting of a divalent antibody fragment or a monovalent antibody fragment.

41. The heterodimeric recombinant proteinaceous binding molecule of item 40, wherein the divalent antibody fragment is an (Fab)2′-fragment, or a divalent single-chain Fv fragment.

42. The heterodimeric recombinant proteinaceous binding molecule of item 40, wherein the monovalent antibody fragment is selected from the group consisting of a binding moiety, a Fv fragment, a single-chain Fv fragment (scFv) and a camelid single domain antibody.

43. The heterodimeric recombinant proteinaceous binding molecule of item 38, wherein the binding moiety having a first binding site for a first antigen is a binding molecule with antibody-like binding properties.

44. The heterodimeric recombinant proteinaceous binding molecule of item 43 wherein the binding molecule with antibody-like binding properties is selected from the group consisting of an aptamer, an affilin, an affibody, an affimer, an atrimer, a polypeptide of the lipocalin family (anticalin), an adnectin, an avimer, an alphabody, an autofluorescent protein, a centyrin, a DARPin, a fynomer, a glubody, a kappabody, a Kringle domain, a Kunitz domain, a knottin, a nanofitin, a repebody, an antigen specific t-cell receptor, any receptor-protein, and any receptor-protein ligand.

45. The heterodimeric recombinant proteinaceous binding molecule of item 38, wherein the immunoglobulin CH3 domain of the first or the second heavy chain comprises the amino acid substitution T366W (CH3 knob-chain) and the immunoglobulin CH3 domain of the other heavy chain comprises at least one of the amino acid substitutions T366S, L368A and Y407V (CH3 hole-chain).

46. The heterodimeric recombinant proteinaceous binding molecule of item 45, wherein the CH3 hole-chain further comprises the amino acid substitution Y349C and the CH3-knob chain further comprises the amino acid substitution S354C.

47. The heterodimeric recombinant proteinaceous binding molecule of any one of items 38 to 46, wherein at least one amino acid residue of the CH2 domains of each monomer that is able to mediate binding to Fc receptors is lacking or mutated.

48. The heterodimeric recombinant proteinaceous binding molecule of item 47, wherein the amino acid residues are selected from the group consisting of sequence 230, 231, 232, 233, 234, 235, 236, 237, 238, 265, 297, 327, and 330 (numbering of sequence positions according to the EU-index), wherein the least one mutation is preferably selected from the group consisting of a substitution Leu234->Ala, a substitution Leu235->Ala, a substitution Asn297->Ala, and a substitution Pro329->Ala.

49. The heterodimeric recombinant proteinaceous binding molecule of item 48, wherein the first binding site of each monomer binds a tumor associated antigen.

50. The heterodimeric recombinant proteinaceous binding molecule of item 49, wherein the tumor associated antigen is located on the vasculature of a tumor.

51. The heterodimeric recombinant proteinaceous binding molecule of item 49 or 50, wherein the tumor associated antigen is a surface antigen or an antigen of the extracellular matrix.

52. The heterodimeric recombinant proteinaceous binding molecule of item 51, wherein the tumor associated antigen is selected from the group consisting of SlamF7, CD10, CD19, CD20, CD21, CD22, CD25, CD30, CD33, CD34, CD37, CD38, CD44v6, CD45, CDw52, CD70, CD117, CD123, CD133, CD135, CD138, CD140a, CD140b, CD171, CD309 CSPG4, Muc-1, Muc-16 Erb-B1, Erb-B2, Erb-B3, EGFRvlll, Folate receptor, PSMA, PSCA, PSA, VEGFR2, TAG-72, HLA-DR, IGFR, IL3R, fibroblast activating protein (FAP), CEA, EpCAM, Claudin6, CLL-1, EphA10, G250, BB2, gp100, NY-ESO-1, LAGE-1, MAGE-A1, MAGE-A3, P-Cadherin, N-Cadherin, E-Cadherin, HLA-DP, HLA-A2, CCR4, CXCR3, FGFR1, GPC3, GPA33, GD2, BCMA, ROR1, TIM-3, Mesothelin (MSLN).

53. The heterodimeric recombinant proteinaceous binding molecule of any of items 38 to 52, wherein the second binding site of the variable domain of the antibody light chain or the variable domain of the antibody heavy chain binds a T-cell specific receptor molecule, a NK (natural killer) specific receptor molecule, a monocyte specific receptor molecule, a macrophage specific receptor molecule, a dendritic cell specific receptor molecule or a neutrophilic granulocyte cell specific receptor molecule.

54. The heterodimeric recombinant proteinaceous binding molecule of item 53, wherein the T-cell- or NK cell specific receptor molecule is one of CD3, the T cell receptor (TCR), CD28, CD16, NKG2D, 0x40, 4-1 BB, CD2, CD4, CD5, CD8 CD95, CD32a, CD64, CD89, NKp30, NKp40, PD1 CTLA4 or LFA1, or wherein the dendritic cell specific receptor molecule is CD40.

55. The heterodimeric recombinant proteinaceous binding molecule of item 54 wherein the TCR is TCR (alpha/beta) or TCR (gamma/delta) CD3 gamma/epsilon or CD3 delta/epsilon.

56. The heterodimeric recombinant proteinaceous binding molecule of any of items 38 to 55 wherein the Binding moiety of each monomer is fused to the C- or N-terminus of the first or the second heavy chain of the Fc fragment and the variable domain of either an antibody light chain or an antibody heavy chain is fused to the C- or N-terminus of the other heavy chain of the Fc fragment.

57. The heterodimeric recombinant proteinaceous binding molecule of item 43, wherein each monomer further comprises a second binding moiety capable of binding an antigen, having a first binding site for a first antigen, wherein the second binding moiety is fused to the C- or N-terminus of the first or the second heavy chain of the Fc fragment.

58. The heterodimeric recombinant proteinaceous binding molecule of item 56 wherein each monomer further comprises a second variable domain of either an antibody light chain or an antibody heavy chain of a second binding site for a second antigen, wherein the second variable domain of either the light chain or the heavy chain is fused to the C- or N-terminus of the first or the second heavy chain of the Fc fragment.

59. The recombinant proteinaceous binding molecule of item 58, wherein each monomer further comprises a second binding moiety capable of binding an antigen, having a first binding site for a first antigen, wherein the second binding moiety is fused to the C- or N-terminus of the first or the second heavy chain of the Fc fragment.

60. A heterodimeric recombinant proteinaceous binding molecule comprising a heterodimer of recombinant proteinaceous molecules (monomers),

wherein the first monomer consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, which interface comprises an original interface between the CH3 domains,
wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and
wherein the variable domain of either the antibody light chain or the antibody heavy chain and the first binding moiety are linked via the Fc fragment,
wherein the second monomer consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface,
wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and
wherein the variable domain of either the antibody light chain or the antibody heavy chain and the first binding moiety are linked via the Fc fragment,
wherein the first antigen of the first monomer and the first antigen of the second monomer are two antigens of different identity,
wherein the variable domain of an antibody light chain of the second binding site of the first monomer and the variable domain of an antibody heavy chain of the second binding site of the second monomer associate thereby forming the second binding site and dimerizing the heterodimer.

61. The heterodimeric recombinant proteinaceous binding molecule of item 60, wherein the binding moiety having a first binding site for a first antigen is an antibody fragment.

62. The heterodimeric recombinant proteinaceous binding molecule of item 61, wherein the antibody fragment is selected from the group consisting of a divalent antibody fragment, and a a monovalent antibody fragment.

63. The heterodimeric recombinant proteinaceous binding molecule of item 62, wherein the divalent antibody fragment is an (Fab)2′-fragment, or a divalent single-chain Fv fragment.

64. The heterodimeric recombinant proteinaceous binding molecule of item 62 wherein the monovalent antibody fragment is selected from the group consisting of a Binding moiety, a Fv fragment, a single-chain Fv fragment (scFv) and a camelid single domain antibody.

65. The heterodimeric recombinant proteinaceous binding molecule of item 60 wherein the binding moiety comprising a first binding site for a first antigen is a binding molecule with antibody-like binding properties.

66. The heterodimeric recombinant proteinaceous binding molecule of item 65 wherein the binding molecule with antibody-like binding properties is selected from the group consisting of: an aptamer, an affilin, an affibody, an affimer, an atrimer, an anticalin, an adnectin, an avimer, an alphabody, an autofluorescent protein, a centyrin, a DARPin, a fynomer, a glubody, a kappabody, a Kringle domain, a Kunitz domain, a knottin, a nanofitin, a repebody, an antigen specific t-cell receptor, any receptor-protein, any receptor-protein ligand.

67. The heterodimeric recombinant proteinaceous binding molecule of item 60, wherein the immunoglobulin CH3 domain of the first or the second heavy chain comprises the amino acid substitution (CH3 knob-chain) and the immunoglobulin CH3 domain of the other heavy chain comprises at least one of the amino acid substitutions T366S, L368A and Y407V (CH3 hole-chain).

68. The heterodimeric recombinant proteinaceous binding molecule of item 67, wherein the CH3 hole-chain further comprises the amino acid substitution Y349C and the CH3-knob chain further comprises the amino acid substitution S354C.

69. The heterodimeric recombinant proteinaceous binding molecule of item 60 to 68 wherein at least one amino acid residue of the CH2 domains of each monomer that is able to mediate binding to Fc receptors is lacking or mutated.

70. The heterodimeric recombinant proteinaceous binding molecule of item 69 wherein the amino acid residues are selected from the group consisting of sequence position 230, 231, 232, 233, 234, 235, 236, 237, 238, 265, 297, 327, and 330 (numbering of

sequence positions according to the EU-index), wherein the least one mutation is preferably selected from the group consisting of a substitution Leu234->Ala, a substitution Leu235->Ala, and a substitution Asn297->Ala.

71. The heterodimeric recombinant proteinaceous binding molecule of item 60, wherein the first binding site of the first monomer binds a tumor associated antigen and wherein the third binding site of the second monomer binds a tumor associated antigen.

72. The heterodimeric recombinant proteinaceous binding molecule of item 71, wherein the tumor associated antigen is located on the vasculature of a tumor.

73. The heterodimeric recombinant proteinaceous binding molecule of item 71 or 72, wherein the tumor associated antigen is a surface antigen or an antigen of the extracellular matrix.

74. The heterodimeric recombinant proteinaceous binding molecule of item 73, wherein the tumor associated antigen is selected from the group consisting of SlamF7, CD10, CD19, CD20, CD21, CD22, CD25, CD30, CD33, CD34, CD37, CD38, CD44v6, CD45, CDw52, CD70, CD 117, CD123, CD133, CD135, CD138, CD140a, CD140b, CD171, CD309 CSPG4, Muc-1, Muc-16 Erb-B1, Erb-B2, Erb-B3, EGFRvIll, Folate receptor, PSMA, PSCA, PSA, VEGFR2, TAG-72, HLA-DR, IGFR, IL3R, fibroblast activating protein (FAP), CEA, EpCAM, CIaudin6, CLL-1, EphA10, G250, BB2, gp100, NY-ESO-1, LAGE-1, MAGE-A1, MAGE-A3, P-Cadherin, N-Cadherin, E-Cadherin, HLA-DP, HLA-A2, CCR4, CXCR3, FGFR1, GPC3, GPA33, GD2, BCMA, ROR1, TIM-3, Mesothelin (MSLN).

75. The heterodimeric recombinant proteinaceous binding molecule of any of items 60 to 73 wherein the second binding site of the variable domain of the antibody light chain or the variable domain of the antibody heavy chain binds a T-cell specific receptor molecule, a NK (natural killer) specific receptor molecule, a monocyte specific receptor molecule, a macrophage specific receptor molecule, a dendritic cell specific receptor molecule or a neutrophilic granulocyte cell specific receptor molecule.

76. The heterodimeric recombinant proteinaceous binding molecule of item 75, wherein the T-cell- or NK cell specific receptor molecule is one of CD3, the T cell receptor (TCR), CD28, CD16, NKG2D, 0x40, 4-1 BB, CD2, CD 4, CD5, CD8 and CD95, or wherein the dendritic cell specific receptor molecule is CD40.

77. The heterodimeric recombinant proteinaceous binding molecule of item 76, wherein the TCR is TCR (alpha/beta) or TCR (gamma/delta) CD3 gamma/epsilon or CD3 delta/epsilon.

78. The heterodimeric recombinant proteinaceous binding molecule of any of items 60 to 77, wherein the binding moiety of each monomer is fused to the C- or N-terminus of the first or the second heavy chain of the Fc fragment and the variable domain of either the light chain or the heavy chain is fused to the C- or N-terminus of the other heavy chain of the Fc fragment.

79. The heterodimeric recombinant proteinaceous binding molecule of item 78, wherein each monomer further comprises a second binding moiety capable of binding an antigen, having a first binding site for a first antigen, wherein the second binding moiety is fused to the C- or N-terminus of the first or the second heavy chain of the Fc fragment.

80. The heterodimeric recombinant proteinaceous binding molecule of items 60 to 77 wherein each monomer further comprises a second variable domain of either an antibody light chain or an antibody heavy chain of a second binding site for a second antigen, wherein the second variable domain of either an antibody light chain or an antibody heavy chain is fused to the C- or N-terminus of the first or the second heavy chain of the Fc fragment.

81. The recombinant proteinaceous binding molecule of item 80, wherein each monomer further comprises a second binding moiety capable of binding an antigen, having a first binding site for a first antigen, wherein the second binding moiety is fused to the C- or N-terminus of the first or the second heavy chain of the Fc fragment.

82. A pharmaceutical composition comprising a recombinant proteinaceous binding molecule as defined in any one of the preceding items.

83. A recombinant proteinaceous binding molecule or a heterodimeric recombinant proteinaceous binding molecule as defined in any of items 1 to 81 for use in the treatment or diagnosis of a disease.

84. The recombinant proteinaceous binding molecule or a heterodimeric recombinant proteinaceous binding molecule of item 83 wherein the disease is a proliferatory disease.

85. The recombinant proteinaceous binding molecule or a heterodimeric recombinant proteinaceous binding molecule of item 84, wherein the proliferatory disease is selected from the group consisting of hematopoietic malignancies, such as acute and chronic myeloic and lymphatic leukemias, as well as lymphomas, solid tumors such as tumors of the gastrointestinal tract, lung, kidney, prostate, breast, brain, ovary, uterus, mesenchymal tumors and melanoma.

86. A nucleic acid molecule encoding a recombinant proteinaceous binding molecule or a heterodimeric recombinant proteinaceous binding molecule as defined in any of items 1 to 81.

87. A nucleic acid molecule of item 86 comprised in a vector.

88. A host cell comprising a nucleic acid molecule of item 86 or a vector of item 87.

89. A method of producing recombinant proteinaceous binding molecule of any one of items 1 to 85, comprising expressing a nucleic acid encoding the recombinant proteinaceous binding molecule under conditions allowing expression of the nucleic acid.

90. The method of item 89 wherein the recombinant proteinaceous binding molecule is expressed in a host cell or a cell-free system.

91. The use of a recombinant proteinaceous binding molecule of any one of items 1 to 81 for the treatment of a disease, wherein the recombinant proteinaceous binding molecule forms a heterodimer only in vivo on a target cell, thereby reducing “off target activation”

Furthermore, the present invention is further characterized by the following items:

1. A recombinant proteinaceous binding molecule comprising.
a) a first binding moiety, capable of binding an antigen, having a first binding site for a first antigen,
b) a variable domain of either an antibody light chain or an antibody heavy chain of a second binding site for a second antigen, and
c) an Fc fragment comprising a first and a second heavy chain,
wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, which interface comprises an original interface between the CH3 domains,
wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and
wherein the variable domain of either the antibody light chain or the antibody heavy chain and the first binding moiety are linked via the Fc fragment.

2. The recombinant proteinaceous binding molecule of item 1, wherein the binding moiety having a first binding site is an antibody fragment.

3. The recombinant proteinaceous binding molecule of item 2, wherein the antibody fragment is selected from the group consisting of a divalent antibody fragment, and a monovalent antibody fragment, wherein the divalent antibody fragment is preferably an F(ab′)2-fragment, or a divalent single-chain Fv fragment, wherein the monovalent antibody

fragment is selected from the group consisting of a Binding moiety, a Fv fragment, a single chain Fv fragment (scFv) and a camelid single domain antibody.

4. The recombinant proteinaceous binding molecule of item 1, wherein the binding moiety having a first binding site is a binding molecule with antibody-like binding properties.

5. The recombinant proteinaceous binding molecule of item 3 wherein the binding molecules with antibody-like binding properties is selected from the group consisting of: an aptamer, an affilin, an affibody, an affimer, an atrimer, an anticalin, an adnectin, an avimer, an alphabody, an autofluorescent protein, a centyrin, a DARPin, a fynomer, a glubody, a kappabody, a Kringle domain, a Kunitz domain, a knottin, a nanofitin, a repebody, an antigen specific t-cell receptor, any receptor-protein, any receptor-protein ligand.

6. The recombinant proteinaceous binding molecule of item 1, wherein the immunoglobulin CH3 domain of the first heavy chain comprises at least one of the amino acid substitutions T366S, L368A and Y407V (CH3 hole-chain) and the immunoglobulin CH3 domain of the second heavy chain comprises the amino acid substitution T366W (CH3 knob-chain), wherein the CH3 hole-chain further comprises the amino acid substitution Y349C and the CH3-knob chain further comprises the amino acid substitution S354C.

7. The recombinant proteinaceous binding molecule of any one of the preceding items wherein at least one amino acid residue of the CH2 domain that is able to mediate binding to Fc receptors is lacking or mutated, wherein the amino acid residues are selected from the group consisting of sequence position 230, 231, 232, 233, 234, 235, 236, 237, 238, 265, 297, 327, and 330 (numbering of sequence positions according to the EU-index), wherein the least one mutation is preferably selected from the group consisting of a substitution Leu234->Ala, a substitution Leu235->Ala, a substitution Asn297->Ala, and a substitution Pro329->Ala.

8. The recombinant proteinaceous binding molecule of any one of the preceding items, wherein the first binding site binds a tumor-associated antigen.

9. The recombinant proteinaceous binding molecule of any one of the preceding items, wherein the light chain of the variable fragment of the second binding site fused to the CH3-hole chain may have sequence identity of at least 80% to the sequence shown in SEQ ID NO.:2, or wherein the light chain of the variable fragment of the second binding site fused to the CH3-knob chain may have sequence identity of at least 80% to the sequence shown in SEQ ID NO.:3, or wherein the heavy chain of the variable fragment of the second binding site fused to the CH3-hole chain may have sequence identity of at least 80% to the sequence selected from the sequences shown in SEQ ID NO.:1 or SEQ ID NO.: 10.

10. The recombinant proteinaceous binding molecule of any one of the preceding items, wherein the binding moiety having a first binding site fused to the CH3-knob chain may have sequence identity of at least 80% to the sequence shown in SEQ ID NO.:4, or wherein the binding moiety having a first binding site fused to the CH3-knob chain may have sequence identity of at least 80% to the sequence shown in SEQ ID NO.:5, or wherein the binding moiety having a first binding site fused to the CH3-knob chain may have sequence identity of at least 80% to the sequence shown in SEQ ID NO.:6, or wherein the binding moiety having a first binding site fused to the CH3-hole chain may have sequence identity of at least 80% to the sequence shown in SEQ ID NO.:9, or wherein the binding moiety having a first binding site fused to the CH3-knob chain may have sequence identity of at least 80% to the sequence shown in SEQ ID NO.: 11

11. The recombinant proteinaceous binding molecule of any of items 1 to 10, wherein the light chain or the heavy chain of a second binding site for a second antigen are a light chain or a heavy chain of a second binding site for a T-cell, NK (natural killer), Monocyte, Macrophage, Dendritic Cell or Neutrophilic Granulocyte cell specific receptor molecule (CD3, the T cell receptor (TCR), CD28, CD16, NKG2D, 0x40, 4-1 BB, CD2, CD4, CD5, CD8, CD95, CD32a, CD40, CD89, CD64, NKp30, NKp40, PD1, CTLA4, LFA1).

12. A heterodimeric recombinant proteinaceous binding molecule comprising a heterodimer of recombinant proteinaceous molecules (monomers),
wherein the first monomer consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, which interface comprises an original interface between the CH3 domains
   wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
   wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and
   wherein the variable domain of either the antibody light chain or the antibody heavy chain and the first binding moiety are linked via the Fc fragment,
   wherein the second monomer consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain each meet each other at an interface,
   wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
   wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and
   wherein the variable domain of either the antibody light chain or the antibody heavy chain and the first binding moiety are linked via the Fc fragment,
   wherein the first antigen of the first monomer and the first antigen of the second monomer are two antigens of different identity,
wherein the variable domain of an antibody light chain of the second binding site of the first monomer and the variable domain of an antibody heavy chain of the second binding site of the second monomer associate thereby forming the second binding site and dimerizing the heterodimer.

13. A pharmaceutical composition comprising a recombinant proteinaceous binding molecule as defined in any of the preceding items.

14. A recombinant proteinaceous binding molecule as defined in any of items 1 to 11 for use in the treatment or diagnosis of a disease, wherein the disease is preferably a proliferative disease.

15. The use of a recombinant proteinaceous binding molecule of any one of items 1 to 11 for the treatment of a disease, wherein the recombinant proteinaceous

binding molecule forms a heterodimer only in vivo on a target cell, thereby reducing “off target activation”.

### EXAMPLES

### Example 1—Design, expression and purification of KiHss hemibody constructs

All DNA sequences that were used for cloning have been generated by de novo DNA synthesis (GeneArt Gene Synthesis, Thermo Fisher Scientific, USA). Complete hemibody coding sequences were cloned into the piggybac transposon expression vector system PB514B-1 (System Biosciences, USA) via the Nhel and Notl restriction sites. Alternatively, coding sequences of the Knob into to hole constructs were cloned into pCET1019AS-puro and pCET1019AS-hygro vectors (Merck-Millipore, USA) via the restriction sites NgoMIV and Bmtl. Sequence identity of the final expression vectors was confirmed by Sanger sequence analysis. The cloned plasmid vectors were used to generate stable expression cell lines. In case of the transposon-based expression vectors Expi293F cells (Thermo Fisher Scientific, USA) were co-transfected with the vectors encoding the Knob and the Hole subunit as well as the piggybac transposase vector PB210PA-1 (System Biosciences, USA) using the ExpiFectamine™ 293 Transfection Kit (Thermo Fisher Scientific, USA) whereas the pCET vectors were 1-Scel linerarized and transferred into Freestyle CHO-S cells (Thermo Fisher Scientific, USA) using Lipofectamine 2000 (Thermo Fisher Scientific, USA). Handling and transfection of the Expi293F and CHO-S cells was done according to the instructions provided by the manufacturer. To obtain stable expression cell lines the transfected cells were selected using puromycin (Invivogen, USA) at a final concentration of 5 pg/mL (Expi293F), 1.5-3 pg/mL (CHO-S) or 250-500 pg/mL hygromycin B (Thermo Fisher Scientific, USA) over a period of at least two weeks in shaking flasks. For sub-cultivation and for production Expi293F cells were maintained in Freestyle™ 293 Expression Medium supplemented with 1:500 diluted Gibco anti-clumping agent (Thermo Fisher Scientific, USA) and 5 pg/mL puromycin. CHO-S cells were cultured in HyClone ActiSM or ActiPro (Cytiva, USA) medium supplemented with 4 mM L-glutamine (Thermo Fisher Scientific, USA). For production stable expression cell lines were seeded at a concentration of 0.3-0.5×10*6 cells/mL. The culture was harvested when cell viability was between 70-60%. Subsequently the cells were separated from culture supernatant by a repeated centrifugation step at 2000×g for 15 min. The clarified supernatant was sterilized through filtration at a filter pore size of 1.2 pm. To isolate the hemibody constructs the culture supernatant was first dialyzed (MWCO 14 kDa) twice at a sample to buffer ratio of 1:20 to 1:25 using a 25 mM Na-phosphate pH7.5, 150 mM NaCl solution as dialysis buffer and then subjected to immobilized metal affinity chromatography (IMAC) using the AKTApure system (Cytiva, USA) and a

HiTrap TALON column (Cytiva, USA) for construct capture. For column washing a 50 mM Na-phosphate pH7.5, 300 mM NaCl solution and for elution a 50 mM Na-phosphate pH7.5, 300 mM NaCl, 200 mM Imidazole pH8.0 solution was used. To remove impurities and to obtain monomeric hemibody species the IMAC processed constructs were further purified on a Superdex 200 Increase 10/300 GL size exclusion chromatography (SEC) column (Cytiva, USA) with 50 mM Na-phosphate pH7.5, 300 mM NaCl solution as the size exclusion buffer at a volumetric flow rate of 0.4 mL/min. FIG. 2 shows the electrophoretic separation of the purified KiHss hemibody Constructs V L antiEpcam, VH antiEpcam, VL antiEGFR, VH antiEGFR and VL antiHer2. The sequence numbers and the size of said constructs are summarized in FIG. 24.

Yield and purity of the hemibody constructs obtained after the size exclusion purification step as depicted in FIG. 2 are shown in the Table below.

| | Yield (mg/L | |
|---|---|---|
| Construct | culture broth) | Purity [%] |
| VL antiEpcam | 1.29 | 95-100 |
| VH antiEpcam | 0.57 | 95-100 |
| VL antiEGFR | 0.32 | 95-100 |
| VH antiEGFR | 0.14 | 95-100 |
| VL antiHer2 | 0.16 | 95-100 |

The SEC elution-fractions containing the monomeric hemibody construct were pooled, 0.2 pm sterile-filtered, stored at 2-8° C. and used for all following experiments. Using this technique KiHss hemibodies constructs shown in FIG. 24 were produced successfully.

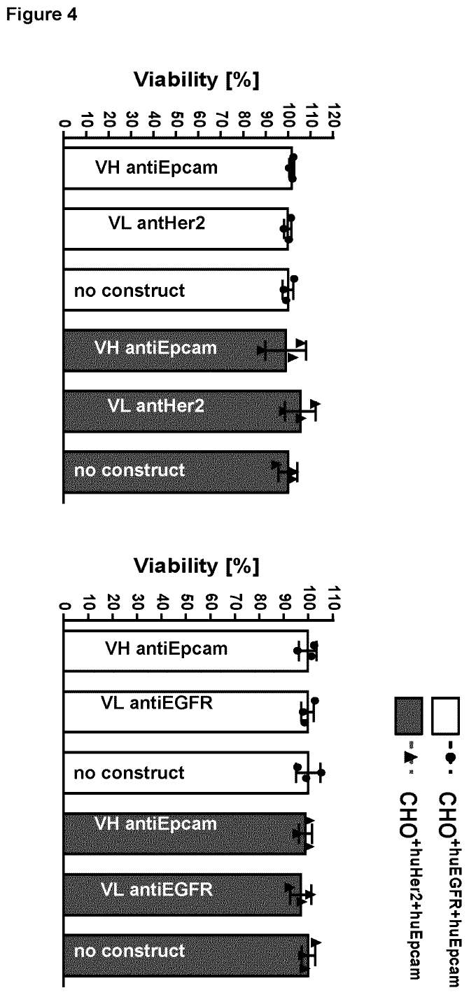

### Example 2—Target-Cell Lysis (Corresponds to FIGS. 3 and 4)

KiHss hemibodies trigger a dose dependent target-specific destruction of dual target-antigen positive cells by activation and retargeting of cytotoxic T-cell, whereas single target-antigen positive cells are excluded from lysis. Combinatorial antigen specific cell lysis was achieved by co-cultivation of CHO cells co-expressing the target-antigens and CD8 positive T-cells in the presence of increasing amounts of target specific hemibodies (FIG. 3). In the presence of the hemibody pair VH antiEpcam+VL antiHer2 (FIG. 3 A) only cells which are positive for Epcam and Her2 were precisely lysed at a concentration of up to 1 nM. In contrast the KiHss hemibody pair VH antiEpcam+VL antiEGFR (FIG. 3 B) triggered a specific lysis of cells positive for Epcam and EGFR in the range of 0.02 nM to 1 nM. To test if single hemibodies can elicit a T-cell dependent target cell lysis, antigen overexpressing CHO cells and cytotoxic T-cells were co-cultured in the presence of single hemibody constructs at a concentration of 5 nM (FIG. 4). At the given concentration single hemibody constructs neither triggered target cell lysis nor affect cell viability. For target-cell lysis experiments 10,000 target-antigen and firefly luciferase (Flue) co-expressing Chinese hamster ovary cells (CHO K1, DSMZ ACC-110) were seeded per well of a 96 well plate (flat white Costar, Corning, USA) in 0.05 ml culture medium. After seeding, the plates were incubated at room temperature for 1 h and then further incubated at standard cell culture conditions (37° C., 5% C02) for 20 h. The next day 35,000 CD8 positive T-cells isolated from human PBMCs were added per well in 0.05 ml culture medium to give a final volume of 0.1 ml. After adding diluted hemibody constructs cells were further incubated at standard cell culture conditions for 20 h. Intracellular luciferase activity was monitored to determine lysis of the firefly luciferase expressing CHO cells in the presence of CD8 positive T cells and hemibody constructs. To this end, D-Luciferin (Biosynth, USA) was added to a final concentration of 0.5 mM and incubated at 37° C. for 20-30 min. Subsequently, light emission was quantified with the infinite M200 pro ELISA reader (Tecan, Switzerland). Total cell killing corresponds to 100%. All assay values were statistically evaluated with the GraphPad Prism6 software (Graphpad Software, USA).

$$Calculation of cell viability \frac{{RLI}_{sample} \times 100 \%}{{RLI}_{control}} = Viability [ \% ]$$

$$Calculation of specific lysis 100 - Viability [ \% ] = \% specific lysis$$

$$Legend$$

$$RLI : relative luminescence intensity$$

$$Sample : Effector cells + target cells + hemibody$$

$$Control : Effector cells + target cells$$

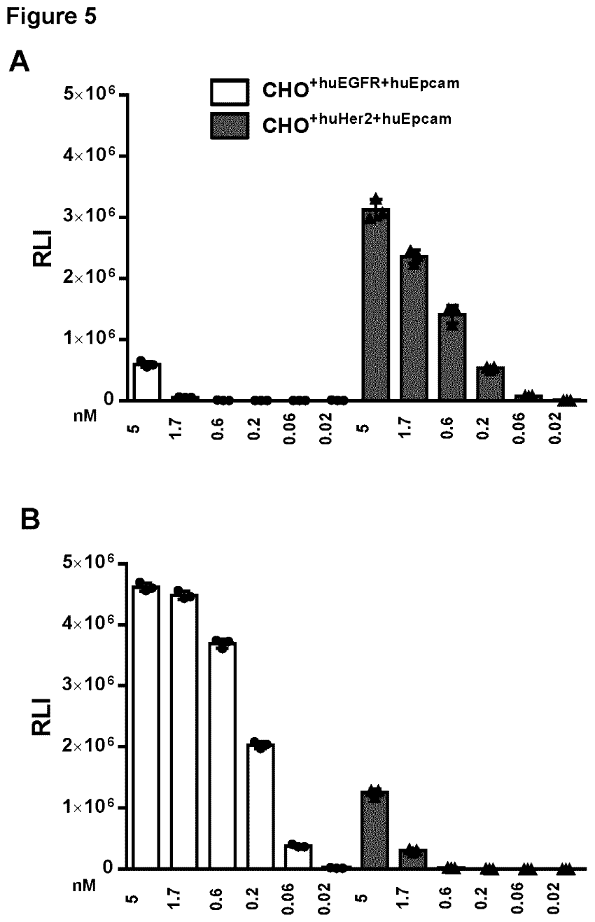

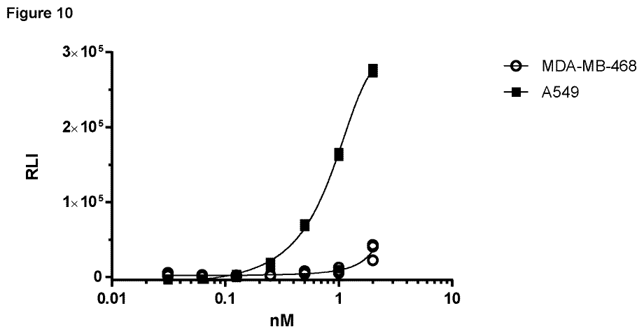

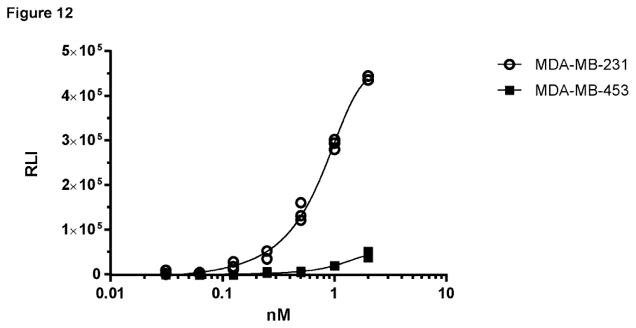

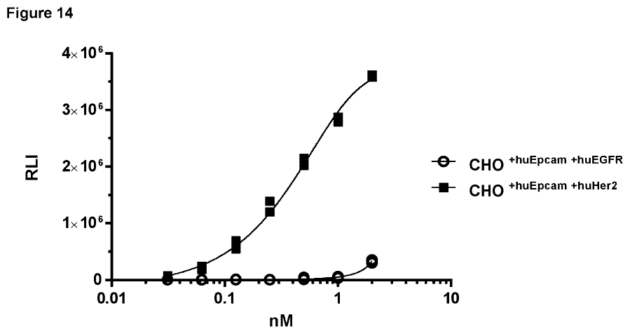

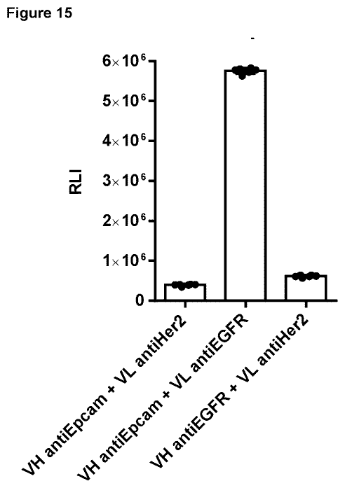

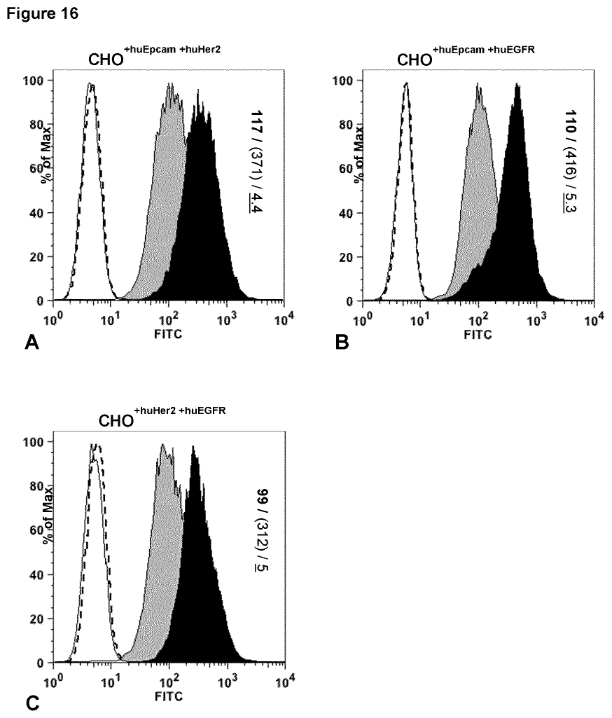

### Example 3—T-cell activation (corresponds to FIGS. 5, 6, 7, 8, 9, 10, 12, 14 and 15, 35 and 36)

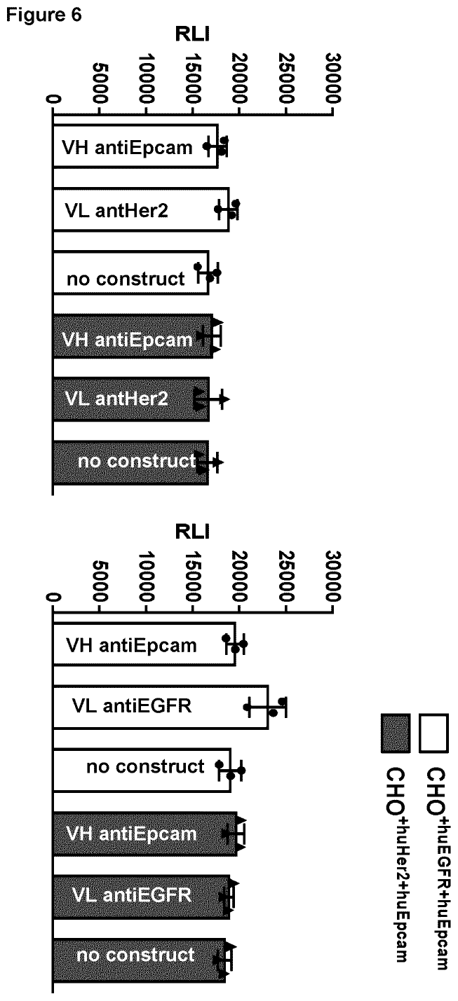

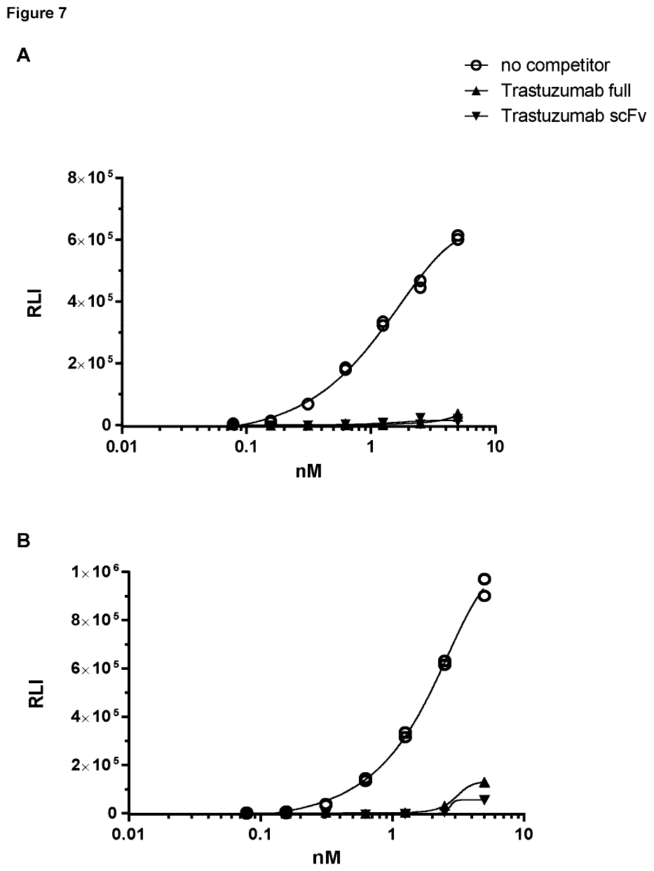

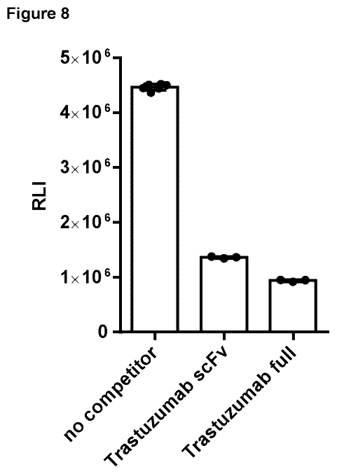

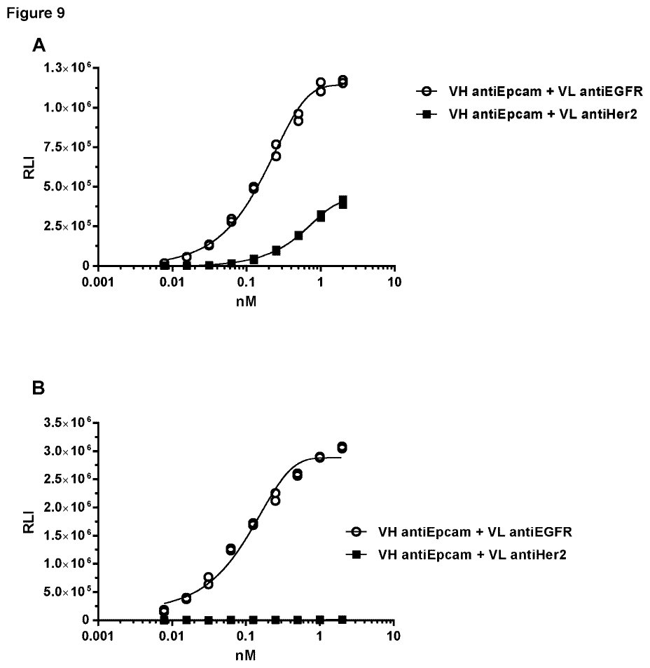

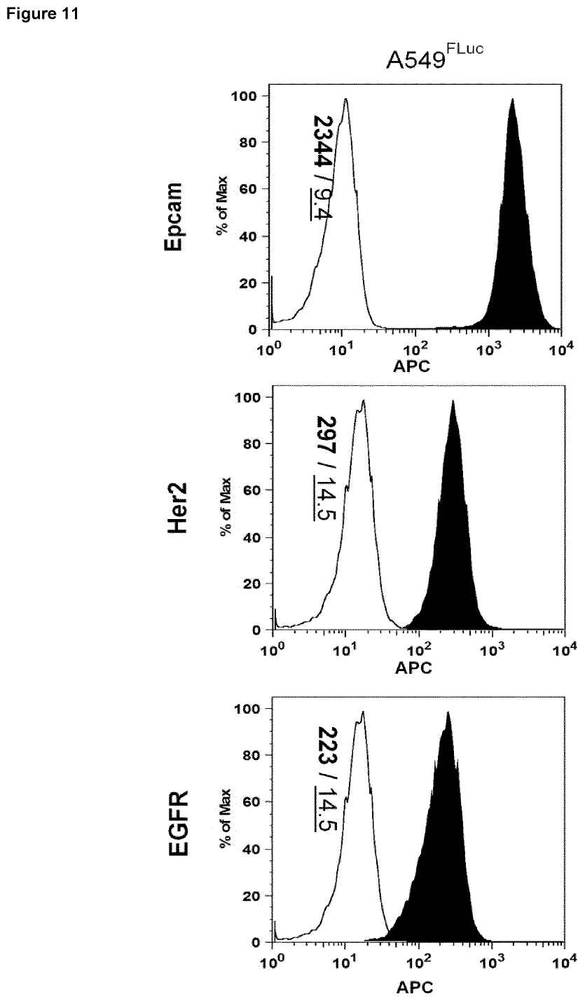

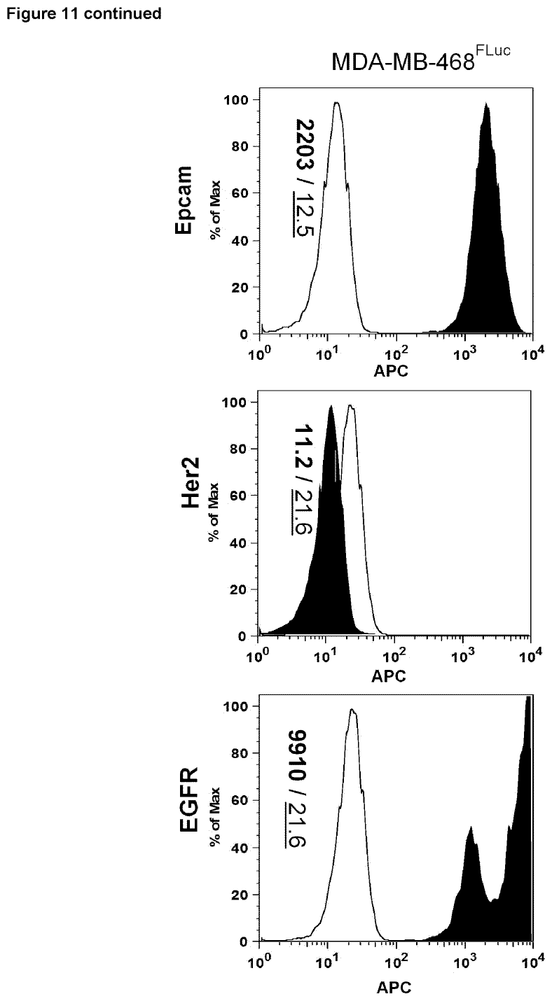

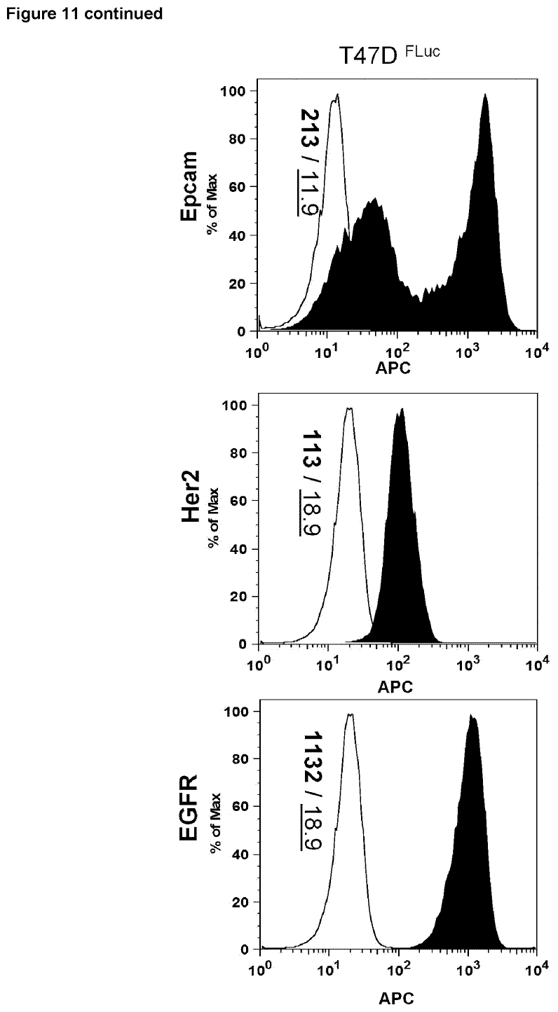

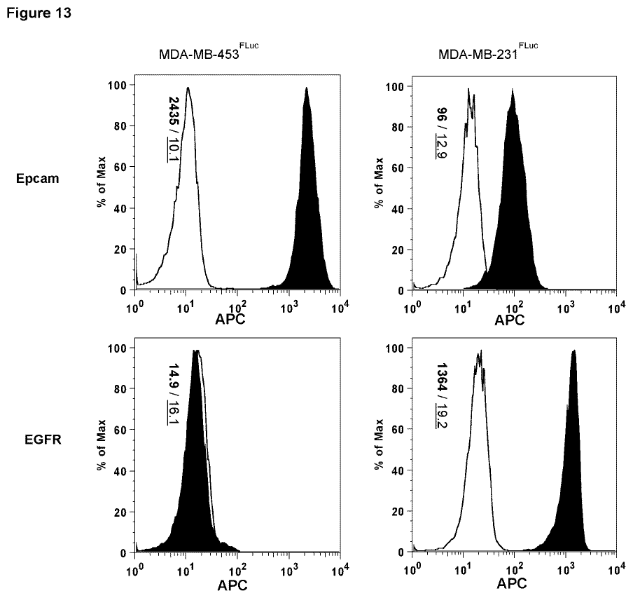

Hemibodies in the KiHss format induce a dose and target dependent activation of T-cells with picomolar potency. For assaying hemibody triggered T-cell activation a Jurkat based reporter cell line, which was engineered by stable integration of an NFAT-inducible reporter construct encoding a secreted coelenterazine-utilizing luciferase was used. Binding of CD3 specific TCR activators stimulate a dose dependent NFAT controlled luciferase expression. The level of secreted luciferase activity is a direct measure for activation of the T-cell. As it is shown in FIGS. 5, 14, 15, 35 and 36 by co-cultivation of Jurkat reporter cells and target-antigen expressing CHO cells in the presence of increasing amounts of hemibodies directed against the respective target-combination a dose dependent and combination specific T-cell activation was achieved. Moreover, as indicated by the absence of luciferase activity KiHss hemibodies did not raise a specific T-cell response when applied as single constructs (FIG. 6). Dose dependency of hemibody triggered T-cell activation was confirmed by using established human tumor cell lines characterized by a defined target-antigen expression profile as verified by flow cytometric (FIGS. 11 and 13). As shown in FIGS. 9, 10 and 12 availability of hemibodies led to a robust, target specific and concentration dependent activation of the recombinant Jurkat T cells when incubated together with target cells. To further assess the specificity of hemibody induced T-cell activation target-antigen blocking experiments were performed (FIGS. 7 and 8). For target-antigen blockage the antibody Trastzuzumab was used as Her2 specific competitor in the full antibody as well as in the scFv format. The tumor cell lines T47D, A549 and CHO cells co expressing Epcam and Her2 were employed as target cells. As seen in FIGS. 7 and 8, the hemibody mediated activation of Jurkat T-cells was inhibited by supplementation with a molar excess of the Her2 specific competitor, demonstrating the efficient and specific hemibody assisted targeting of tumor cells.

For T-cell activation experiments 10,000 target-antigen and firefly luciferase (Flue) co-expressing Chinese hamster ovary cells (CHO K1, DSMZ ACC-110), MDA-MB-231 (DSMZ ACC-732), MDA-MB-453 (DSMZ ACC-65), MDA-MB-468 (DSMZ ACC-738), A549 (DSMZ ACC-107), T47D (DSMZ ACC-739), HT29 (DSMZ ACC-299) or HCT1116 (DSMZ ACC-581) were seeded per well on a 96 well plate (flat transparent Costar, Corning, USA) in 0.05 ml cell culture medium. After seeding the plates were incubated at room temperature for 1 h and then further incubated at standard cell culture conditions (37° C., 5% C02) for 20 h. The next day 50,000 Jurkat Lucia NFAT cells (Cat #jktl-nfat, InvivoGen) resuspended in 0.05 ml cell culture medium were added per well to give a final volume of 0.1 ml. After adding the hemibody constructs cells were further incubated at standard cell culture conditions for 20 h. Secreted luciferase was monitored to determine activation of the Jurkat Lucia NFAT cells in the presence of target cells and hemibody constructs. To this end, 0.04 ml cell culture supernatant were mixed 0.04 ml PBS supplemented with 5 mM native Coelenterazine (Biosynth, USA). Subsequently, relative luminescence intensity (RLI) was quantified with the infinite M200 pro ELISA reader (Tecan, Switzerland). In case of target-antigen competition experiments (corresponds to FIGS. 7 and 8), before addition of hemibody constructs and after combining Jurkat Lucia NFAT cells and target cells the culture was pre-incubated with target-antigen competitor for 2 h at standard cell culture conditions. All assay values were statistically evaluated with the GraphPad Prism6 software (Graphpad Software, USA). The Jurkat Lucia NFAT cells and the tumor cell lines were maintained in Gibco Advanced RPMI1640 medium (Thermo Fisher Scientific, USA) supplemented with 10% Gibco H I FBS (Thermo Fisher Scientific, USA), 1:100 Gibco Glutamax (Thermo Fisher Scientific, USA) and 1:100 Gibco PSN (Thermo Fisher Scientific, USA) whereas the CHO cells were grown in Gibco Ham's F-12K (Kaighn's) Medium (Thermo Fisher Scientific, USA) supplemented with 10% Gibco HI FBS (Thermo Fisher Scientific, USA), 1:100 Gibco Glutamax (Thermo Fisher Scientific, USA) and 1:100 Gibco PSN (Thermo Fisher Scientific, USA).

### Example 4—Determination of target-antigen expression status of target cells, (corresponds to FIGS. 11, 13, 16, 20, 35 and 36)

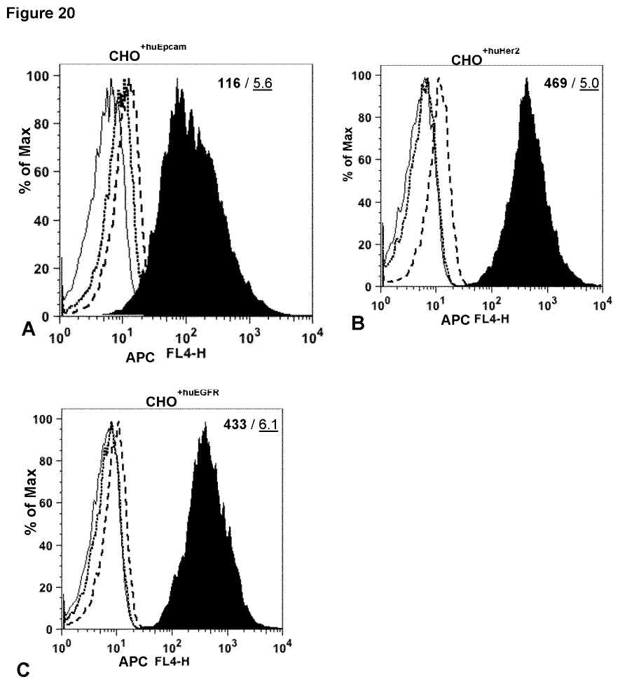

In order to validate the employed cell lines for use as target cells in tumor lysis and T cell activation experiments, the target-antigen expression profile was defined by flow cytometry. As shown in FIGS. 11 and 13 the human tumor cell lines A549 and T47D were positive for Epcam, EGFR and Her2, the MDA-MB-453 cell line was shown to be strictly negative for EGFR while displaying a strong Epcam expression. In contrast MDA-MB-468 cells revealed a high EGFR and Epcam surface density whereas Her2 expression was lacking. The triple negative breast cancer cell line MDA-MB-231 revealed a high EGFR and a median Epcam expression rate. As shown in FIG. 37, the human colon cancer cell lines HT29 and HCT116 were positive for EGFR, Her2 and Epcam. HT29 cell line was also shown to be positive for ROR1 whereas ROR1 expression was extremely low in HCT116 cells. Furthermore, the assigned antigen expression profile of the engineered CHO cell clones was confirmed by using target specific antibodies, as seen from FIGS. 16 and 20.

To quantify the antigen expression level the adherent target cells were detached from culture plate using Gibco 0.05% trypsin-EDTA solution (Thermo Fisher Scientific, USA). Cells were then resuspended in FACS-buffer (PBS supplemented with 1% FBS) to a concentration of 1×10*6/ml_and incubated with fluorophore conjugated target specific antibodies (20 pl_/1*×10*6 cells) for 30 min. Before cells were subjected to flow cytometry excessive antibody was removed by washing. For fluorophore signal quantification we used the FACS Calibur cytometer (BD Biosiences, USA). For staining we used the following antibodies: APC antihuHer2, Cat. No. 324407 (Biolegend, USA); APC antihuEGFR, Cat. No.

352905 (Biolegend, USA); APC antihuEpcam, Cat. No. 324207 (Biolegend, USA); APC lgG2bkappa isotype control, Cat. No. 400321 (Biolegend, USA); APC lgG1 kappa isotype control, Cat. No. 400121 (Biolegend, USA); APC antihuRORI, Cat. No. 130-117-942 (Miltenyi, Germany); REA Control Antibody, human IgG1 REA293, Cat. No. 130-113-446 (Miltenyi, Germany); FITC antihuHer2, Cat. No. BMS120FI (Thermo Fisher Scientific, USA); FITC antihuEGFR, Cat. No. 10001-MM08-F (Sino Biological, USA); FITC antihuEpcam, Cat. No.

### 324203 (Biolegend, USA).

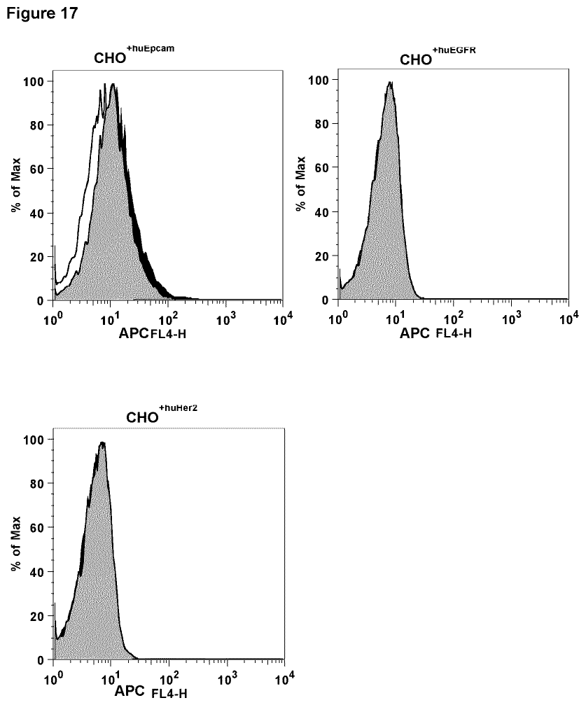

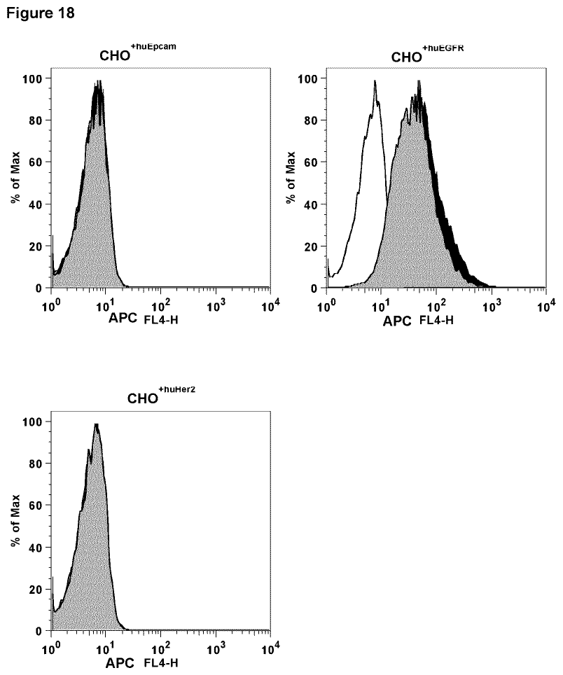

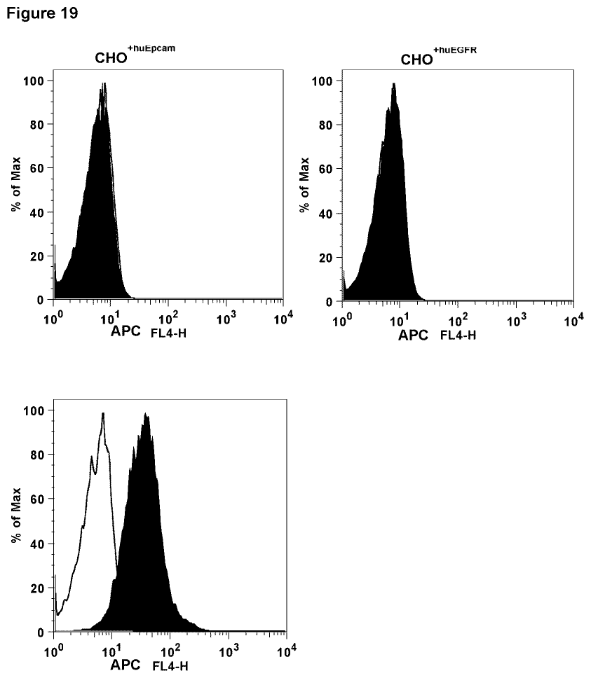

### Example 5—Target-Antigen Binding of Purified Hemibodies (Relates to FIGS. 17, 18 and 19)

Target specificity of the purified hemibodies was evaluated in flow cytometry based binding experiments using target-antigen expressing Chinese hamster ovary cells (CHO). All purified hemibodies precisely recognized their respective target and showed no aberrant binding (FIGS. 17, 18 and 19). As deduced from the FACS histograms the Epcam specific hemibodies exhibited a low target affinity, whereas binding affinities were increased in case of the Her2 and EGFR specific hemibodies.

To evaluate target binding of hemibodies single target-antigen expressing firefly luciferase (Flue) co-expressing Chinese hamster ovary cells (CHO K1, DSMZ ACC-110) were detached from culture plate using Gibco 0.05% trypsin-EDTA solution (Thermo Fisher Scientific, USA). Cells were then resuspended in FACS-buffer (PBS supplemented with 1% FBS) to a concentration of ix10*6/ml_ and incubated with hemibody construct (12.5 pg/1×10*6 cells) for 30 min. Cells were then washed, resuspended in fresh FACS-buffer and incubated for further 30 min with APC conjugated HIS tag specific antibody (Cat. No. IC050A, RDSystems, USA) at a concentration of 25 pl_antibody per 1×10*6 cells. Before cells were subjected to flow cytometry excessive antibody was removed by washing. For fluorophore signal quantification we used the FACS Calibur cytometer (BD Biosciences, USA).

### Example 6—Size Exclusion Chromatography Analysis of Purified Hemibody Constructs (Corresponds to FIGS. 21, 22 and 23)

The activity of a given antibody constructs strongly depends on its purity and aggregation status. As such, impurities and the presence of high molecular weight aggregates may adversely affect the activity and specificity of a hemibody construct. In order to assess the quality of the construct, the purified hemibodies were subjected to analytical size exclusion chromatography. As can be seen from FIGS. 21, 22 and 23, all purified hemibody constructs eluted in the monomeric form at the expected molecular size.

The analytical size exclusion chromatography was performed at a volumetric flow rate of 0.15 mL/min using the AKTApure system (Cytiva, USA) and a Superdex 200 Increase 5/150 GL size exclusion column (Cytiva, USA) with 50 mM Na-phosphate pH7.5, 300 mM NaCl solution as separation buffer. As molecular weight standard protein beta-amylase (200 kDa) and carbonic anhydrase (29 kDa) was used.

### Example 7—Elimination of Myeloma Cells In Vivo (Corresponds to FIG. 32)

KiHss Hemibodies targeting the multiple myeloma associated antigens CD38 and SLAMF7 were tested in vivo using myeloma xenografts and CD8 positive T cells isolated from human peripheral blood mononuclear cells (PBMCs) in a humanized immunodeficient NOD SCID mouse model (FIG. 32). To this end, six- to 12-week-old female NOD SCID Il2rg−/− mice were challenged intravenously i.v. with 2^{c}10*6 firefly luciferase-expressing, CD38 and SLAMF7 double-positive MM.1S cells. After 21 days and engraftment of tumor cells (day 0 of treatment), 3^{c} 10*6 CD8 positive T cells from a healthy donor were injected intravenously i.v. Mice were treated afterwards by two subcutaneous s.c. injections (nuchal fold) of 8 pg hemibodies addressing CD38 and SLAMF7 alone and in combination or 1*PBS on day 22 and day 26. To monitor the growth of luciferase-positive tumor cells, each mouse received intraperitoneally i.p. 200 pi of anesthesia cocktail (Ketavet 8 mg/ml_ and Xylavet 1.6 mg/ml_) and 200 ml luciferin (30 mg/ml_) on day 21, 28 and 35. Luciferase activity was assessed using an I VIS Lumina XR Real-Time Bioluminescence Imaging System. All animal studies received approval from the appropriate authorities (Regierung von Unterfranken, No. RUF-55.2-2531.01-79/11) and comply with all relevant ethical regulations for animal testing and research. For the in vivo model, NSG mice (stock number 5557) were purchased from the Jackson Laboratory (Bar Harbor, ME, USA) and maintained in a certified animal facility (ZEMM, Center for Experimental Molecular Medicine, Wurzburg) in accordance with European guidelines.

FIG. 32 shows that KiHss Hemibodies successfully targeted the multiple myeloma associated antigens CD38 and SLAMF7 in vivo. As can be seen in FIG. 32B, a higher survival rate was observed in mice treated with the combination of two KiHss hemibodies, forming in vivo a heterodimer on a target cell by association of their respective VH and VL, as described herein. This experiment shows that in particular such tri-specific heterodimers (formed by association of two single KiHss molecules) provide for a high specificity of T-cell engaging binding molecules of the invention when targeting a tumor cell. This improved specificity should thus also reduce off-target activity of T-cell engaging recombinant proteinaceous binding molecules of the present invention.

Example 8—Cell lines used for assaying hemibody activity. All used human tumor cell lines including MDA-MB-231 (DSMZ ACC-732), MDA-MB-453 (DSMZ ACC-65), MDA-MB-468 (DSMZ ACC-738), A549 (DSMZ ACC-107), T47D (DSMZ ACC-739), MM1.S (ATCC CRL-2974), HT29 (DSMZ ACC-299) and HCT116 (DSMZ ACC-581) were engineered to express luciferase by transduction with a replication incompetent lentiviral vector encoding firefly luciferase (Flue). To generate target-antigen and firefly luciferase (Flue) co-expressing hamster based target cells, Chinese hamster ovary cells (DSMZ ACC-110) were co-transfected with the firefly luciferase gene and the full-length coding sequences of the target antigens (huEGFR of SEQ ID NO.: 12, huHer2 of SEQ ID NO.: 13, and huEpcam of SEQ ID NO.: 14) using the PiggyBac transposon vector system (System Biosciences, USA) with PB514B-2 as the parental vector. Stable cell lines were obtained following a combined puromycin/fluorescence-activated cell sorting (FACS) based selection strategy. All human tumor cell lines were maintained in Gibco Advanced RPMI1640 medium

(Thermo Fisher Scientific, USA) supplemented with 10% Gibco HI FBS (Thermo Fisher Scientific, USA), 1:100 Gibco Glutamax (Thermo Fisher Scientific, USA) and 1:100 Gibco PSN (Thermo Fisher Scientific, USA), whereas the engineered CHO cells were grown in Gibco Ham's F-12K (Kaighn's) Medium (Thermo Fisher Scientific, USA) supplemented with 10% Gibco HI FBS (Thermo Fisher Scientific, USA), 1:100 Gibco Glutamax (Thermo Fisher Scientific, USA) and 1:100 Gibco PSN (Thermo Fisher Scientific, USA). Cells were cultured at standard atmospheric conditions (37° C., 5% C02).

### Example 9: Determination of Serum Half-Life (Corresponds to FIG. 34)

The terminal half-live (t1/2) of the KiHss hemibodies constructs was determined in separate in vivo experiments in mice fresh serum after intravenous (i.v.) injection. BALB/c mice were i.v. injected with 8 pg hemibody construct. At indicated time points, blood was taken via buccal bleed and serum was analyzed for hemibody concentration using a sandwich-ELISA assay. For detection of the 1^{st} generation hemibody, a primary capturing antibody against the His-tag and a HRP-labeled detection antibody against the FLAG-tag was used. For the 2^{nd} generation hemibody, a primary capturing antibody against the His-tag and an HRP-labeled detection antibody against the Fc-part was used. FIG. 34B shows the half-life of a KiHss hemibody pair, in particular the KiHss hemibody pair scFv antiSLAMF7 human IgGIFC hole (SEQ. ID No. 9) and VLdiL2k human IgGIFC knob (SEQ. ID No. 30) and the half-life of original hemibodies (in FIG. 34A). In this (indirect) comparison of the half-life values shown in FIG. 34A and FIG. 34B a t1/2=100 min was found for the original hemibodies and a t 1/2=500 min was found for the KiHss hemibody molecule of the invention (FIG. 34). Such a rather longer half-live is advantageous because it indicates a slow elimination from the body when used in vivo.

Unless otherwise stated, the following terms used in this document, including the description and items, have the definitions given below.

Those skilled in the art will recognize, or be able to ascertain, using not more than routine experimentation, many equivalents to the specific embodiments of the invention described herein. Such equivalents are intended to be encompassed by the present invention.

It is to be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

1. A recombinant proteinaceous binding molecule comprising:
a) a first binding moiety, capable of binding an antigen, having a first binding site for a first antigen,
b) a variable domain of either an antibody light chain or an antibody heavy chain of a second binding site for a second antigen, and
c) an Fc fragment comprising a first and a second heavy chain,
wherein the first and the second heavy chain each comprise one immunoglobulin CH2 domain and one immunoglobulin CH3 domain, and wherein the CH3 domain of the first heavy chain and the CH3 domain of the second heavy chain meet each other at an interface, which interface comprises an original interface between the CH3 domains, wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
wherein the CH3 domain of the other heavy chain is altered so that within the original interface of the second CH3 domain thereof meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and wherein the variable domain of either the antibody light chain or the antibody heavy chain and the first binding moiety are linked via the Fc fragment.

2. A heterodimeric recombinant proteinaceous binding molecule comprising a heterodimer of recombinant proteinaceous molecules comprising monomers,
wherein a first monomer consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of a second heavy chain meet each other at an interface, which interface comprises an original interface between the CH3 domains wherein the CH3 domain of the first or second heavy chain is altered, that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
wherein the CH3 domain of the other heavy chain is altered so that within the original interface of the second CH3 domain thereof meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and wherein the variable domain of either the antibody light chain or the antibody heavy chain and the first binding moiety are linked via the Fc fragment,
wherein the second monomer consists of a binding moiety having a first binding site for a first antigen; a variable domain of either an antibody light chain or an antibody heavy chain of a second binding site for a second antigen; and an Fc fragment comprises a first and a second heavy chain wherein the first and the second heavy chain each comprise one immunoglobulin CH2 domain and one immunoglobulin CH3 domain, and wherein the CH3 domain of the first heavy chain and the CH3 domain of the second heavy chain each meet each other at an interface,
wherein the CH3 domain of the first or second heavy chain is altered, so that within the original interface of the CH3 domain of one heavy chain that meets the original interface of the CH3 domain of the other heavy chain, an amino acid residue is replaced with an amino acid residue having a larger side chain volume, thereby generating a protuberance within the interface of the CH3 domain of the one heavy chain which is positionable in a cavity within the interface of the CH3 domain of the other heavy chain, and
wherein the CH3 domain of the other heavy chain is altered so that within the original interface of the second CH3 domain of that meets the interface of the first CH3 domain of the other heavy chain, an amino acid residue is replaced with an amino acid residue having a smaller side chain volume, thereby generating a cavity within the interface of the second CH3 domain within which the protuberance within the interface of the first CH3 domain is positionable, and
wherein the variable domain of either the antibody light chain or the antibody heavy chain and the first binding moiety are linked via the Fc fragment,
wherein the first antigen of the first monomer and the first antigen of the second monomer are two antigens of different identity,
wherein the variable domain of an antibody light chain of the second binding site of the first monomer and the variable domain of an antibody heavy chain of the second binding site of the second monomer associate thereby forming the second binding site and dimerizing the heterodimer.

3. The recombinant proteinaceous binding molecule of claim 1, wherein the binding moiety having a first binding site is an antibody fragment.

4. The recombinant proteinaceous binding molecule of claim 3, wherein the antibody fragment is selected from the group consisting of a divalent antibody fragment, and a monovalent antibody fragment, wherein the divalent antibody fragment is optionally an F(ab′)2-fragment, or a divalent single-chain Fv fragment, wherein the monovalent antibody fragment is selected from the group consisting of a binding moiety, a Fv fragment, a single-chain Fv fragment (scFv) and a camelid single domain antibody.

5. The recombinant proteinaceous binding molecule of claim 1, wherein the binding moiety having a first binding site is a binding molecule with antibody-like binding properties.

6. The recombinant proteinaceous binding molecule of claim 5 wherein the binding molecule with antibody-like binding properties is selected from the group consisting of: an aptamer, an affilin, an affibody, an affimer, an atrimer, an anticalin, an adnectin, an avimer, an alphabody, an autofluorescent protein, a centyrin, a DARPin, a fynomer, a glubody, a kappabody, a Kringle domain, a Kunitz domain, a knottin, a nanofitin, a repebody, an antigen specific t-cell receptor, any receptor-protein, any receptor-protein ligand.

7. The recombinant proteinaceous binding molecule of claim 1 wherein the immunoglobulin CH3 domain of the first or the second heavy chain comprises at least one of the amino acid substitutions T366S, L368A and Y407V (CH3 hole-chain) and the immunoglobulin CH3 domain of the second heavy chain comprises the amino acid substitution T366W (CH3 knob-chain), wherein the CH3 hole-chain further comprises the amino acid substitution Y349C and the CH3-knob chain further comprises the amino acid substitution S354C.

8. The recombinant proteinaceous binding molecule of claim 1, wherein at least one amino acid residue of the CH2 domain of the recombinant proteinaceous binding molecule that is able to mediate binding to Fc receptors is lacking or mutated, wherein the amino acid residues are selected from the group consisting of sequence position 230, 231, 232, 233, 234, 235, 236, 237, 238, 265, 297, 327, and 330.

9. The recombinant proteinaceous binding molecule of claim 8, wherein the least one mutation is selected from the group consisting of a substitution Leu234->Ala, a substitution Leu235->Ala, a substitution Asn297->Ala, and a substitution Pro329->Ala.

10. The recombinant proteinaceous binding molecule of claim 1, wherein the first binding site of the recombinant proteinaceous binding molecule binds a tumor-associated antigen.

11. The recombinant proteinaceous binding molecule of claim 1, wherein the light chain of the variable fragment of the second binding site fused to the CH3-hole chain may have sequence identity of at least 80% to the sequence shown in SEQ ID NO.:2, or wherein the light chain of the variable fragment of the second binding site fused to the CH3-knob chain may have sequence identity of at least 80% to the sequence shown in SEQ ID NO.:3, or wherein the heavy chain of the variable fragment of the second binding site fused to the CH3-hole chain may have sequence identity of at least 80% to the sequence selected from the sequences shown in SEQ ID NO.:1 or SEQ ID NO.:10.

12. The recombinant proteinaceous binding molecule of claim 1, wherein the binding moiety having a first binding site fused to the CH3-knob chain has a sequence identity of at least 80% to the sequence shown in SEQ ID NO.:4, or wherein the binding moiety having a first binding site fused to the CH3-knob chain has a sequence identity of at least 80% to the sequence shown in SEQ ID NO.: 5, or wherein the binding moiety having a first binding site fused to the CH3-knob chain has a sequence identity of at least 80% to the sequence shown in SEQ ID NO.:6, or wherein the binding moiety having a first binding site fused to the CH3-hole chain has a sequence identity of at least 80% to the sequence shown in SEQ ID NO.:9, or wherein the binding moiety having a first binding site fused to the CH3-knob chain has a sequence identity of at least 80% to the sequence shown in SEQ ID NO.: 11.

13. The recombinant proteinaceous binding molecule of claim 1, wherein the light chain or the heavy chain of a second binding site for a second antigen are a light chain or a heavy chain of a second binding site for a T-cell, NK (natural killer), Monocyte, Macrophage, Dendritic Cell or Neutrophilic Granulocyte cell specific receptor molecule (CD3, the T cell receptor (TCR), CD28, CD16, NKG2D, 0x40, 4-1 BB, CD2, CD4, CD5, CD8, CD95, CD32a, CD40, CD89, CD64, NKp30, NKp40, PD1, CTLA4, LFA1.

14. A pharmaceutical composition comprising a recombinant proteinaceous binding molecule as defined in claim 1.

15. The recombinant proteinaceous binding molecule of claim 1 as a treatment or for diagnosis of a disease, wherein the disease is optionally a proliferative disease.

16. The recombinant proteinaceous binding molecule molecule of claim 15, wherein the proliferative disease is selected from the group consisting of hematopoietic malignancies, optionally acute and chronic myeloic and lymphatic leukemias lymphomas, solid tumors optionally tumors of the gastrointestinal tract, lung, kidney, prostate, breast, brain, ovary, uterus, mesenchymal tumors and melanoma.

17. A nucleic acid molecule encoding a recombinant proteinaceous binding molecule as defined in claim 1.

18. A nucleic acid molecule of claim 17 comprised in a vector.

19. A host cell comprising a nucleic acid molecule of claim 17.

20. A method of producing a recombinant proteinaceous binding molecule of claim 1, comprising expressing a nucleic acid encoding the recombinant proteinaceous binding molecule or the monomer of the heterodimeric recombinant proteinaceous binding molecule under conditions allowing expression of the nucleic acid.

21. The recombinant proteinaceous binding molecule of claim 15 as treatment of a disease, wherein the recombinant proteinaceous binding molecule forms a heterodimer only in vivo on a target cell, thereby reducing “off target activation”.


SEQUENCE LISTING (51 sequences)

SEQ ID NO: 1 - protein, 376 aa, Artificial Sequence
  VHUCHT1 human IgG1 Fc hole
METDTLLVFVLLVWVPAGNGEFEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 2 - protein, 362 aa, Artificial Sequence
  VLUCHT1 human IgG1 Fc hole
METDTLLVFVLLVWVPAGNGEFDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 370 aa, Artificial Sequence
  VLUCHT1 human IgG1Fc knob
METDTLLVFVLLVWVPAGNGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKVDGSHHHHHH

SEQ ID NO: 4 - protein, 425 aa, Artificial Sequence
  Darpin antiEpcam Ec4 human IgG1 Fc knob
METDTLLVFVLLVWVPAGNGEFDLGKKLLEAARAGQDDEVRILMANGADVNAYDFGTTPLHLAATHGHLEIVEVLLKDGADVNAQDDWGITPLHLAAYNGHLEIVEVLLKYDADVNAHDTRGWTPLHLAAINGHLEIVEVLLKNGADVNAQDKFGKTPFDLAIDNGNEDIAEVLQKAAKLNGSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKVDGSHHHHHH

SEQ ID NO: 5 - protein, 391 aa, Artificial Sequence
  VHH antiEGFR 9G8 human IgG1 Fc knob
METDTLLVFVLLVWVPAGNGEFEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKVDGSHHHHHH

SEQ ID NO: 6 - protein, 507 aa, Artificial Sequence
  scFv antiHer2 Trastuzumab human IgG1 Fc knob
METDTLLVFVLLVWVPAGNGEFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKVDGSHHHHHH

SEQ ID NO: 7 - protein, 233 aa, artificial sequence
  human IgG1 Fc hole
SGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 243 aa, artificial sequence
  human IgG1 Fc knob
SGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKVDGSHHHHHH

SEQ ID NO: 9 - protein, 494 aa, artificial sequence
  scFv antiSLAMF7 human IgG1FC hole
METDTLLVFVLLVWVPAGNGEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGGGGSGGGGSGGGGSDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 373 aa, artificial sequence
  VHUCHT1 human IgG1FC hole
METDTLLVFVLLVWVPAGNGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 508 aa, artificial sequence
  scFv antiCD38 human IgG1FC knob
METDTLLVFVLLVWVPAGNGQVQLVESGGGLVQPGGSLRLSCAASGFTFSSYGMHWVRQAPGKGLEWVSNIYSDGSNTFYADSVKGRFTISRDNSKNTLYLQMNSLRAEDTAVYYCARNMYRWPFHYFFDYWGQGTLVTVSSGGGGSGGGGSGGGGSDIELTQPPSVSVAPGQTARISCSGDNIGNKYVSWYQQKPGQAPVVVIYGDNNRPSGIPERFSGSNSGNTATLTISGTQAEDEADYYCSSYDSSYFVFGGGTKLTVLGQSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKVDGSHHHHHH

SEQ ID NO: 12 - protein, 1210 aa, Homo sapiens [sequence omitted]

SEQ ID NO: 13 - protein, 1255 aa, Homo Sapiens [sequence omitted]

SEQ ID NO: 14 - protein, 314 aa, Homo Sapiens
  huEpcam (1)..(314): huEpcam
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 15 - DNA, 1134 bp, Artificial Sequence
  VHUCHT1 human IgG1FC hole conding sequence
atggaaaccgacacactgctggtgttcgtgctgctcgtgtgggtgccagctggaaacggcgaattcgaagtgcagctggttgaatcaggtggtggtctggttcagccaggtggtagcctgcgtctgagctgtgcagcaagtggttattcttttaccggttataccatgaattgggtgcgccaagctccgggtaaaggcctggaatgggttgcactgattaatccgtataaaggtgtgagcacctataatcagaaattcaaagatcgctttaccatcagcgtggataaaagcaaaaataccgcctacctgcaaatgaatagtctgcgtgcagaggatacagcagtgtattattgtgcacgtagcggttattatggtgatagcgattggtatttcgacgtgtggggacaaggtacactggtgaccgtgagctccggaggtggcggctccgacaagacccatacttgtcctccatgtcctgctccagaggctgctggtggcccttccgttttcctgtttccacctaagcctaaggacaccctgatgatctctcggacccctgaagtgacctgcgtggtggtcgatgtgtctcacgaggacccagaagtgaagttcaattggtacgtggacggcgtggaagtgcacaatgccaagaccaagcctagagaggaacagtacgcctccacctacagagtggtgtctgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccttgcctcctagccgggacgagctgaccaagaatcaggtgtccctgtcctgtgccgtgaagggcttctacccttccgatatcgctgtggaatgggagagcaacggccagccagagaacaactacaagaccacacctcctgtgctggacagcgacggctcattcttcctggtgtccaagctgacagtggacaagtccagatggcagcagggcaacgtgttctcctgctccgtgatgcacgaggccctgcacaatcactacacacagaagtccctgagcctgtctccaggcaagtgatga

SEQ ID NO: 16 - DNA, 1092 bp, artificial sequence
  VLUCHT1 human IgG1FC hole coding sequence
atggaaaccgacacactgctggtgttcgtgctgctcgtgtgggtgccagctggaaacggcgaattcgatattcagatgacacagagcccgagcagtctgagcgcaagcgttggtgatcgtgtgaccattacctgtcgtgcaagccaggatattcgtaattatctgaactggtatcaacaaaaaccaggcaaagcaccgaaactgctgatttattacaccagccgtctggaaagcggtgtgccgagccgttttagtggtagcggttcaggtacagattatacactgaccattagcagcctgcagccggaagattttgcaacctattactgtcagcagggcaataccctgccttggacatttggccagggcacgaaagttgaaattaaatccggaggtggcggctccgacaagacccatacttgtcctccatgtcctgctccagaggctgctggtggcccttccgttttcctgtttccacctaagcctaaggacaccctgatgatctctcggacccctgaagtgacctgcgtggtggtcgatgtgtctcacgaggacccagaagtgaagttcaattggtacgtggacggcgtggaagtgcacaatgccaagaccaagcctagagaggaacagtacgcctccacctacagagtggtgtctgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccttgcctcctagccgggacgagctgaccaagaatcaggtgtccctgtcctgtgccgtgaagggcttctacccttccgatatcgctgtggaatgggagagcaacggccagccagagaacaactacaagaccacacctcctgtgctggacagcgacggctcattcttcctggtgtccaagctgacagtggacaagtccagatggcagcagggcaacgtgttctcctgctccgtgatgcacgaggccctgcacaatcactacacacagaagtccctgagcctgtctccaggcaagtgatga

SEQ ID NO: 17 - DNA, 1116 bp, artificial sequence
  VLUCHT1 human IgG1FC knob coding sequence
atggaaacagataccctgctggtgttcgtgctgctcgtgtgggtgccagctggaaatggcgatattcagatgacacagagcccgagcagtctgagcgcaagcgttggtgatcgtgtgaccattacctgtcgtgcaagccaggatattcgtaattatctgaactggtatcaacaaaaaccaggcaaagcaccgaaactgctgatttattacaccagccgtctggaaagcggtgtgccgagccgttttagtggtagcggttcaggtacagattatacactgaccattagcagcctgcagccggaagattttgcaacctattactgtcagcagggcaataccctgccttggacatttggccagggcacgaaagttgaaattaaatccggaggcggcggaagcgacaagacccatacttgtcctccatgtcctgctccagaggctgctggtggcccttctgtgttcctgtttcctccaaagcctaaggacaccctgatgatctctcggacccctgaagtgacctgcgtggtggtcgatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtttccaacaaggccctgcctgctcctatcgaaaagaccatctccaaggccaagggccagcctagggaaccccaggtttacaccttgcctccatgccgggatgagctgaccaagaaccaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgactgtggacaagtccagatggcagcagggcaacgtgttctcctgctccgtgatgcacgaggccctgcacaatcactacacacagaagtccctgtctctgagccccggaaaggtcgacggctctcaccaccaccatcaccactgatga

SEQ ID NO: 18 - DNA, 1281 bp, artificial sequence
  Darpin antiEpcam Ec4 human IgG1FC knob coding sequence
atggaaaccgacacactgctggtgttcgtgctgctcgtgtgggtgccagctggaaacggcgaattcgatctgggtaaaaaactgctggaagcagcacgtgcaggtcaggatgatgaagttcgtattctgatggcaaatggtgcagatgttaacgcctatgattttggcaccacaccgctgcatctggcagcaacacatggtcatctggaaattgttgaagttctgctgaaagatggtgccgatgtgaatgcacaggatgattggggtattacccctctgcacctggctgcatataacggccacctggaaatcgtagaggtgctgctgaaatatgatgcagacgttaatgcgcatgatacccgtggttggaccccactgcacctggcagcaattaatggacatctggaaatcgtggaagtgctgctgaaaaatggcgctgatgttaacgcgcaggataaatttggtaaaaccccgtttgatctggccattgataatggcaatgaagatattgcagaggtcctgcaaaaagcagcaaaactgaatggctccggaggcggcggaagcgacaagacccatacttgtcctccatgtcctgctccagaggctgctggtggcccttctgtgttcctgtttcctccaaagcctaaggacaccctgatgatctctcggacccctgaagtgacctgcgtggtggtcgatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtttccaacaaggccctgcctgctcctatcgaaaagaccatctccaaggccaagggccagcctagggaaccccaggtttacaccttgcctccatgccgggatgagctgaccaagaaccaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgactgtggacaagtccagatggcagcagggcaacgtgttctcctgctccgtgatgcacgaggccctgcacaatcactacacacagaagtccctgtctctgagccccggaaaggtcgacggctctcaccaccaccatcaccactgatga

SEQ ID NO: 19 - DNA, 1179 bp, Artificial sequence
  VHH antiEGFR 9G8 human IgG1FC knob  coding sequence
atggaaaccgacacactgctggtgttcgtgctgctcgtgtgggtgccagctggaaacggcgaattcgaagttcagctggttgaaagcggtggtggtctggttcaggcaggcggtagcctgcgtctgagctgtgcagcaagcggtcgtacctttagcagctatgcaatgggttggtttcgtcaggcaccgggtaaagaacgtgaatttgttgttgccattaattggagcagcggtagcacctattatgcagatagcgttaaaggtcgttttaccattagccgtgataatgccaaaaacaccatgtacctgcaaatgaatagcctgaaaccggaagataccgcagtgtattattgtgcagcaggctatcagattaatagcggcaactacaacttcaaggattacgagtatgattattggggtcagggcacccaggttaccgttagctccggaggcggcggaagcgacaagacccatacttgtcctccatgtcctgctccagaggctgctggtggcccttctgtgttcctgtttcctccaaagcctaaggacaccctgatgatctctcggacccctgaagtgacctgcgtggtggtcgatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtttccaacaaggccctgcctgctcctatcgaaaagaccatctccaaggccaagggccagcctagggaaccccaggtttacaccttgcctccatgccgggatgagctgaccaagaaccaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgactgtggacaagtccagatggcagcagggcaacgtgttctcctgctccgtgatgcacgaggccctgcacaatcactacacacagaagtccctgtctctgagccccggaaaggtcgacggctctcaccaccaccatcaccactgatga

SEQ ID NO: 20 - DNA, 1527 bp, artificial sequence
  scFv antiHer2 Trastuzumab human IgG1FC knob coding sequence
atggaaaccgacacactgctggtgttcgtgctgctcgtgtgggtgccagctggaaacggcgaattcgaagtccagctggttgaaagtggcggtggcctggtgcaaccgggtggctctctgcgtctgagttgtgcggcctccggcttcaacattaaagatacctatatccactgggtccgtcaggcaccgggtaaaggcctggaatgggtggctcgcatttacccgaccaatggttatacgcgttacgcagatagcgtcaaaggccgcttcacgatctcagcagacacctcgaagaacacggcttatctgcagatgaatagtctgcgtgcagaagataccgctgtttattactgctcccgctggggtggcgatggtttttatgcgatggactactggggtcaaggcaccctggtgacggtttctagtggtggcggtggctcgggtggcggtggcagcggtggcggtggctctgatattcagatgacccaaagcccgtcctcactgtctgcaagtgttggcgatcgtgtcaccatcacgtgtcgcgcttctcaggacgtgaataccgcagttgcttggtatcagcaaaagccgggtaaagcgccgaagctgctgatttattccgcctcatttctgtacagcggcgttccgtctcgtttctcgggtagccgctctggcaccgattttaccctgacgatctcgagcctgcagccggaagactttgccacctattactgccagcaacactatacgacgccgccgacgtttggtcaaggcacgaaagtggaaatcaagtccggaggcggcggaagcgacaagacccatacttgtcctccatgtcctgctccagaggctgctggtggcccttctgtgttcctgtttcctccaaagcctaaggacaccctgatgatctctcggacccctgaagtgacctgcgtggtggtcgatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtttccaacaaggccctgcctgctcctatcgaaaagaccatctccaaggccaagggccagcctagggaaccccaggtttacaccttgcctccatgccgggatgagctgaccaagaaccaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgactgtggacaagtccagatggcagcagggcaacgtgttctcctgctccgtgatgcacgaggccctgcacaatcactacacacagaagtccctgtctctgagccccggaaaggtcgacggctctcaccaccaccatcaccactgatga

SEQ ID NO: 21 - DNA, 705 bp, artificial sequence
  human IgG1FC hole coding sequence
tccggaggtggcggctccgacaagacccatacttgtcctccatgtcctgctccagaggctgctggtggcccttccgttttcctgtttccacctaagcctaaggacaccctgatgatctctcggacccctgaagtgacctgcgtggtggtcgatgtgtctcacgaggacccagaagtgaagttcaattggtacgtggacggcgtggaagtgcacaatgccaagaccaagcctagagaggaacagtacgcctccacctacagagtggtgtctgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccttgcctcctagccgggacgagctgaccaagaatcaggtgtccctgtcctgtgccgtgaagggcttctacccttccgatatcgctgtggaatgggagagcaacggccagccagagaacaactacaagaccacacctcctgtgctggacagcgacggctcattcttcctggtgtccaagctgacagtggacaagtccagatggcagcagggcaacgtgttctcctgctccgtgatgcacgaggccctgcacaatcactacacacagaagtccctgagcctgtctccaggcaagtgatga

SEQ ID NO: 22 - DNA, 735 bp, artificial sequence
  human IgG1FC knob coding sequence
tccggaggcggcggaagcgacaagacccatacttgtcctccatgtcctgctccagaggctgctggtggcccttctgtgttcctgtttcctccaaagcctaaggacaccctgatgatctctcggacccctgaagtgacctgcgtggtggtcgatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtttccaacaaggccctgcctgctcctatcgaaaagaccatctccaaggccaagggccagcctagggaaccccaggtttacaccttgcctccatgccgggatgagctgaccaagaaccaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgactgtggacaagtccagatggcagcagggcaacgtgttctcctgctccgtgatgcacgaggccctgcacaatcactacacacagaagtccctgtctctgagccccggaaaggtcgacggctctcaccaccaccatcaccactgatga

SEQ ID NO: 23 - DNA, 3636 bp, Homo Sapiens
  human EGFR coding sequence (1)..(3636): human EGFR coding sequence
atgaggccatctggaacagctggtgctgctctgctggctctgttggctgctctgtgtccagctagtagagccctggaagagaagaaagtgtgccagggcacctccaacaagctgacacagctgggaaccttcgaggaccacttcctgtctctccagcggatgttcaacaactgcgaggtggtgctgggcaacctggaaatcacctacgtgcagcggaactacgacctgtccttcctgaaaaccatccaagaggtggccggctacgtgctgatcgccctgaataccgtggaaagaatccctctggaaaacctccagatcatccggggcaacatgtactacgagaacagctacgccctggccgtgctgtctaactacgacgctaacaagaccggcctgaaagaactgcccatgcggaacctgcaagagatcctgcatggcgccgtgcggttcagcaacaacccagctctgtgcaacgtggaatccatccagtggcgggatattgtgtcctccgacttcctgagcaacatgagcatggacttccagaaccacctgggctcttgccagaagtgcgacccttcttgccctaacggctcttgttggggagccggcgaggaaaactgtcagaagctgaccaagatcatctgcgcccagcagtgctccggcagatgcagaggaaagtccccttctgactgctgccacaaccagtgtgccgctggctgtacaggccctcgcgaatctgattgcctcgtgtgccggaagttccgcgacgaggctacctgcaaggatacttgccctcctctgatgctgtacaaccccaccacctaccagatggacgtgaaccccgagggaaagtactccttcggcgccacctgtgtgaagaagtgccccagaaactacgtcgtgaccgaccacggctcctgcgttagagcttgtggcgctgactcctacgagatggaagaggatggcgtgcggaagtgcaagaaatgcgagggcccttgcagaaaagtgtgcaacggcatcggcatcggagagttcaaggactccctgtccatcaacgccaccaacatcaagcacttcaagaactgcacctccatctccggcgacctgcacattctgcctgtggccttcagaggcgactccttcacacatacccctccactggaccctcaagagctggacatcctcaagaccgtgaaagagatcaccggctttctgctgatccaggcttggcctgagaaccggaccgatctgcacgccttcgagaatttggagatcatcagaggccggaccaagcagcatggccagttttctctggctgtggtgtccctgaacatcaccagcctgggcctgagatctctgaaagaaatctccgacggcgacgtgatcatcagcggcaacaagaacctgtgctacgccaacaccatcaactggaagaagctgttcggcacctccggccagaaaacaaagatcatttccaaccggggcgagaactcctgcaaggctaccggccaagtctgtcacgccctgtgttctccagaaggctgttggggacctgagcctagagactgtgtgtcctgtcggaacgtgtcccggggcagagaatgcgtggacaagtgcaatctgctggaaggcgagccccgcgagttcgtggaaaactctgagtgcatccagtgtcaccccgagtgtctgcctcaggccatgaatatcacctgtaccggcagaggccccgacaactgtatccagtgcgcccactatatcgacggccctcactgcgtgaaaacctgtcctgctggcgtgatgggagagaacaacaccctcgtgtggaagtacgccgatgctggccatgtgtgccatctgtgtcaccctaactgtacctacggctgcaccggacctggcctggaaggctgtcctacaaacggccctaagatcccctctatcgccaccggaatggttggagccctgctgctgttgctggtggtggctcttggcatcggcctgttcatgcggagaaggcacattgtgcggaagcggaccctgcggagactgctgcaagaaagagagctggtcgagcccctgacaccttctggcgaagctcctaatcaggccctgctgagaatcctcaaagagacagagttcaaaaagatcaaggtgctcggctccggcgccttcggcacagtttacaaaggcctgtggattcccgagggcgagaaagtgaagatccccgtggccatcaaagagctgagagaggccacatctcccaaggccaacaaagagattctggacgaggcctacgtgatggcctccgtggataatccccacgtgtgcagactgctgggaatctgcctgacctctaccgtgcagctgatcacccagctgatgcctttcggctgcctgctggattacgtgcgcgagcacaaggacaacatcggctcccagtacctgctgaattggtgcgtccagatcgccaagggcatgaactacctggaagatcggcggctggtgcacagagatctggccgctagaaacgtgctggtcaagacccctcagcacgtgaagatcaccgatttcggcctggctaagctgctgggagctgaggaaaaagagtaccacgctgaaggcggcaaggtgcccatcaagtggatggccctggaatctatcctgcatcggatctacacccaccagtccgacgtgtggtcttacggcgttacagtgtgggaactgatgaccttcggctccaagccttacgacggcatccctgcctccgagatctcctccatcctggaaaagggcgagagactgccccagcctcctatctgcaccatcgacgtgtacatgattatggtcaagtgctggatgatcgacgccgacagccggcctaagttcagagagctgatcatcgagttctccaagatggccagagatccccagagatacctggtcatccagggcgacgagcggatgcatcttccttctccaaccgactccaacttctacagagccctgatggacgaagaggacatggacgacgtggtggacgccgacgagtatctgatccctcagcaaggcttcttcagctccccttccacctctcggactcctctgctgtcctctctgtccgccacctctaacaattccaccgtggcctgcatcgaccggaacggcttgcagagctgccctatcaaagaggatagcttcctccagcggtacagcagcgatcctacaggcgctctgaccgaggactctatcgacgacaccttcctgcctgtgcctgagtacatcaaccagtccgtgcctaagaggcctgctggcagcgtgcagaatcccgtgtaccacaatcagccactgaaccccgctcctagcagggaccctcattatcaggaccctcactctaccgccgtgggcaaccctgagtacctgaacacagtgcagcctacctgcgtgaactccaccttcgatagccctgctcactgggctcagaagggctctcatcagatctccctggacaaccccgactaccagcaggatttcttcccaaaagaggctaagccaaacggcatcttcaagggctccaccgccgagaatgccgaatacctgagagtggcccctcagtcctctgagttcatcggcgcttgatga

SEQ ID NO: 24 - DNA, 3771 bp, Homo Sapiens
  human Her2 coding sequence (1)..(3771): human Her2 coding sequence
atggaactggctgctctgtgtagatggggcctgctgctggctttgttgcctcctggtgctgcttctacccaagtgtgcaccggcaccgacatgaagctgagactgcctgcctctcctgagacacacctggacatgctgagacacctgtaccagggctgtcaggtggtgcagggcaatctggaactgacctacctgcctaccaacgccagcctgagcttcctccaggacatccaagaggtgcagggatacgtgctgatcgcccacaatcaagtgcgccaggtgccactccagcggctgagaatcgttagaggcacccagctgttcgaggacaactacgctctggccgtgctggacaatggcgaccctctgaacaacaccacacctgtgacaggcgcttctcctggcggactgagagagttgcagctgagatccctgaccgagatcctgaaaggcggagtgctgatccagcggaaccctcagctgtgctaccaggacaccatcctgtggaaggacatcttccacaagaacaaccagctggctctgaccctgatcgacaccaaccggtccagagcttgtcacccctgttctcccatgtgcaagggctccagatgttggggcgagtcctccgaggattgccagtctctgaccagaaccgtgtgtgctggcggctgtgctagatgcaaaggacctctgcctaccgactgctgccacgagcaatgtgccgctggatgtaccggacctaagcactctgactgcctggcctgcctgcacttcaaccactctggaatctgcgagctgcactgccctgctctggtcacctacaacaccgataccttcgagtccatgcctaatcctgagggcagatataccttcggcgcctcctgtgtgaccgcctgtccttacaactacctgtctaccgacgtgggcagctgtaccctcgtgtgtcccctgcataatcaagaagtgaccgccgaggacggcacccagagatgcgagaagtgctctaagccttgcgccagagtgtgttacggcctcggcatggaacacctgagagaagtgcgggctgtgacctccgccaatatccaagagtttgccggctgcaagaagatctttggctccctggcctttctgcccgagtctttcgatggcgatcccgcctctaacaccgcacctcttcagcctgaacagctgcaagtgttcgagacactggaagagatcaccggctacctgtacatctccgcctggccagactctctgcctgacctgtctgtgttccagaacctgcaagtgatccggggcagaatcctgcacaacggcgcttactccctgacactgcaaggcctgggaatctcttggctgggcctgagatctctgagagaactcggctctggactggccctgatccaccacaatacccacctgtgcttcgtgcacaccgtgccttgggaccagctgtttagaaaccctcaccaggctctgctgcacaccgccaacagacctgaggatgagtgtgtcggcgaaggcctggcttgccatcagctgtgtgctagaggacactgttggggccctggacctacacagtgcgtgaactgctcccagttcctgagaggccaagagtgcgtggaagagtgcagagtgttgcagggcctgcctcgcgagtacgtgaacgctagacactgtctgccctgccatcctgagtgccagcctcagaatggctccgtgacctgttttggccctgaggccgatcagtgtgtggcctgcgctcactataaggaccctccattctgcgtggcccggtgtccttctggcgtgaagcctgatctgtcctacatgcccatctggaagttccccgatgaggaaggcgcttgccagccttgtcctatcaactgcacccacagctgcgtggacctggacgataagggctgtcctgctgagcagagagcctctcctctgacctccatcatctctgccgtcgtgggaatcctgctggtggtggtgctgggagtcgtgttcggcatcctgatcaagcggcggcagcagaagatccggaagtacaccatgcggcggctgctgcaagagacagagctggtggaacctctgacacccagcggcgctatgccaaaccaggctcagatgcggattctgaaagaaaccgagctgcggaaagtgaaggtgctcggcagcggagcttttggcaccgtgtacaaaggcatctggattcccgacggcgagaacgtgaagatccccgtggctatcaaggtgctgagagagaacacctctcctaaggccaacaaagagatcctggacgaggcctacgtgatggctggtgtcggctccccttatgtgtccagactgctgggcatctgtctgacctctaccgtgcagctggtcactcagctgatgccttacggctgcctgctggatcacgtgcgcgagaatagaggcagactgggctctcaggacctgctgaactggtgcatgcagatcgccaagggcatgtcctacctcgaggatgtgcggctggtccacagagatctggctgccagaaacgtgctggtcaagtcccctaaccatgtgaagatcaccgacttcggcctggctaggctgctggacatcgacgagacagagtaccatgccgatggcggcaaggtgcccatcaagtggatggccctggaatccatcctgcggcggagattcacccaccagtctgacgtgtggtcttacggcgtgacagtgtgggagctgatgaccttcggagccaagccttacgacggcatccctgccagagagatccccgacctgctggaaaagggcgagagattgcctcagcctcctatctgcaccatcgacgtgtacatgattatggtcaagtgctggatgatcgactccgagtgccggcctagattccgcgaactggtgtctgagttctcccggatggccagagatcctcagagattcgtggtcatccagaacgaggatctgggccctgcttctcctctggactccaccttctacagatccctgctcgaggacgacgacatgggcgatctggtggacgccgaggaatatctggtgcctcagcagggcttcttctgtcccgatcctgctcctggcgctggcggaatggtgcatcacagacaccggtctagctccaccagaagtggtggcggagatctgacactgggcctcgagccatctgaagaggaagcccctagaagccctctggctccttctgaaggcgccggatctgatgtgttcgacggcgatcttggcatgggcgctgctaagggacttcagtccctgcctacacacgacccatctccactccagagatacagcgaggacccaaccgtgcctctgccttctgagacagacggctatgtggccccactgacatgtagccctcagcctgagtatgtgaaccagccagatgtccggcctcagccaccttctccaagagaaggacctttgccagccgctagaccagccggtgctactctggaaagacccaagacactgagccccggcaagaacggcgttgtgaaggacgtgttcgctttcggcggagccgtggaaaaccccgagtatctgacacctcaaggcggagctgcaccccagcctcatccacctccagcattctctcccgccttcgacaacctgtactactgggaccaagatcctcctgagagaggcgcccctccatctacctttaagggcacccctaccgccgagaatcctgagtaccttggcctggatgtgcccgtgtgatga

SEQ ID NO: 25 - DNA, 945 bp, Homo Sapiens
  human Epcam coding sequence (1)..(945): human Epcam coding sequence
atggcccctccacaggtgctggcctttggactgctgctggccgctgccaccgccacatttgctgccgctcaggaagagtgcgtgtgcgagaactacaagctggccgtgaactgcttcgtgaacaacaaccggcagtgccagtgcaccagcgtgggagcccagaacaccgtgatctgcagcaaactggccgccaagtgcctcgtgatgaaggccgagatgaacggcagcaagctgggcagaagggccaagcctgaaggcgccctgcagaacaacgacggcctgtacgaccccgactgtgacgagagcggcctgttcaaggccaagcagtgcaacggcaccagcatgtgttggtgcgtgaacactgccggcgtgcggagaaccgacaaggacaccgagatcacctgtagcgagagagtgcggacctactggatcatcatcgagctgaagcacaaggccagagagaagccctacgacagcaagagcctgcggaccgctctgcagaaagagatcaccaccagataccagctggaccccaagttcatcaccagcatcctgtacgagaacaatgtgatcaccatcgacctggtgcagaacagcagccagaaaacccagaacgacgtggacattgccgacgtggcctactacttcgagaaggacgtgaagggcgagagcctgttccacagcaagaaaatggacctgaccgtgaatggcgagcagctggacctggatcctggccagaccctgatctactacgtggacgagaaggcccccgagttcagcatgcagggactgaaggctggcgtgatcgccgtgatcgtggtggtcgtgattgccgtggtggccggcattgtggtgctcgtgatcagccggaagaaacggatggccaaatacgagaaagccgagatcaaagaaatgggcgagatgcacagagagctgaacgcctga

SEQ ID NO: 26 - DNA, 1488 bp, Artificial sequence
  scFv antiSLAMF7 human IgG1FC hole coding sequence
atggaaacagataccctgctggtgttcgtgctgctcgtgtgggtgccagctggaaatggcgaagtgcagctggtggaatctggcggaggattggttcagcctggcggctctctgagactgtcttgtgccgcctctggcttcgacttctcccggtactggatgtcctgggtccgacaggctcctggcaaaggcctggaatggatcggcgagatcaaccccgactcctccaccatcaactacgcccctagcctgaaggacaagttcatcatctcccgggacaacgccaagaactccctgtacctccagatgaacagcctgagagccgaggacaccgccgtgtactactgtgctagacccgacggcaactactggtacttcgatgtgtggggccagggcaccctggtcacagtttctagcggcggaggtggaagcggaggcggaggtagtggtggtggcggatctgacatccagatgacccagtctccatccagcctgtctgcctctgtgggcgacagagtgaccatcacatgcaaggcctctcaggacgtgggaatcgccgtggcttggtatcagcagaaacctggcaaggtgcccaagctgctgatctactgggcctccaccagacataccggcgtgcccgatagattctccggctctggctctggcaccgactttaccctgaccatctctagcctccagcctgaggatgtggccacctactactgccagcagtacagcagctacccctacacctttggacagggcaccaaggtggaaatcaagtccggaggtggcggctccgacaagacccatacttgtcctccatgtcctgctccagaggctgctggtggcccttccgttttcctgtttccacctaagcctaaggacaccctgatgatctctcggacccctgaagtgacctgcgtggtggtcgatgtgtctcacgaggacccagaagtgaagttcaattggtacgtggacggcgtggaagtgcacaatgccaagaccaagcctagagaggaacagtacgcctccacctacagagtggtgtctgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccttgcctcctagccgggacgagctgaccaagaatcaggtgtccctgtcctgtgccgtgaagggcttctacccttccgatatcgctgtggaatgggagagcaacggccagccagagaacaactacaagaccacacctcctgtgctggacagcgacggctcattcttcctggtgtccaagctgacagtggacaagtccagatggcagcagggcaacgtgttctcctgctccgtgatgcacgaggccctgcacaatcactacacacagaagtccctgagcctgtctccaggcaagtgatga

SEQ ID NO: 27 - DNA, 1125 bp, artificial sequence
  VHUCHT1 human IgG1FC hole coding sequence
atggaaacagataccctgctggtgttcgtgctgctcgtgtgggtgccagctggaaatggcgaagtgcagctggttgaatcaggtggtggtctggttcagccaggtggtagcctgcgtctgagctgtgcagcaagtggttattcttttaccggttataccatgaattgggtgcgccaagctccgggtaaaggcctggaatgggttgcactgattaatccgtataaaggtgtgagcacctataatcagaaattcaaagatcgctttaccatcagcgtggataaaagcaaaaataccgcctacctgcaaatgaatagtctgcgtgcagaggatacagcagtgtattattgtgcacgtagcggttattatggtgatagcgattggtatttcgacgtgtggggacaaggtacactggtgaccgtgtccggaggtggcggctccgacaagacccatacttgtcctccatgtcctgctccagaggctgctggtggcccttccgttttcctgtttccacctaagcctaaggacaccctgatgatctctcggacccctgaagtgacctgcgtggtggtcgatgtgtctcacgaggacccagaagtgaagttcaattggtacgtggacggcgtggaagtgcacaatgccaagaccaagcctagagaggaacagtacgcctccacctacagagtggtgtctgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccttgcctcctagccgggacgagctgaccaagaatcaggtgtccctgtcctgtgccgtgaagggcttctacccttccgatatcgctgtggaatgggagagcaacggccagccagagaacaactacaagaccacacctcctgtgctggacagcgacggctcattcttcctggtgtccaagctgacagtggacaagtccagatggcagcagggcaacgtgttctcctgctccgtgatgcacgaggccctgcacaatcactacacacagaagtccctgagcctgtctccaggcaagtgatga

SEQ ID NO: 28 - DNA, 1530 bp, artificial sequence
  scFv antiCD38 human IgG1FC knob coding sequence
atggaaacagataccctgctggtgttcgtgctgctcgtgtgggtgccagctggaaatggacaggtgcagctggtggaatctggcggaggattggttcagcctggcggctctctgagactgtcttgtgccgcttccggcttcaccttctccagctacggaatgcactgggtccgacaggcccctggcaaaggattggagtgggtgtccaacatctactccgacggctccaacaccttctacgccgactctgtgaagggcagattcaccatcagccgggacaactccaagaacaccctgtacctccagatgaactccctgagagccgaggacaccgccgtgtactactgcgccagaaacatgtaccggtggcctttccactacttcttcgactactggggccagggcaccctggtcacagtttctagcggcggaggtggaagcggaggcggaggtagtggtggtggcggatctgatatcgagctgacccagcctccttccgtgtctgttgctcctggccagaccgccagaatctcttgctctggcgacaacatcggcaacaaatacgtgtcctggtatcagcagaagcccggccaggctcctgtggtggttatctacggcgataacaaccggcctagcggcatccctgaacggttctccggctctaactccggcaataccgccacactgaccatctctggcacacaggctgaggacgaggccgactactactgctcctcctacgactcctcttacttcgtgttcggcggaggcaccaagctgacagttttgggacaatccggaggcggcggaagcgacaagacccatacttgtcctccatgtcctgctccagaggctgctggtggcccttctgtgttcctgtttcctccaaagcctaaggacaccctgatgatctctcggacccctgaagtgacctgcgtggtggtcgatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtttccaacaaggccctgcctgctcctatcgaaaagaccatctccaaggccaagggccagcctagggaaccccaggtttacaccttgcctccatgccgggatgagctgaccaagaaccaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgactgtggacaagtccagatggcagcagggcaacgtgttctcctgctccgtgatgcacgaggccctgcacaatcactacacacagaagtccctgtctctgagccccggaaaggtcgacggctctcaccaccaccatcaccactgatga

SEQ ID NO: 29 - protein, 107 aa, Artificial Sequence
  CH3 hole chain
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 117 aa, Artificial sequence
  CH3 Knob chain
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKVDGSHHHHHH

SEQ ID NO: 31 - protein, 370 aa, Artificial Sequence
  VL diL2K IgG1 Fc Knob
METDTLLVFVLLVWVPAGNGDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKVDGSHHHHHH

SEQ ID NO: 32 - DNA, 1116 bp, Artificial sequence
  coding sequence VL diL2K IgG1 Fc knob
atggagacagataccctgctggtgttcgtgctgctcgtgtgggtgccagctggaaacggcgatatcgtgctgacccagtctcctgccacactgtcactgtctccaggcgagagagctaccctgtcttgcagagcctctcagtccgtgtcctacatgaactggtatcagcagaagcccggcaaggcccctaagcggtggatctacgatacctccaaggtggcctctggcgtgccagccagattttctggctctggatctggcaccgactactccctgaccatcaactccctggaagccgaggatgccgccacctactattgtcagcagtggtctagcaaccctctgacctttggcggaggcaccaaggtggaaatcaaaggctccggaggcggcggaagcgacaagacccatacttgtcctccatgtcctgctccagaggctgctggtggcccttctgtgttcctgtttcctccaaagcctaaggacaccctgatgatctctcggacccctgaagtgacctgcgtggtggtcgatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtttccaacaaggccctgcctgctcctatcgaaaagaccatctccaaggccaagggccagcctagggaaccccaggtttacaccttgcctccatgccgggatgagctgaccaagaaccaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgactgtggacaagtccagatggcagcagggcaacgtgttctcctgctccgtgatgcacgaggccctgcacaatcactacacacagaagtccctgtctctgagccccggaaaggtcgacggctctcaccaccaccatcaccactgatga

SEQ ID NO: 33 - protein, 330 aa, Homo sapiens [sequence omitted]

SEQ ID NO: 34 - protein, 14 aa, Artificial Sequence
  Fc Knob peptide AA 360-373
KNQVSLTCLVKGFY

SEQ ID NO: 35 - protein, 14 aa, Artificial sequence
  Fc knob peptide AA 360-373 T366W mutation
KNQVSLWCLVKGFY

SEQ ID NO: 36 - protein, 14 aa, Artificial sequence
  Fc knob peptide AA 348-361
VYTLPPSRDELTKN

SEQ ID NO: 37 - protein, 14 aa, Artificial sequence
  Fc knob peptide AA 348-361 S354C mutation
VYTLPPCRDELTKN

SEQ ID NO: 38 - protein, 14 aa, artificial sequence
  Fc hole peptide AA360-373
KNQVSLTCLVKGFY

SEQ ID NO: 39 - protein, 14 aa, artificial sequence
  Fc hole peptide AA 360-373 T366S and L368A mutation
KNQVSLSCAVKGFY

SEQ ID NO: 40 - protein, 14 aa, artificial sequence
  Fc hole peptide AA 401-414
DGSFFLYSKLTVDK

SEQ ID NO: 41 - protein, 14 aa, artificial sequence
  Fc hole peptide AA 401-414 Y407V mutation
DGSFFLVSKLTVDK

SEQ ID NO: 42 - protein, 14 aa, artificial sequence
  Fc hole AA 434-356
PREPQVYTLPPSRD

SEQ ID NO: 43 - protein, 14 aa, artificial sequence
  Fc hole peptide AA 343-356 Y349C mutation
PREPQVCTLPPSRD

SEQ ID NO: 44 - protein, 14 aa, artificial sequence
  CH2 region AA 228-241
PCPAPELLGGPSVF

SEQ ID NO: 45 - protein, 14 aa, artificial sequence
  CH2 region AA 228-241 effector silencing L234A and L235A (LALA) mutation
PCPAPEAAGGPSVF

SEQ ID NO: 46 - protein, 14 aa, artificial sequence
  CH2 region AA 291-304
PREEQYNSTYRVVS

SEQ ID NO: 47 - protein, 14 aa, artificial sequence
  CH2 region AA 291-304 effector silencing N297A mutation
PREEQYASTYRVVS

SEQ ID NO: 48 - protein, 14 aa, artificial sequence
  CH2 region AA 323-336
VSNKALPAPIEKTI

SEQ ID NO: 49 - protein, 14 aa, artificial sequence
  CH2 region AA 323-336 effector silencing P329A mutation
VSNKALAAPIEKTI

SEQ ID NO: 50 - DNA, 1275 bp, Artificial Sequence
  Darpin antiROR1 G3w human IgG1 Fc knob
atggaaaccgacacactgctggtgttcgtgctgctcgtgtgggtgccagctggaaacggcgaattcgatctgggtaaaaaactgctggaagcagcacgtgcaggtcaggatgatgaagttcgtattctgatggcaaatggtgcagatgttaatgccaacgattatattggtcgtacaccgctgcatctggcagcagatagcggtcatctggaaattgttgaagttctgctgaaacatggtgccgatgtgaacgcaaccgatgcatggggtattaccccgttacacctggcagcatgggcaggccatttagaaatcgtggaagttttactgaaatatggcgacgatgtaaatgcagcagattcagatggtaatacgcctctgcatttagcagcatattcaggccacctggaaatagtagaggtgctgttaaaatacggtgccgacgttaatgcacaggataaatttggtaaaaccgcctttgatattagcatcgataatggcaatgaagatctggccgaaattctgcaaaaactgaatggatccggaggcggcggaagcgacaagacccatacttgtcctccatgtcctgctccagaggctgctggtggcccttctgtgttcctgtttcctccaaagcctaaggacaccctgatgatctctcggacccctgaagtgacctgcgtggtggtcgatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtttccaacaaggccctgcctgctcctatcgaaaagaccatctccaaggccaagggccagcctagggaaccccaggtttacaccttgcctccatgccgggatgagctgaccaagaaccaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgactgtggacaagtccagatggcagcagggcaacgtgttctcctgctccgtgatgcacgaggccctgcacaatcactacacacagaagtccctgtctctgagccccggaaaggtcgacggctctcaccaccaccatcaccactgatga

SEQ ID NO: 51 - protein, 422 aa, Artificial Sequence
  Darpin antiROR1 G3w human IgG1 Fc knob
METDTLLVFVLLVWVPAGNGEFDLGKKLLEAARAGQDDEVRILMANGADVNANDYIGRTPLHLAADSGHLEIVEVLLKHGADVNATDAWGITPLHLAAWAGHLEIVEVLLKYGDDVNAADSDGNTPLHLAAYSGHLEIVEVLLKYGADVNAQDKFGKTAFDISIDNGNEDLAEILQKLNGSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKVDGSHHHHH